(19) United States
(12) Patent Application Publication
(10) Pub. No.: US 2025/0011384 A1
(43) Pub. Date: Jan. 9, 2025

(54) CYTOKINE-IMMUNORECEPTOR FUSION PROTEINS AND USES THEREOF

(71) Applicant: Senti Biosciences, Inc., South San Francisco, CA (US)

(72) Inventors: Aldo Omar Sotelo, San Francisco, CA (US); Alba Gonzalez-Junca, Castro Valley, CA (US); Nicholas Frankel, San Francisco, CA (US)

(21) Appl. No.: 18/777,176

(22) Filed: Jul. 18, 2024

Related U.S. Application Data
(63) Continuation of application No. PCT/US2023/060859, filed on Jan. 18, 2023.
(60) Provisional application No. 63/300,456, filed on Jan. 18, 2022.

(51) Int. Cl. C07K 14/54; A61K 35/17; A61K 39/00; C07K 14/715; C12N 5/0783

(52) U.S. Cl. CPC C07K 14/5443; A61K 35/17; A61K 39/4613; A61K 39/4635; C07K 14/7155; C12N 5/0646; C07K 2319/02; C07K 2319/03

(57) ABSTRACT

Provided herein are cytokine-immunoreceptor fusion proteins. Also provided herein are cells, polynucleotides, vectors, compositions, and methods directed to cytokine-immunoreceptor fusion proteins.

### CROSS REFERENCE TO RELATED APPLICATIONS

This application is a continuation of International (PCT) Application No. PCT/US2023/060859 filed Jan. 18, 2023, which claims the benefit of and priority to U.S. Provisional Application No. 63/300,456, filed Jan. 18, 2022, the disclosures of each of which are hereby incorporated by reference in their entireties for all purposes.

### SEQUENCE LISTING

The instant application contains a Sequence Listing XML which has been submitted electronically and is hereby incorporated by reference in its entirety. Said XML copy, created on May 4, 2023, is named STB-036WO, and is 144,223 bytes in size.

### BACKGROUND

Chimeric antigen receptor (CAR) based adoptive cell therapies used to redirect the specificity and function of immunoresponsive cells, such as T cells, have shown efficacy in patients with lymphoid malignancies (Pule et al., Nat. Med. (14): 1264-1270 (2008); Maude et al., N Engl J Med. (371): 1507-17 (2014); Brentjens et al., Sci Transl Med. (5): 177ra38 (2013)). CAR-T cells have been shown to induce complete remission in patients with CD19-expressing malignancies for whom chemotherapies have led to drug resistance and tumor progression. The success of CD19 CAR therapy provides optimism for treating other malignancies, such as solid tumors.

IL-15 (also known as Interleukin 15 or MGC9721) is a cytokine that has been described as a T cell growth factor. IL-15 cytokines belong to the four α-helix bundle family, and the membrane receptor comprises two subunits (the IL-15Rβ and γ chains) responsible for signal transduction. IL-15 has demonstrated critical functions in the activation and survival of NK cells and CD8+ cytotoxic T cells (CD8+CD44hi memory T cells) to mediate a prolonged immune response against pathogens and tumors. IL-15 fusion proteins of IL-15 and IL15Rα (IL-15/IL-15Ra) have been expressed on the membrane of engineered CAR cells, such as NK-CARs to enhance their survival and anti-tumor immunity (see U.S. Pat. Nos. 9,629,877 and 10,358,477). However, this method has potential to exhaust NK-CAR cells through tonic cytokine signaling induced by the intracellular domain of IL-15Rα.

Accordingly, there remains a need for improved cytokine fusion proteins, e.g., IL-15 fusion proteins, for use in the treatment of cancer.

### SUMMARY

In one aspect, provided herein is a fusion protein comprising:
(a) an extracellular domain (ECD) comprising a cytokine;
(b) a hinge, and
(c) a transmembrane domain.

In some embodiments, the fusion protein further comprises an intracellular domain (ICD) comprising an intracellular signaling domain derived from an activating immune cell receptor.

In some embodiments, the ECD comprises a cytokine selected from the group consisting of: IL-15, IL1-beta, IL-2, IL-4, IL-6, IL-7, IL-10, IL-12, IL-17A, IL-18, IL-21, IL-22, Type I interferons, Interferon-gamma, TNF-alpha, CCL21a, CXCL10, CXCL11, CXCL13, CCL19, CXCL9, XCL1, mutants thereof, fragments thereof, and fusion proteins thereof.

In some embodiments, the cytokine is a chemokine.

In some embodiments, the ECD comprises an IL-15. In some embodiments, the ECD comprises an IL-15 and an IL-15 receptor. In some embodiments, the ECD comprises an IL-15 and an IL-15Rα. In some embodiments, the ECD comprises an IL-15 and a sushi domain of IL-15Rα.

In some embodiments, the fusion protein further comprises a signal sequence. In some embodiments, the signal sequence comprises a CD8 signal sequence or an IgE signal sequence. In some embodiments, the signal sequence comprises the amino acid sequence of SEQ ID NO:70.

In some embodiments, the IL-15 and the sushi domain of IL-15Rα are connected via a linker. In some embodiments, the linker comprises a glycine-serine linker. In some embodiments, the linker comprises the amino acid sequence of SEQ ID NO:15.

In some embodiments, the IL-15 comprises the amino acid sequence of SEQ ID NO: 1. In some embodiments, the sushi domain of IL-15Rα comprises the amino acid sequence of SEQ ID NO:2.

In some embodiments, the ECD comprises the amino acid sequence of SEQ ID NO: 85. In some embodiments, the ECD does not comprise a non-sushi domain of IL-15Rα.

In some embodiments, the ECD further comprises the amino acid sequence of SEQ ID NO: 104.

In some embodiments, the transmembrane domain comprises a transmembrane domain derived from an activating immune cell receptor. In some embodiments, the transmembrane domain comprises a transmembrane domain from a membrane protein selected from the group consisting of: CD25, CD7, CD3zeta, CD4, 4-1BB, ICOS, CTLA-4, LAX, LAT, PD-1, LAG-3, TIM3, LIR-1 KIR3DS1, KIR3DL1, NKG2D, NKG2A, TIGIT, BTLA, IL-15Rα, CD28, OX40, CD8a, NKp46, and 2B4.

In some embodiments, the transmembrane domain comprises an IL-15Rα transmembrane domain. In some embodiments, the IL-15Rα transmembrane domain comprises the amino acid sequence of SEQ ID NO:5.

In some embodiments, the transmembrane domain comprises a CD28 transmembrane domain. In some embodiments, the CD28 transmembrane domain comprises the amino acid sequence of SEQ ID NO:7.

In some embodiments, the transmembrane domain comprises an OX40 transmembrane domain. In some embodiments, the OX40 transmembrane domain comprises the amino acid sequence of SEQ ID NO:9.

In some embodiments, the transmembrane domain comprises a CD8 transmembrane domain. In some embodiments, the CD8 transmembrane domain comprises the amino acid sequence of SEQ ID NO:36.

In some embodiments, the transmembrane domain comprises an NKp46 transmembrane domain. In some embodiments, the NKp46 transmembrane domain comprises the amino acid sequence of SEQ ID NO: 12.

In some embodiments, the transmembrane domain comprises a 2B4 transmembrane domain. In some embodiments, the 2B4 transmembrane domain comprises the amino acid sequence of SEQ ID NO:14.

In some embodiments, the transmembrane domain and the ICD are derived from the same activating immune cell receptor.

In some embodiments, the transmembrane domain and the ICD are derived from different activating immune cell receptors.

In some embodiments, the hinge is derived from an activating immune cell receptor. In some embodiments, the hinge is derived from a protein selected from the group consisting of: IgG4, IgG2, IgD, KIR2DS2, LNGFR, PDGFR, CD28, CD8a, and MAG.

In some embodiments, the hinge is derived from CD28. In some embodiments, the hinge comprises the amino acid sequence of SEQ ID NO:40.

In some embodiments, the hinge is derived from CD8a. In some embodiments, the hinge comprises the amino acid sequence of SEQ ID NO:49.

In some embodiments, the hinge is derived from MAG. In some embodiments, the hinge comprises the amino acid sequence of SEQ ID NO:3.

In some embodiments, the hinge and the ICD are derived from the same activating immune cell receptor.

In some embodiments, the hinge and the ICD are derived from different activating immune cell receptors.

In some embodiments, the ICD does not comprise an IL-15Rα intracellular domain.

In some embodiments, the ICD comprises a signaling domain derived from a membrane protein selected from the group consisting of: CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In some embodiments, the ICD comprises an intracellular signaling domain derived from CD28. In some embodiments, the ICD comprises the amino acid sequence of SEQ ID NO: 18.

In some embodiments, the ICD comprises an intracellular signaling domain derived from OX40. In some embodiments, the ICD comprises the amino acid sequence of SEQ ID NO: 64.

In some embodiments, the ICD comprises an intracellular signaling domain derived from 4-1BB. In some embodiments, the ICD comprises the amino acid sequence of SEQ ID NO: 66.

In some embodiments, the ICD comprises an intracellular signaling domain derived from NKp46. In some embodiments, the ICD comprises the amino acid sequence of SEQ ID NO: 67.

In some embodiments, the ICD comprises an intracellular signaling domain derived from 2B4. In some embodiments, the ICD comprises the amino acid sequence of SEQ ID NO: 69.

In some embodiments, the fusion protein comprises, from N-terminus to C-terminus, an IgE signal sequence, the ECD, the hinge, the transmembrane domain, and an ICD.

In another aspect, provided herein is a nucleotide construct encoding the fusion protein of any one of the above embodiments.

In another aspect, provided herein is a vector comprising the above aspect.

In another aspect, provided herein is an immunoresponsive cell comprising the fusion protein, the nucleotide construct, or the vector of any of the above embodiments or aspects.

In some embodiments, the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the immunoresponsive cell is a T cell. In some embodiments, the immunoresponsive cell is an NK cell.

In some embodiments, the immunoresponsive cell further comprises an activating immune receptor. In some embodiments, the immunoresponsive cell further comprises a chimeric antigen receptor (CAR).

In some embodiments, the CAR comprises:
an antigen-binding domain specific for a target antigen,
a transmembrane domain, and
an intracellular signaling domain (ICD).

In some embodiments, the target antigen is a tumor-associated antigen, a bacterial antigen, a viral antigen, or a self-antigen. In some embodiments, the CAR comprises a transmembrane domain derived from an activating immune cell receptor. In some embodiments, the CAR comprises an ICD of an activating immune cell receptor. In some embodiments, the ICD of the CAR is different from the ICD of the fusion protein. In some embodiments, the ICD of the CAR is the same as the ICD of the fusion protein. In some embodiments, the CAR comprises a CD3zeta ICD. In some embodiments, the CAR does not comprise a co-stimulatory domain. In some embodiments, a costimulatory signal is provided from the fusion protein.

In some embodiments, the immunoresponsive cell is allogeneic.

In some embodiments, the immunoresponsive cell is autologous.

In another aspect, provided herein is a pharmaceutical composition comprising the fusion protein, the vector, or the immunoresponsive cell of any one of the above aspects or embodiments, and a pharmaceutically acceptable carrier, pharmaceutically acceptable excipient, or a combination thereof.

In another aspect, provided herein is a method of treating a subject in need thereof, the method comprising administering to a subject a therapeutically effective dose of the fusion protein, the immunoresponsive cell, or the pharmaceutical composition of any one of the above aspects or embodiments.

In another aspect, provided herein is a method of stimulating an immune response in a subject, the method comprising administering to a subject a therapeutically effective dose of the fusion protein, the immunoresponsive cell, or the pharmaceutical composition of any one of the above aspects or embodiments.

In another aspect, provided herein is a method of reducing tumor volume in a subject, the method comprising administering to a subject having a tumor a composition comprising the fusion protein, the immunoresponsive cell, or the pharmaceutical composition of any one of the above aspects or embodiments.

In another aspect, provided herein is a method of providing an anti-tumor immunity in a subject, the method comprising administering to a subject in need thereof a therapeutically effective dose of the fusion protein, the immunoresponsive cell, or the pharmaceutical composition of any one of the above aspects or embodiments.

In another aspect, provided herein is a method of treating a subject having cancer, the method comprising administering a therapeutically effective dose of the fusion protein, the immunoresponsive cell, or the pharmaceutical composition of any one of the above aspects or embodiments.

In another aspect, provided herein is a kit for treating and/or preventing a tumor, comprising the fusion protein of any one of the above aspects or embodiments. In some embodiments, the kit further comprises written instructions for using the fusion protein for treating and/or preventing a tumor in a subject.

In another aspect, provided herein is a kit for treating and/or preventing a tumor, comprising the immunoresponsive cell of any one of the above aspects or embodiments. In some embodiments, the kit further comprises written instructions for using the immunoresponsive cell for treating and/or preventing a tumor in a subject.

In another aspect, provided herein is a kit for treating and/or preventing a tumor, comprising the pharmaceutical composition of the above aspect. In some embodiments, the kit further comprises written instructions for using the pharmaceutical composition for treating and/or preventing a tumor in a subject.

### BRIEF DESCRIPTION OF THE DRAWINGS

FIGS. 1A and 1B depict a schematic detailing the structure of the fusion proteins described herein. SB05497, SB05498, SB05499, SB05500, SB05501, and SB05502 are shown in FIG. 1A; SB05497, SB05501, SB05917, SB05918, and SB05919 are shown in FIG. 1B.

FIG. 2A shows flow cytometry plots of NK-cells transduced with IL-15/IL15Rα fusion proteins for IL-15Rα expression. FIG. 2B is a distribution plot of IL-15Rα expression, and FIG. 2C is a histogram quantifying the percentage of fusion protein-expressing cells. Mean fluorescent intensity (MFI) is shown in FIG. 2D.

FIG. 3A shows flow cytometry plots of NK-cells transduced with IL-15/IL15Rα fusion proteins, starved of cytokines, and stained for pSTAT5. FIG. 3B is a histogram quantifying the percentage of pSTAT5-expressing cells.

FIG. 4 shows flow cytometry plots of NK-cells transduced with IL-15/IL15Rα fusion proteins, starved of cytokines, and stained for pSTAT5.

FIG. 5A shows percent viable cells by Celleca MX for fusion-protein expressing cells starved of cytokines for 5 days; FIG. 5B shows the fold increase in viable cells. FIG. 5C shows percent viable cells by Celleca MX for fusion-protein expressing cells starved of cytokines for 6 days; FIG. 5D shows the fold increase in viable cells.

FIG. 6A shows fold increase of viable NK cells expressing fusion proteins following culture with IL-2. Normalized results of a serial killing assay against target cells are shown in FIG. 6B (1 round of killing) and FIG. 6C (2 rounds of killing). Relative target cell abundance is shown in FIG. 6D (1 round of killing) and FIG. 6E (2 rounds of killing).

FIG. 7A shows fold increase of viable NK cells expressing fusion proteins following culture with IL-2. Relative target cell abundance following a serial killing assay against target cells are shown in FIG. 7B (1 round of killing) and FIG. 7C (2 rounds of killing).

FIG. 8A shows fold increase of viable NK cells expressing fusion proteins following culture with IL-2. Relative target cell abundance following a serial killing assay against target cells are shown in FIG. 8B (1 round of killing) and FIG. 8C (2 rounds of killing).

FIG. 9A shows fold increase of viable NK cells expressing fusion proteins following culture with IL-2. Relative target cell abundance following a serial killing assay against target cells are shown in FIG. 9B (1 round of killing) and FIG. 9C (2 rounds of killing).

FIG. 10A shows fold increase of viable NK cells expressing fusion proteins following culture with IL-2. Relative target cell abundance following a serial killing assay against target cells are shown in FIG. 10B (1 round of killing) and FIG. 10C (2 rounds of killing).

FIGS. 11A-11D show comparative results in a serial killing assay. Normalized killing is shown in FIG. 11A (1 round of killing) and FIG. 11B (2 rounds of killing). Relative target cell abundance is shown in FIG. 11C (1 round of killing) and FIG. 11D (2 rounds of killing).

FIG. 12A is a summary of fold increase of viable NK cells expressing fusion proteins of the present disclosure. FIG. 12B is a summary of relative target cell abundance following 2 rounds of killing by said NK cells.

FIGS. 13A-13B detail characterization of chimeric IL15R fusion proteins. FIG. 13A shows killing of target cells incubated with control or NK cells expressing indicated constructs. FIG. 13B shows second round killing of target cells incubated with control or CAR-NK cells expressing the indicated constructs.

### DETAILED DESCRIPTION

The practice of the present disclosure will employ, unless otherwise indicated, conventional methods of molecular biology, chemistry, biochemistry, virology, and immunology, within the skill of the art. Such techniques are explained fully in the literature. See, e.g., Hepatitis C Viruses: Genomes and Molecular Biology (S. L. Tan ed., Taylor & Francis, 2006); Fundamental Virology, 3^{rd} Edition, vol. I & II (B. N. Fields and D. M. Knipe, eds.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.).

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

As used herein, the term “stimulating a cell-mediated immune response” or “stimulating an immune response” refers to generating a signal that results in an immune response by one or more cell types or cell populations. Immunostimulatory activity may include pro-inflammatory activity. In various embodiments, the immune response occurs after immune cell (e.g., T-cell or NK cell) activation or concomitantly mediated through receptors including, but not limited to, CD28, CD137 (4-1BB), OX40, CD40 and ICOS, and their corresponding ligands, including B7-1, B7-2, OX-40L, and 4-1BBL. Such polypeptides may be present in the tumor microenvironment and can activate immune responses to neoplastic cells. In various embodiments, promoting, stimulating, or otherwise agonizing pro-inflammatory polypeptides and/or their ligands may enhance the immune response of an immunoresponsive cell. Without being bound to a particular theory, receiving multiple stimulatory signals (e.g., co-stimulation) is important to mount a robust and long-term cell-mediated immune response, such as a T cell mediated immune response where T cells can become inhibited and unresponsive to antigen (also referred to as “T cell anergy”) in the absence of co-stimulatory signals. While the effects of the variety of co-stimulatory signals, particularly in combination with one another, can vary and remain only partially understood, co-stimulation generally results in increasing gene expression in order to generate long-lived, proliferative, and apoptotic resistant cells, such as T cells or NK cells, that robustly respond to antigen, for example in meditating complete and/or sustained eradication of targets cells expressing a cognate antigen.

As used herein, “an activating immune cell receptor” is a receptor that produces an immune cell activation signal. Exemplary activating immune cell receptors include, for example and without limitation, CD25, CD7, CD3zeta, CD4, 4-1BB, ICOS, CTLA-4, LAX, LAT, PD-1, LAG-3, TIM3, LIR-1 KIR3DS1, KIR3DL1, NKG2D, NKG2A, TIGIT, BTLA, IL-15Rα, CD28, OX40, CD8, NKp46, and 2B4. As used herein, an activating immune cell receptor when used in an intracellular domain (ICD) of a fusion protein of the present disclosure is derived from a different protein than the protein used in the extracellular domain (ECD) of the fusion protein.

As used herein, a “fusion protein” is a polypeptide that comprises two or more regions derived from different or heterologous proteins or peptides.

As used herein, the term “chimeric antigen receptor” or alternatively a “CAR” refers to a recombinant polypeptide construct comprising at least an extracellular antigen-binding domain, a transmembrane domain and a cytoplasmic signaling domain (also referred to herein as “an intracellular signaling domain”) comprising a functional signaling domain.

As used herein, the term “activating CAR” or “aCAR” refers to CAR constructs/architectures capable of inducing signal transduction or changes in protein expression in the activating CAR-expressing cell that initiate, activate, stimulate, or increase an immune response upon binding to a cognate aCAR ligand.

As used herein, the term “inhibitory CAR” or “iCAR” refers to CAR constructs/architectures capable of inducing signal transduction or changes in protein expression in the inhibitory CAR-expressing cell that prevent, attenuate, inhibit, reduce, decrease, inhibit, or suppress an immune response upon binding to a cognate iCAR ligand, such as reduced activation of immunoresponsive cells receiving or having received one or more stimulatory signals, including co-stimulatory signals.

As used herein, the term “native cognate receptor” or “native receptor” as applied to a cytokine refers to a native receptor that is capable of binding (e.g., known to bind) to the cytokine. For example, native cognate receptors to IL15 can include, but are not limited to, IL15 receptor, IL15 receptor alpha (IL15Rα), IL15 receptor beta, and/or IL15 receptor gamma.

As used herein, “derived from” indicates a relationship between a first molecule and a second molecule (e.g., between polynucleotides, polypeptides, etc.). The term “derived from” may refer to a functional and/or structural similarity between a first molecule and a second molecule. In many cases, a first molecule is considered to be derived from a second molecule (e.g., a reference molecule) when said first molecule has been modified relative to said second molecule, where said first molecule does not necessarily comprise the same sequence as said second molecule (e.g., nucleic acid sequence, amino acid sequence, etc.), but has at least 1%, 5%, 10%, 20%, 30%, 40%, 50%, 60%, 70%, 80%, 90%, 95%, 96%, 97%, 98%, 99% or more of an activity of said second molecule as determined in a suitable assay. In some cases, the first molecule has at least 50%, at least 60%, at least 70%, at least 80%, at least 90%, at least 95%, at least 96%, at least 97%, at least 98%, at least 99% or more of an activity of said second molecule as determined in a suitable assay. For example, a polypeptide may be considered to be derived from a reference polypeptide when said polypeptide contains one or more amino acid modifications relative to said reference polypeptide and retains certain functions, such as certain intermolecular or intramolecular interactions (e.g., binding to a protein, e.g., a particular receptor, signaling activity), though such interactions could be stronger, equivalent, or weaker than that of the reference polypeptide. Additionally, a first molecule may be considered to be derived from a second molecule (e.g., reference molecule) when said first molecule comprises at least 10%, 20%, 30%, 40%, 50%, 60%, 70%, 80%, 90%, 91%, 92%, 93%, 94%, 95%, 96%, 97%, 98%, 99% or more sequence identity (e.g., nucleic acid sequence identity, amino acid sequence identity, etc.) to at least 10%, 20%, 30%, 40%, 50%, 60%, 70%, 80%, 90%, 91%, 92%, 93%, 94%, 95%, 96%, 97%, 98%, 99%, or more of the sequence (e.g., nucleic acid sequence, amino acid sequence, etc.) of the second molecule (but not necessarily to a contiguous portion of a sequence). In some cases, the first molecule comprises at least 90%, at least 95%, at least 96%, at least 97%, at least 98%, at least 99% or more sequence identity to at least 50%, 60%, 70%, 80%, 90%, 91%, 92%, 93%, 94%, 95%, 96%, 97%, 98%, 99%, or more of the sequence (e.g., nucleic acid sequence, amino acid sequence, etc.) of the second molecule (but not necessarily to a contiguous portion of a sequence). As another non-limiting example, a polynucleotide may be considered to be derived from a reference polynucleotide when said polynucleotide contains one or more nucleotide modifications relative to the reference polynucleotide, but still encodes a protein or protein fragment that functions the same as or similar to the protein encoded by the reference polynucleotide, or has the same or similar function (e.g., as a regulatory element, e.g., promoter or enhancer) as the reference polynucleotide. In addition, molecules discussed herein may be a “free-standing” functional unit or system and/or part of a larger system, such as, for example, a vector (e.g., a gene cassette). In some embodiments, the term “derived from” when applied to the intracellular signaling domain of an activating immune cell receptor refers to an amino acid sequence comprising the intracellular signaling domain amino acid sequence as known in the art. Similarly, in some embodiments, the present disclosure provides for transmembrane domains derived from an activating immune cell receptor. In some embodiments, the present disclosure provides for a hinge derived from an activating immune cell receptor. The term “derived from” as used herein will be readily understood by a skilled artisan, e.g, based on the context of its use.

As used herein, the term “intracellular signaling domain” refers to the functional portion of a protein which acts by transmitting information within the cell to regulate cellular activity via defined signaling pathways by generating second messengers or functioning as effectors by responding to such messengers.

As used herein, the term “extracellular antigen-binding domain” or “antigen-binding domain” (ABD) refers to a polypeptide sequence or polypeptide complex that specifically recognizes or binds to a given antigen or epitope, such as the polypeptide sequence or polypeptide complex portion of the chimeric proteins described herein that provide the target antigen specific binding. An ABD (e.g., a receptor, a ligand-binding domain of a receptor, a receptor ligand, a receptor-binding domain, an antibody, an antigen-binding fragment, and/or a chimeric protein including the same) is said to “recognize” the epitope (or more generally, the antigen) to which the ABD specifically binds, and the epitope is said to be the “recognition specificity” or “binding specificity” of the ABD. The ABD is said to bind to its specific antigen or epitope with a particular affinity. As described herein, “affinity” refers to the strength of interaction of non-covalent intermolecular forces between one molecule and another. The affinity, i.e., the strength of the interaction, can be expressed as a dissociation equilibrium constant (KD), wherein a lower KD value refers to a stronger interaction between molecules. KD values of antibody constructs are measured by methods well known in the art including, but not limited to, bio-layer interferometry (e.g., Octet/FORTEBIO®), surface plasmon resonance (SPR) technology (e.g., Biacore®), and cell binding assays (e.g., Flow-cytometry). Specific binding, as assessed by affinity, can refer to a binding molecule with an affinity between an ABD and its cognate antigen or epitope in which the KD value is below 10⁻⁶M, 10⁻⁷M, 10⁻⁸M, 10⁻⁹M, or 10⁻¹⁰M. Specific binding can also include recognition and binding of a biological molecule of interest (e.g., a polypeptide) while not specifically recognizing and binding other molecules in a sample, for example, a biological sample, which naturally includes a polypeptide of the present disclosure. In certain embodiments, specific binding refers to binding between an ABD, ligand, antibody, or antigen-binding fragment to a receptor, a ligand, an epitope, an antigen or antigenic determinant in such a manner that binding can be displaced or competed with a second preparation of identical or similar receptor, ligand, epitope, antigen, or antigenic determinant.

An ABD can be an antibody. The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

An ABD can be an antigen-binding fragment of an antibody. As used herein, the term “antigen-binding fragment” refers to at least one portion of an intact antibody, or recombinant variants thereof, that is sufficient to confer recognition and specific binding of the antigen-binding fragment to a target, such as an antigen or epitope. Examples of antigen-binding fragments include, but are not limited to, Fab, Fab′, F(ab′)₂, Fv, scFv, linear antibodies, single domain antibodies such as sdAb (either VL or VH), camelid V_{H}H domains, and multi-specific antibodies formed from antigen-binding fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1 136, 2005). Antigen binding fragments can also be grafted into scaffolds based on polypeptides such as a fibronectin type III (Fn3) (see U.S. Pat. No. 6,703,199, which describes fibronectin polypeptide minibodies).

An ABD can be a ligand or a receptor-binding domain thereof, e.g., a polypeptide which binds to and/or activates a target receptor.

An ABD can be a receptor or a ligand-binding domain thereof.

The number of ABDs in a binding molecule, such as the chimeric proteins described herein, defines the “valency” of the binding molecule. A binding molecule having a single ABD is “monovalent”. A binding molecule having a plurality of ABDs is said to be “multivalent”. A multivalent binding molecule having two ABDs is “bivalent.” A multivalent binding molecule having three ABDs is “trivalent.” A multivalent binding molecule having four ABDs is “tetravalent.” In various multivalent embodiments, all of the plurality of ABDs have the same recognition specificity and can be referred to as a “monospecific multivalent” binding molecule. In other multivalent embodiments, at least two of the plurality of ABDs have different recognition specificities. Such binding molecules are multivalent and “multispecific.” In multivalent embodiments in which the ABDs collectively have two recognition specificities, the binding molecule is “bispecific.” In multivalent embodiments in which the ABDs collectively have three recognition specificities, the binding molecule is “trispecific.” In multivalent embodiments in which the ABDs collectively have a plurality of recognition specificities for different epitopes present on the same antigen, the binding molecule is “multiparatopic.” Multivalent embodiments in which the ABDs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g., a multivalent binding molecule for a specific target, wherein the avidity is the cumulative strength of interaction provided by the affinities of multiple ABDs. Avidity can be measured by the same methods as those used to determine affinity, as described above. In certain embodiments, the avidity of a binding molecule for a specific target is such that the interaction is a specific binding interaction, wherein the avidity between two molecules has a KD value below 10⁻⁶M, 10⁻⁷M, 10⁻⁸M, 10⁻⁹M, or 10⁻¹⁰ M. In certain embodiments, the avidity of a binding molecule for a specific target has a KD value such that the interaction is a specific binding interaction, wherein the one or more affinities of individual ABDs do not have has a KD value that qualifies as specifically binding their respective antigens or epitopes on their own. In certain embodiments, the avidity is the cumulative strength of interaction provided by the affinities of multiple ABDs for separate antigens on a shared specific target or complex, such as separate antigens found on an individual cell. In certain embodiments, the avidity is the cumulative strength of interaction provided by the affinities of multiple ABDs for separate epitopes on a shared individual antigen.

As used herein, the term “single-chain variable fragment” or “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked via a short flexible polypeptide linker, capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, “variable region” refers to a variable sequence that arises from a recombination event, for example, following V, J, and/or D segment recombination in an immunoglobulin gene in a B cell or T cell receptor (TCR) gene in a T cell. In immunoglobulin genes, variable regions are typically defined from the antibody chain from which they are derived, e.g., VH refers to the variable region of an antibody heavy chain and VL refers to the variable region of an antibody light chain. A select VH and select VL can associate together to form an antigen-binding domain that confers antigen specificity and binding affinity.

The term “complementarity determining region” or “CDR,” as used herein, refers to the sequences within antibody variable regions VH and VL which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise ammo acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al, (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and ammo acid residues 24-34 (LCDR1), 50-56 (LCDR2), and 89-97 (LCDR3) in a VL, e.g., a mammalian VL, e.g., a human VL. In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences.

The term “framework region” or “FR,” as used herein, refers to the generally conserved sequences within antibody variable regions VH and VL that act as a scaffold for interspersed CDRs, typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In specific embodiments, the FRs are human sequences. In various embodiments, the FRs are naturally occurring sequences. In various embodiments, the FRs are synthesized sequences including, but not limited, rationally designed sequences.

As used herein, the term “antibody heavy chain” refers to the larger of the two types of polypeptide chains present in antibody molecules in their naturally occurring conformations, and which normally determines the class to which the antibody belongs.

As used herein, the term “antibody light chain” refers to the smaller of the two types of polypeptide chains present in antibody molecules in their naturally occurring conformations. Kappa (κ) and lambda (λ) light chains refer to the two major antibody light chain isotypes.

As used herein, the term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

As used herein, the term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen.

As used herein, the term “anti-tumor effect” or “anti-tumor activity” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place, such as in a prophylactic therapy or treatment.

As used herein, the term “autologous” refers to any material derived from the same subject to whom it is later to be re-introduced into the subject.

As used herein, the term “allogeneic” refers to any material derived from a different animal of the same species as the subject to whom the material is introduced. Two or more subjects are said to be allogeneic to one another when the genes at one or more loci are not identical. In some embodiments, allogeneic material from individuals of the same species may be sufficiently genetically distinct, e.g., at particular genes such as MHC alleles, to interact antigenically. In some embodiments, allogeneic material from individuals of the same species may be sufficiently genetically similar, e.g., at particular genes such as MHC alleles, to not interact antigenically.

Isolated polynucleotide molecules of the present disclosure include any polynucleotide molecule or nucleic acid sequence that encodes a polypeptide of the present disclosure, or fragment thereof. Such polynucleotide molecules need not be 100% homologous or identical with an endogenous nucleic acid sequence, but will typically exhibit substantial identity. Nucleic acid sequences having “substantial identity” or “substantial homology” to an endogenous sequence are typically capable of hybridizing with at least one strand of a double-stranded polynucleotide molecule. As used herein, “hybridize” refers to pairing to form a double-stranded molecule between complementary polynucleotide sequences (e.g., a gene described herein), or portions thereof, under various conditions of stringency. For example, stringent salt concentration may be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency may be accomplished by combining these various conditions as needed.

By “substantially identical” or “substantially homologous” is meant a polypeptide or polynucleotide molecule exhibiting at least about 50% homologous or identical to a reference amino acid sequence (for example, any one of the amino acid sequences described herein) or nucleic acid sequence (for example, any one of the nucleic acid sequences described herein). Preferably, such a sequence is at least about 60%, about 80%, about 85%, about 90%, about 95%, about 99%, or about 100% homologous or identical at the amino acid level or nucleic acid level to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some versions contain an intron(s).

As used herein, the term “ligand” refers to a molecule that binds to a receptor. In particular, the ligand binds a receptor on another cell, allowing for cell-to-cell recognition and/or interaction.

The terms “effective amount” and “therapeutically effective amount” are used interchangeably herein, and refer to an amount of a compound, formulation, material, or composition, as described herein effective to achieve a particular biological result. In some embodiments, an “effective amount” or a “therapeutically effective amount” is an amount sufficient to arrest, ameliorate, or inhibit the continued proliferation, growth, or metastasis of a disease or disorder of interest, e.g., a solid tumor.

As used herein, the term “immunoresponsive cell” refers to a cell that functions in an immune response (e.g., an immune effector response) or a progenitor, or progeny thereof. Examples of immune effector cells include, without limitation, alpha/beta T cells, gamma/delta T cells, B cells, natural killer (NK) cells, natural killer T (NKT) cells, mast cells, and myeloid-derived phagocytes.

As used herein, the term “immune effector response” or “immune effector function” refers to a function or response, e.g., of an immunoresponsive cell, that enhances or promotes an immune attack of a target cell. For example, an immune effector function or response may refer to a property of a T cell or NK cell that promotes killing or the inhibition of growth or proliferation, of a target cell. In the case of a T cell, primary stimulation and co-stimulation are examples of immune effector function or response.

As used herein, the term “flexible polypeptide linker” or “linker” refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Gly-Ser), (SEQ ID NO: 124) or (Gly-Gly-Gly-Ser), (SEQ ID NO: 125), where n is a positive integer equal to or greater than 1. For example, n=1, n=2, n=3, n=4, n=5, n=6, n=7, n=8, n=9, or n=10. In some embodiments, the flexible polypeptide linkers include, but are not limited to, Gly₄Ser [SEQ ID NO: 29] or (Gly₄Ser) 3 [SEQ ID NO: 31]. In other embodiments, the linkers include multiple repeats of (Gly₂Ser), (GlySer) or (Gly₃Ser) [SEQ ID NO: 24]. In some embodiments, the flexible polypeptide linkers include a Whitlow linker (e.g., GSTSGSGKPGSGEGSTKG [SEQ ID NO:34]). In some embodiments, the flexible polypeptide linkers include an (EAAAK) 3 [SEQ ID NO:126] linker. Also included within the scope of the present disclosure are linkers described, for example, in WO2012/138475.

As used herein, the terms “treat”, “treatment” and “treating” refer to the reduction or amelioration of the progression, severity and/or duration of a proliferative disorder (e.g., cancer), or the amelioration of one or more symptoms (preferably, one or more discernible symptoms) of a proliferative disorder resulting from the administration of one or more therapies (e.g., one or more therapeutic agents such as a CAR of the present disclosure). In some embodiments, reduction or amelioration refers to the amelioration of at least one measurable physical parameter of a proliferative disorder, such as growth of a tumor, not necessarily discernible by the patient. In other embodiments, the terms “treat”, “treatment”, and “treating” refer to the inhibition of the progression of a proliferative disorder, either physically by, e.g., stabilization of a discernible symptom, physiologically by, e.g., stabilization of a physical parameter, or both. In some embodiments, reduction or amelioration include reduction or stabilization of tumor size or cancerous cell count.

As used herein, the term “subject” is intended to include living organisms in which an immune response can be elicited (e.g., mammals, human).

Other aspects of the present disclosure are described in the following sections and are within the ambit of the claimed invention.

### Other Interpretational Conventions

Ranges recited herein are understood to be shorthand for all of the values within the range, inclusive of the recited endpoints. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### Cytokine Fusion Proteins

Described herein are cytokine fusion proteins which comprise at least one cytokine domain, a transmembrane domain, and an intracellular domain (ICD) comprising an intracellular signaling domain derived from an activating immune cell receptor. In some embodiments, the cytokine is selected from the group consisting of: IL-15, IL1-beta, IL-2, IL-4, IL-6, IL-7, IL-10, IL-12, IL-17A, IL-18, IL-21, IL-22, Type I interferons, Interferon-gamma, TNF-alpha, mutants thereof, fragments thereof, and fusion proteins thereof. In some embodiments, the cytokine comprises an IL-15. In some embodiments, the cytokine comprises an ILI-beta. In some embodiments, the cytokine comprises an IL-2. In some embodiments, the cytokine comprises an IL-4. In some embodiments, the cytokine comprises an IL-6. In some embodiments, the cytokine comprises an IL-7. In some embodiments, the cytokine comprises an IL-10. In some embodiments, the cytokine comprises an IL-12. In some embodiments, the cytokine comprises an IL-12p70. In some embodiments, the cytokine comprises an IL-17A. In some embodiments, the cytokine comprises an IL-18. In some embodiments, the cytokine comprises an IL-21. In some embodiments, the cytokine comprises an IL-22. In some embodiments, the cytokine comprises a Type 1 interferon. In some embodiments, the cytokine comprises an interferon-gamma. In some embodiments, the cytokine comprises a TNF-alpha. In certain embodiments, the cytokine comprises a fusion protein of a cytokine and one or more additional proteins or domains thereof (e.g., IL-15/IL-15Rα, IL12p70). In certain embodiments, the cytokine comprises a mutant cytokine. In certain embodiments, the cytokine comprises a fragment, e.g., one of more domains, of a cytokine.

In some embodiments, the cytokine fusion protein is a chemokine fusion protein. In some embodiments, the chemokine is selected from the group consisting of: CCL21a, CXCL10, CXCL11, CXCL13, CCL19, CXCL9, and XCL1. In some embodiments, the chemokine comprises a CCL21a. In some embodiments, the chemokine comprises a CXCL10. In some embodiments, the chemokine comprises a CXCL11. In some embodiments, the chemokine comprises a CXCL13. In some embodiments, the chemokine comprises a CCL19. In some embodiments, the chemokine comprises a CCL9. In some embodiments, the chemokine comprises an XCL1. In certain embodiments, the chemokine comprises a fusion protein of a chemokine and one or more additional proteins or domains thereof (e.g., CXCL10-CXCL11 fusion). In certain embodiments, the chemokine comprises a mutant chemokine. In certain embodiments, the chemokine comprises a fragment, e.g., one of more domains, of a chemokine.

### Exemplary IL-15/IL-15Rα-Fusion Proteins

In certain embodiments, the cytokine fusion protein is an IL-15/IL-15Rα-fusion protein which comprises at least one IL-15Rα sushi domain, a transmembrane domain, and an intracellular domain (ICD) comprising an intracellular signaling domain derived from an activating immune cell receptor.

Said fusion proteins may be used, in some embodiments, for stimulating the IL-15Rβ/γ signalling pathway, to induce and/or stimulate the activation, proliferation and/or prevention of apoptosis of IL-15Rβ/γ-positive cells, such as natural killer (NK) and/or T cells. The fusion proteins comprise the sushi domain that is comprised in the extracellular region of an IL-15Rα, e.g., comprising an isolated fragment consisting of IL-15Rα extracellular region, and to sub-fragments thereof that retain the sushi domain.

### IL-15 and Derivatives Thereof

Further to the at least one sushi-containing polypeptide, the fusion proteins described herein comprise at least one IL-15Rβ/γ binding entity. In some embodiments, the IL-15Rβ/γ binding entity may be an IL-15, or an IL-15 fragment, mimetic, or agonist, wherein said IL-15 fragment, mimetic, or agonist has an affinity for binding to IL-15Rβ/γ that is not significantly lower than that of wildtype IL-15. In certain embodiments, the IL-15Rβ/γ binding entity is an IL-15.

In certain embodiments, the IL-15 is, e.g., a human IL-15, or a non-human mammalian IL-15 (e.g., cynomolgus monkey IL-15), or a non-mammalian animal IL-15. Illustrative non-human mammalian IL-15 are monkey IL-15, or a murine IL-15 (e.g., mouse IL-15, NCBI accession number NM_008357; rat IL-15, NCBI accession number NM_013129), a rabbit IL-15 (e.g., NCBI accession number DQ157152), sheep IL-15 (e.g., NCBI accession number NM_001009734), pig IL-15 (e.g., accession number NM_211390), or chicken IL-15 (e.g., accession number NM_204571).

In certain embodiments, the IL-15 is a human IL-15. In certain embodiments, the human IL-15 comprises the amino acid sequence of

(SEQ ID NO: 1)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS.

The terms “agonist” and “mimetic” are herein given their ordinary meaning in the art.

A compound is termed an IL-15 agonist when it induces a biological response that is of a similar or higher level than the one induced by wildtype IL-15. Preferred agonists are those which induce an even higher level of biological response (super-agonist).

An IL-15 agonist typically has an affinity for binding to IL-15Rα and/or to IL-15Rβ/γ that is at least not significantly different from the one of wildtype IL-15, and that is preferably significantly higher than the one of wildtype IL-15. A mimetic of IL-15 refers to a compound capable of mimicking the biological activity of IL-15.

### Sushi Domain of IL-15Rα

Herein, the amino acid sequence of the at least one sushi-domain containing polypeptide comprises the amino acid sequence of the extracellular region of IL-15Rα (the extracellular region of IL-15Rα comprising an IL-15Rα sushi domain), or the amino acid sequence of a fragment of the extracellular region of IL-15Rα, wherein the fragment has retained the sushi domain of the extracellular region of IL-15Rα, wherein the sushi domain is defined as beginning at the first exon 2 encoded cysteine residue (C1), and ending at the fourth exon 2 encoded cysteine residue (C4), residues C1 and C4 being both included in the sushi domain, or

is a variant amino acid sequence that has retained each of the four cysteine residues (C1, C2, C3 and C4) of the sushi domain.

In some embodiments, a fusion protein may comprise a conservative variant sequence of IL-15Rα sushi domain. Such a conservative variant sequence of IL-15Ralpha sushi domain may differ from the wildtype sequence by at least one deletion and/or at least one substitution and/or at least one addition of an amino acid, but the variant retains the biological activity of the wildtype sushi domain.

In some embodiments, an IL-15Rα sushi domain comprises at least 90% sequence identity (e.g., at least 91%, at least 92%, at least 93%, at least 94%, at least 95%, at least 96%, at least 97%, at least 98%, at least 99%, or 100%) to the amino acid sequence of ITCPPPMSVEHADIWVKSYSLYSRERYICNSGFKRKAGTSSLTECVLNKATNVAHWT TPSLKCIR (SEQ ID NO:2). In some embodiments, the IL-15Rα sushi domain comprises the amino acid sequence of SEQ ID NO:2.

In some embodiments, non-human IL-15Rα sushi domains may be used, e.g., those encoded by the nucleic acid sequences available as NCBI accession number NM_008358 (Mus musculus IL-15Rα: nucleic acid sequence of SEQ ID NO:82, amino sequence of SEQ ID NO:81), as accession number XM_521684 (Pan troglodytes IL-15Rα: nucleic acid sequence of SEQ ID NO: 80, amino sequence of SEQ ID NO:79), or as accession number XM_577598 (Rattus norvegicus: IL-15Rα: nucleic acid sequence of SEQ ID NO: 83, amino sequence of SEQ ID NO:84).

Determination of the IL-15Rα “sushi-domain” may also be by analysis of the amino-acid sequence of IL-15Rα with appropriate software such as: Prosite (http://us.expasy.org/prosite/), InterProScan (http://www.ebi.ac.uk/InterProScan/), SMART (http://elm.cu.org/).

### Signal Peptides

A signal peptide (also known in the art as a signal sequence) is a short (e.g., 15-60 amino acids long) peptide chain that directs the post-translational transport of a protein. Some signal peptides are cleaved from the protein by signal peptidase after the proteins are transported. Signal peptides may also be called targeting signals or signal sequences.

A signal sequence used to express a secreted protein can be a homologous signal sequence (derived from the same protein being expressed) or a heterologous signal sequence (derived from a different protein than the one being expressed). In some embodiments, a heterologous signal sequence is from an Ig supergroup member. In some embodiments, the signal sequence is an immunoglobulin chain signal sequence. In some embodiments, the signal sequence is an IgE signal sequence, e.g., having the sequence MDWTWILFLVAAATRVHS (SEQ ID NO:70). In some embodiments, the signal sequence is a CD8 signal sequence e.g., having the sequence MALPVTALLLPLALLLHAARP (SEQ ID NO: 123). In some embodiments, the signal sequence is an Ig Kappa chain V-III region CLL signal peptide, e.g., having the sequence MEAPAQLLFLLLLWLPDTTG (SEQ ID NO: 71). Other suitable signal sequences include a human CD4 signal peptide, e.g., having the sequence MNRGVPFRHLLLVLQLALLPAAT (SEQ ID NO: 72), a mouse Ig kappa chain V-III region signal peptide, e.g., having the sequence METDTLLLWVLLLWVPGSTG (SEQ ID NO: 73), a mouse H-2Kb signal peptide, e.g., having the sequence MVPCTLLLLLAAALAPTQTRA (SEQ ID NO: 74), a human serum albumin signal peptide, e.g., having the sequence MKWVTFISLLFLFSSAYS (SEQ ID NO: 75), a human IL-2 signal peptide, e.g., having the sequence MYRMQLLSCIALSLALVTNS (SEQ ID NO: 76), a human HLA-A*02:01 signal peptide, e.g., having the sequence MAVMAPRTLLLLLSGALALTQTWA (SEQ ID NO: 77) and a human b2m signal peptide, e.g., having the sequence MSRSVALAVLALLSLSGLEA (SEQ ID NO: 78).

### Linkers

In some embodiments, a fusion protein of the present disclosure comprises a peptide linker. In certain embodiments, wherein the fusion protein is an IL-15/IL-15Rα fusion protein, the IL-15 and the IL-15Rα sushi domain are separated by a peptide linker. In certain embodiments, the peptide linker comprises any of the amino acid sequences shown in Table 1.

TABLE 1
Peptide Linkers

| | | SEQ |
|---|---|---|
| | | ID |
| Linker | Amino Acid Sequence | NO: |
| (G₂S)₁ linker | GGS | 19 |
| (G₂S)₂ linker | GGSGGS | 20 |
| (G₂S)₃ linker | GGSGGSGGS | 21 |
| (G₂S)₄ linker | GGSGGSGGSGGS | 22 |
| (G₂S)₅ linker | GGSGGSGGSGGSGGS | 23 |
| (G₃S)₁ linker | GGGS | 24 |
| (G₃S)₂ linker | GGGSGGGS | 25 |
| (G₃S)₃ linker | GGGSGGGSGGGS | 26 |
| (G₃S)₄ linker | GGGSGGGSGGGSGGGS | 27 |
| (G₃S)₅ linker | GGGSGGGSGGGSGGGSGGGS | 28 |
| (G₄S)₁ linker | GGGGS | 29 |
| (G₄S)₂ linker | GGGGSGGGGS | 30 |
| (G₄S)₃ linker | GGGGSGGGGSGGGGS | 31 |
| (G₄S)₄ linker | GGGGSGGGGSGGGGSGGGGS | 32 |
| (G₄S)₅ linker | GGGGSGGGGSGGGGSGGGGSGGG | 33 |
| | GS | |
| Whitlow linker | GSTSGSGKPGSGEGSTKG | 34 |
| (EAAAK)4 linker 2 | EAAAKEAAAKEAAAKEAAAK | 35 |
| IL-linker | SGGSGGGGSGGGSGGGGSLQ | 127 |

### Spacer Region

In some embodiments, a fusion protein of the present disclosure can also comprise a spacer region that links the extracellular antigen-binding domain to the transmembrane domain. The spacer region may be flexible enough to allow the antigen-binding domain to orient in different directions to facilitate antigen recognition. In some embodiments, the spacer region may be a hinge from a human protein. For example, the hinge may be a human Ig (immunoglobulin) hinge, including without limitation an IgG4 hinge, an IgG2 hinge, a CD8a hinge, or an IgD hinge. In some embodiments, the spacer region may comprise an IgG4 hinge, an IgG2 hinge, an IgD hinge, a CD28 hinge, a KIR2DS2 hinge, an LNGFR hinge, a MAG hinge, or a PDGFR-beta extracellular linker. In some embodiments, the spacer region is localized between the antigen-binding domain and the transmembrane domain. In some embodiments, a spacer region may comprise any of the amino acid sequences listed in Table 2, or an amino acid sequence that is at least 90%, at least 91%, at least 92%, at least 93%, at least 94%, at least 95%, at least 96%, at least 97%, at least 98%, or at least 99% identical to any of the amino acid sequences listed in Table 2. Exemplary nucleic acid sequences encoding the spacers in Table 2 are shown in Table 3.

TABLE 2
Spacer Amino Acid Sequences

| Amino Acid Sequence | SEQ ID NO: | Description |
|---|---|---|
| TTTPAPRPPTPAPTIALQPLSLRPEACRPAAGGAVHTRGL | 49 | CD8 hinge |
| DFACD | | |
| AAAIEVMYPPPYLDNEKSNGTIIHVKGKHLCPSPLFPGPS | 40 | CD28 hinge |
| KP | | |
| ESKYGPPCPSCP | 41 | IgG4 minimal hinge |
| ESKYGPPAPSAP | 42 | IgG4 minimal hinge, no |
| | | disulfides |
| ESKYGPPCPPCP | 43 | IgG4 S228P minimal hinge, |
| | | enhanced disulfide formation |
| EPKSCDKTHTCP | 44 | IgG1 minimal hinge |
| AAAFVPVFLPAKPTTTPAPRPPTPAPTIASQPLSLRPEAC | 45 | Extended CD8a hinge |
| RPAAGGAVHTRGLDFACDIYIWAPLAGTCGVLLLSLVIT | | |
| LYCNHRN | | |
| ACPTGLYTHSGECCKACNLGEGVAQPCGANQTVCEPCL | 46 | LNGFR hinge |
| DSVTFSDVVSATEPCKPCTECVGLQSMSAPCVEADDAV | | |
| CRCAYGYYQDETTGRCEACRVCEAGSGLVFSCQDKQN | | |
| TVCEECPDGTYSDEADAEC | | |
| ACPTGLYTHSGECCKACNLGEGVAQPCGANQTVC | 47 | Truncated LNGFR hinge |
| | | (TNFR-Cys1) |
| AVGQDTQEVIVVPHSLPFKV | 48 | PDGFR-beta extracellular |
| | | linker |
| YPPVIVEMNSSVEAIEGSHVSLLCGADSNPPPLLTWMRD | 3 | MAG hinge |
| GTVLREAVAESLLLELEEVTPAEDGVYACLAENAYGQD | | |
| NRTVGLSVMYAPWKPTVNGTMVAVEGETVSILCSTQS | | |
| NPDPILTIFKEKQILSTVIYESELQLELPAVSPEDDGEYWC | | |
| VAENQYGQRATAFNLSVEFAPVLLLESHCAAARDTVQC | | |
| LCVVKSNPEPSVAFELPSRNVTVNESEREFVYSERSGLV | | |
| LTSILTLRGQAQAPPRVICTARNLYGAKSLELPFQGAHR | | |
| LMWAKIGP | | |

In some embodiments, the spacer region comprises the sequence shown in SEQ ID NO:39. In some embodiments, the spacer region comprises the sequence shown in SEQ ID NO:40. In some embodiments, the spacer region comprises the sequence shown in SEQ ID NO:41. In some embodiments, the spacer region comprises the sequence shown in SEQ ID NO:42. In some embodiments, the spacer region comprises the sequence shown in SEQ ID NO:43. In some embodiments, the spacer region comprises the sequence shown in SEQ ID NO:44. In some embodiments, the spacer region comprises the sequence shown in SEQ ID NO:45. In some embodiments, the spacer region comprises the sequence shown in SEQ ID NO:46. In some embodiments, the spacer region comprises the sequence shown in SEQ ID NO:47. In some embodiments, the spacer region comprises the sequence shown in SEQ ID NO:48. In some embodiments, the spacer region comprises the sequence shown in SEQ ID NO:3.

In some embodiments, the spacer region comprises the sequence TTTPAPRPPTPAPTIALQPLSLRPEACRPAAGGAVHTRGLDFACD (SEQ ID NO:49). In some embodiments, the spacer region comprises the sequence ALSNSIMYFSHFVPVFLPAKPTTTPAPRPPTPAPTIASQPLSLRPEACRPAAGGAVHTR GLDFACD (SEQ ID NO:50). In some embodiments, the spacer region comprises the sequence FVPVFLPAKPTTTPAPRPPTPAPTIALQPLSLRPEACRPAAGGAVHTRGLDFACD (SEQ ID NO: 51).

In some embodiments, polynucleotides encoding any of the spacer regions of the present disclosure may comprise any of the nucleic acid sequences listed in Table 3, or a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 3.

TABLE 3
Spacer Nucleic Acid Sequences

| Nucleic Acid Sequence | SEQ ID NO: | Description |
|---|---|---|
| ACCACAACTCCTGCTCCTAGACCTCCTACACCAGCTC | 52 | CD8 hinge |
| CTACAATCGCCCTGCAGCCACTGTCTCTGAGGCCAGA | | |
| AGCTTGTAGACCTGCTGCAGGCGGAGCCGTGCATACA | | |
| AGAGGACTGGATTTCGCCTGCGAC | | |
| GCAGCAGCTATCGAGGTGATGTATCCTCCGCCCTACC | 53 | CD28 hinge |
| TGGATAATGAAAAGAGTAATGGGACTATCATTCATGT | | |
| AAAAGGGAAGCATCTTTGTCCTTCTCCCCTTTTCCCCG | | |
| GTCCGTCTAAACCT | | |
| GAAAGCAAGTACGGTCCACCTTGCCCTAGCTGTCCG | 54 | IgG4 minimal hinge |
| GAATCCAAGTACGGCCCCCCAGCGCCTAGTGCCCCA | 55 | IgG4 minimal hinge, no |
| | | disulfides |
| GAATCTAAATATGGCCCGCCATGCCCGCCTTGCCCA | 56 | IgG4 S228P minimal hinge, |
| | | enhanced disulfide formation |
| GAACCGAAGTCTTGTGATAAAACTCATACGTGCCCG | 57 | IgG1 minimal hinge |
| GCTGCTGCTTTCGTACCCGTGTTCCTCCCTGCTAAGCC | 58 | Extended CD8a hinge |
| TACGACTACCCCCGCACCGAGACCACCCACGCCAGC | | |
| ACCCACGATTGCTAGCCAGCCCCTTAGTTTGCGACCA | | |
| GAAGCTTGTCGGCCTGCTGCTGGTGGCGCGGTACATA | | |
| CCCGCGGCCTTGATTTTGCTTGCGATATATATATCTGG | | |
| GCGCCTCTGGCCGGAACATGCGGGGTCCTCCTCCTTT | | |
| CTCTGGTTATTACTCTCTACTGTAATCACAGGAAT | | |
| GCCTGCCCGACCGGGCTCTACACTCATAGCGGGGAAT | 59 | LNGFR hinge |
| GTTGTAAGGCATGTAACTTGGGTGAGGGCGTCGCACA | | |
| GCCCTGCGGAGCTAACCAAACAGTGTGCGAACCCTG | | |
| CCTCGATAGTGTGACGTTCTCTGATGTTGTATCAGCTA | | |
| CAGAGCCTTGCAAACCATGTACTGAGTGCGTTGGACT | | |
| TCAGTCAATGAGCGCTCCATGTGTGGAGGCAGATGAT | | |
| GCGGTCTGTCGATGTGCTTACGGATACTACCAAGACG | | |
| AGACAACAGGGCGGTGCGAGGCCTGTAGAGTTTGTG | | |
| AGGCGGGCTCCGGGCTGGTGTTTTCATGTCAAGACAA | | |
| GCAAAATACGGTCTGTGAAGAGTGCCCTGATGGCACC | | |
| TACTCAGACGAAGCAGATGCAGAATGC | | |
| GCCTGCCCTACAGGACTCTACACGCATAGCGGTGAGT | 60 | Truncated LNGFR hinge |
| GTTGTAAAGCATGCAACCTCGGGGAAGGTGTAGCCC | | (TNFR-Cys1) |
| AGCCATGCGGGGCTAACCAAACCGTTTGC | | |
| GCTGTGGGCCAGGACACGCAGGAGGTCATCGTGGTG | 61 | PDGFR-beta extracellular |
| CCACACTCCTTGCCCTTTAAGGTG | | linker |
| TACCCTCCAGTGATCGTGGAAATGAACAGCAGCGTGG | 4 | MAG hinge |
| AAGCCATCGAGGGCTCTCATGTGTCTCTGCTGTGTGG | | |
| CGCCGACAGCAATCCTCCTCCTCTGCTGACCTGGATG | | |
| AGAGATGGCACCGTGCTGAGAGAAGCCGTGGCCGAA | | |
| TCTCTGCTGCTGGAACTGGAAGAAGTGACCCCTGCCG | | |
| AGGATGGCGTGTACGCTTGTCTGGCCGAGAATGCCTA | | |
| CGGCCAGGACAATAGAACCGTGGGCCTGTCCGTGAT | | |
| GTACGCCCCTTGGAAGCCTACCGTGAACGGCACAATG | | |
| GTGGCCGTGGAAGGCGAGACAGTGTCCATCCTGTGTA | | |
| GCACCCAGAGCAACCCCGATCCTATCCTGACCATCTT | | |
| CAAAGAGAAGCAGATCCTGAGCACCGTGATCTACGA | | |
| GAGCGAACTGCAGCTCGAACTGCCCGCTGTGTCCCCA | | |
| GAGGATGATGGCGAATATTGGTGCGTGGCAGAGAAC | | |
| CAGTACGGCCAGAGAGCCACCGCCTTCAACCTGAGC | | |
| GTGGAATTTGCTCCCGTGCTGCTGCTCGAGAGCCATT | | |
| GTGCTGCCGCCAGAGATACCGTGCAGTGCCTGTGTGT | | |
| GGTCAAGTCTAACCCCGAGCCTAGCGTGGCCTTTGAG | | |
| CTGCCCAGCAGAAACGTGACCGTGAATGAGAGCGAG | | |
| CGCGAGTTCGTGTACAGCGAGAGATCTGGACTGGTGC | | |
| TGACCAGCATCCTGACACTGAGAGGACAGGCTCAGG | | |
| CCCCTCCTAGAGTGATCTGCACCGCCAGAAATCTGTA | | |
| CGGCGCCAAGAGCCTGGAACTGCCATTTCAGGGCGC | | |
| CCACAGACTCATGTGGGCCAAGATTGGACCT | | |

In some embodiments, a fusion protein of the present disclosure may further include a short oligopeptide or polypeptide linker that is between 2 amino acid residues and 10 amino acid residues in length, and that may form the linkage between the transmembrane domain and the cytoplasmic region of the fusion protein. A non-limiting example of a suitable linker is a glycine-serine doublet. In some embodiments, the linker comprises the ammo acid sequence of GGCKJSGGCKJS (SEQ ID NO:62).

### Transmembrane Domains

In some embodiments, the transmembrane domain of a fusion protein of the present disclosure comprises a hydrophobic alpha helix that spans at least a portion of a cell membrane. It has been shown that different transmembrane domains can result in different receptor stability. After antigen recognition, receptors cluster and a signal is transmitted to the cell. In some embodiments, the transmembrane domain of a fusion protein of the present disclosure can comprise the transmembrane domain of a CD8 polypeptide, a CD28 polypeptide, a CD3-zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a LIR-1 (LILRB1) polypeptide, or can be a synthetic peptide, or any combination thereof. In some embodiments, the transmembrane domain does not comprise the transmembrane domain of IL-15Rα.

In some embodiments, the transmembrane domain is derived from a CD8 polypeptide. Any suitable CD8 polypeptide may be used. Exemplary CD8 polypeptides include, without limitation, NCBI Reference Nos. NP_001139345 and AAA92533.1. Examples of CD8 transmembrane domains include IYIWAPLAGTCGVLLLSLVIT (SEQ ID NO: 36), IYIWAPLAGTCGVLLLSLVITLYCNHR (SEQ ID NO:37), and IYIWAPLAGTCGVLLLSLVITLYCNHRN (SEQ ID NO:38). In some embodiments, the transmembrane domain comprises the sequence IYIWAPLAGTCGVLLLSLVIT (SEQ ID NO: 36). In some embodiments, the transmembrane domain comprises the sequence IYIWAPLAGTCGVLLLSLVITLYCNHR (SEQ ID NO:37). In some embodiments, the transmembrane domain comprises the sequence IYIWAPLAGTCGVLLLSLVITLYCNHRN (SEQ ID NO:38).

In some embodiments, the transmembrane domain is derived from a CD28 polypeptide. Any suitable CD28 polypeptide may be used. Exemplary CD28 polypeptides include, without limitation, NCBI Reference Nos. NP_006130.1 and NP_031668.3. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1. In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide. Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. Any suitable LIR-1 (LILRB1) polypeptide may be used. Exemplary LIR-1 (LILRB 1) polypeptides include, without limitation, NCBI Reference Nos. NP_001075106.2 and NP_001075107.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2, or fragments thereof. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NG2C.

Transmembrane domain sequences suitable for use in fusion proteins of the present disclosure are provided in Table 4 below.

TABLE 4
Exemplary transmembrane domain sequences

| Amino Acid Sequence | Nucleotide sequence | Description |
|---|---|---|
| VAISTSTVLLCGLSAVSLLACYL | GTGGCCATCAGCACAAGCACCG | IL-15Rα transmembrane |
| (SEQ ID NO: 5) | TGCTGCTGTGTGGACTGTCTGCC | domain |
| | GTTTCTCTGCTGGCCTGCTACCT | |
| | G | |
| | (SEQ ID NO: 6) | |
| FWVLVVVGGVLACYSLLVTVAFIIF | TTCTGGGTGCTCGTGGTTGTTGG | CD28 transmembrane |
| WV | CGGAGTGCTGGCCTGTTACTCTC | domain |
| (SEQ ID NO: 7) | TGCTGGTCACCGTGGCCTTCATC | |
| | ATCTTTTGGGTC | |
| | (SEQ ID NO: 8) | |
| VAAILGLGLVLGLLGPLAILL | GTGGCCGCCATTCTTGGACTGG | OX40 transmembrane |
| (SEQ ID NO: 9) | GACTTGTTCTGGGACTGCTGGG | domain |
| | ACCTCTGGCCATTCTGCTG | |
| | (SEQ ID NO: 10) | |
| IYIWAPLAGTCGVLLLSLVIT | ATCTACATCTGGGCCCCTCTGGC | CD8 transmembrane |
| (SEQ ID NO: 36) | TGGAACATGCGGAGTGTTGCTG | domain |
| | CTGAGCCTGGTCATCACC | |
| | (SEQ ID NO: 11) | |
| MGLAFLVLVALVWFLVEDWLS | ATGGGCCTCGCCTTTCTGGTGCT | NKp46 transmembrane |
| (SEQ ID NO: 12) | GGTGGCCCTTGTGTGGTTCCTGG | domain |
| | TGGAAGATTGGCTGAGC | |
| | (SEQ ID NO: 13) | |
| FLVIIVILSALFLGTLACFCV | TTCCTGGTCATCATCGTGATCCT | 2B4 transmembrane |
| (SEQ ID NO: 14) | GAGCGCCCTGTTCCTGGGCACC | domain |
| | CTGGCCTGTTTTTGCGTG | |
| | (SEQ ID NO: 15) | |

In certain embodiments, a transmembrane domain of the present disclosure comprises the amino acid sequence of SEQ ID NO:5. In certain embodiments, a transmembrane domain of the present disclosure comprises the amino acid sequence of SEQ ID NO: 7. In certain embodiments, a transmembrane domain of the present disclosure comprises the amino acid sequence of SEQ ID NO:9. In certain embodiments, a transmembrane domain of the present disclosure comprises the amino acid sequence of SEQ ID NO: 36. In certain embodiments, a transmembrane domain of the present disclosure comprises the amino acid sequence of SEQ ID NO:12. In certain embodiments, a transmembrane domain of the present disclosure comprises the amino acid sequence of SEQ ID NO: 14.

### Intracellular Domains

In some embodiments, a fusion protein of the present disclosure comprises one or more cytoplasmic domains or regions. The cytoplasmic domain or region of the fusion protein may include an intracellular signaling domain. In some embodiments, wherein the fusion protein comprises an IL-15 in the extracellular domain, the fusion protein does not comprise the intracellular signaling domain of IL-15Rα.

Examples of suitable intracellular signaling domains that may be used in fusion proteins of the present disclosure include, without limitation, cytoplasmic sequences of the T cell receptor (TCR) and co-receptors that act in concert to modulate signal transduction following antigen receptor engagement, as well as any derivative or variant of these sequences and any recombinant sequence that has the same functional capability.

Without wishing to be bound by theory, it is believed that signals generated through the TCR alone are insufficient for full activation of the T cell and that a secondary and/or co-stimulatory signal is thus also typically used for full activation. Accordingly, T cell activation may be mediated by two distinct classes of cytoplasmic signaling sequences, those that initiate antigen-dependent primary activation through the TCR (primary intracellular signaling domains) and those that act in an antigen-independent manner to provide a secondary or co-stimulatory signal (secondary cytoplasmic domain, e.g., a co-stimulatory domain). In addition, T cell signaling and function (e.g., an activating signaling cascade) can be negatively regulated by inhibitory receptors present in a T cell through intracellular inhibitory co-signaling domains.

In some embodiments, the intracellular signaling domain of a fusion protein of the present disclosure can include an inhibitory intracellular signaling domains. In some embodiments, the inhibitory intracellular signaling domain includes one or more intracellular inhibitory co-signaling domains. In some embodiments, the one or more intracellular inhibitory co-signaling domains are linked to other domains (e.g., a transmembrane domain) through a peptide linker (e.g., see Table 1) or a spacer or hinge sequence (e.g., see Table 2). In some embodiments, when two or more intracellular inhibitory co-signaling domains are present, the two or more intracellular inhibitory co-signaling domains can be linked through a peptide linker (e.g., see Table 1) or a spacer or hinge sequence (e.g., see Table 2). In some embodiments, the intracellular inhibitory co-signaling domain is an inhibitory domain. In some embodiments, the one or more intracellular inhibitory co-signaling domains of a chimeric protein comprises one or more ITIM-containing protein, or fragment(s) thereof. ITIMs are conserved amino acid sequences found in cytoplasmic tails of many inhibitory immune receptors. In some embodiments, the one or more ITIM-containing protein, or fragments thereof, is selected from PD-1, CTLA4, TIGIT, BTLA, and LAIR1. In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAG3, HAVR, GITR, and PD-L1. The inhibitory intracellular signaling domain can further include an enzymatic inhibitory domain. In some embodiments, the enzymatic inhibitory domain comprises an enzyme catalytic domain. In some embodiments, the enzyme catalytic domain is derived from an enzyme including, but not limited to, CSK, SHP-1, PTEN, CD45, CD148, PTP-MEG1, PTP-PEST, c-CBL, CBL-b, PTPN22, LAR, PTPH1, SHIP-1, or RasGAP. Examples of enzymatic regulation of signaling is described in more detail in Pavel Otáhal et al. (Biochim Biophys Acta. 2011 February; 1813 (2): 367-76), Kosugi A., et al. (Involvement of SHP-1 tyrosine phosphatase in TCR-mediated signaling pathways in lipid rafts, Immunity, 2001 June; 14 (6): 669-80), and Stanford, et al. (Regulation of TCR signaling by tyrosine phosphatases: from immune homeostasis to autoimmunity, Immunology, 2012 September; 137 (1): 1-19), each of which is incorporated herein by reference for all purposes.

In some embodiments, the intracellular signaling domain of a fusion protein of the present disclosure can comprise a primary signaling domain regulates primary activation of the TCR complex either in a stimulatory way or in an inhibitory way. Primary intracellular signaling domains that act in a stimulatory manner may contain signaling motifs which are known as immunoreceptor tyrosine-based activation motifs (ITAMs). Examples of suitable ITAM-containing primary intracellular signaling domains that that may be used in the CARs of the present disclosure include, without limitation, those of CD3-zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d.

In some embodiments, a fusion protein of the present disclosure comprises an intracellular signaling domain, e.g., a primary signaling domain of CD3-zeta polypeptide. A CD3-zeta polypeptide of the present disclosure may have an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_932170 or NP_001106864.2, or fragments thereof. In some embodiments, the CD3-zeta polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_932170 or NP_001106864.2 that is at least 20, at least 30, at least 40, at least 50, at least 60, at least 70, at least 80, at least 90, at least 100, at least 110, at least 120, at least 130, at least 140, at least 150, or at least 160, at least 170, or at least 180 amino acids in length.

Exemplary intracellular signaling domains of the present disclosure are provided in Table 4. Exemplary sequences of ICDs suitable for use in proteins of the present disclosure.

| Amino Acid Sequence | Nucleotide Sequence | Description |
|---|---|---|
| KSRQTPPLASVEMEAME | AAGTCCAGACAGACACCTCCTCTGGCCAGCGTGGAAATG | IL-15Rα ICD |
| ALPVTWGTSSRDEDLEN | GAAGCCATGGAAGCTCTGCCTGTGACCTGGGGCACCAGC | |
| CSHHL | TCCAGAGATGAGGACCTGGAAAACTGCTCCCACCACCTG | |
| (SEQ ID NO: 16) | (SEQ ID NO: 17) | |
| RSKRSRLLHSDYMNMTP | CGAAGCAAGCGGAGCCGGCTGCTGCACAGCGACTACAT | CD28 ICD |
| RRPGPTRKHYQPYAPPR | GAACATGACCCCTAGACGGCCCGGACCTACCAGAAAGC | |
| DFAAYRS | ACTACCAGCCTTACGCTCCTCCTAGAGATTTCGCCGCCT | |
| (SEQ ID NO: 18) | ACCGGTCC | |
| | (SEQ ID NO: 63) | |
| ALYLLRRDQRLPPDAHK | GCTCTGTACCTGCTGAGAAGGGACCAGAGACTGCCTCCT | OX40 ICD |
| PPGGGSFRTPIQEEQADA | GACGCTCACAAACCTCCAGGCGGAGGCAGCTTCAGAAC | |
| HSTLAKI | CCCTATCCAAGAGGAACAGGCTGACGCCCACAGCACCCT | |
| (SEQ ID NO: 64) | GGCCAAGATC | |
| | (SEQ ID NO: 65) | |
| KRGRKKLLYIFKQPFMR | AAGCGGGGCAGAAAGAAGCTGCTGTACATCTTCAAGCA | 4-1BB ICD |
| PVQTTQEEDGCSCRFPE | GCCCTTCATGCGGCCCGTGCAGACCACACAAGAGGAAG | |
| EEEGGCEL | ATGGCTGCTCCTGCAGATTCCCCGAGGAAGAAGAAGGC | |
| (SEQ ID NO: 66) | GGCTGCGAGCTG | |
| | (SEQ ID NO: 128) | |
| RKRTRERASRASTWEGR | CGGAAGCGGACAAGAGAGAGAGCCAGCAGAGCCTCTAC | Nkp46 ICD |
| RRLNTQTL | CTGGGAGGGAAGAAGAAGGCTGAACACCCAGACACTC | |
| (SEQ ID NO: 67) | (SEQ ID NO: 68) | |
| WRRKRKEKQSETSPKEF | TGGCGCCGCAAGCGGAAAGAGAAGCAGTCTGAGACAAG | 2B4 ICD |
| LTIYEDVKDLKTRRNHE | CCCCAAAGAGTTCCTGACCATCTACGAGGACGTGAAGGA | |
| QEQTFPGGGSTIYSMIQS | CCTGAAAACCCGGCGGAACCACGAGCAAGAGCAGACCT | |
| QSSAPTSQEPAYTLYSLI | TTCCTGGCGGCGGAAGCACCATCTACAGCATGATCCAGA | |
| QPSRKSGSRKRNHSPSF | GCCAGTCTAGCGCCCCTACCAGCCAAGAGCCTGCCTACA | |
| NSTIYEVIGKSQPKAQNP | CACTGTACTCCCTGATCCAGCCTAGCAGAAAGAGCGGCA | |
| ARLSRKELENFDVYS | GCCGGAAGAGAAATCACAGCCCCAGCTTCAACAGCACG | |
| (SEQ ID NO: 69) | ATCTACGAAGTGATCGGCAAGAGCCAGCCAAAGGCTCA | |
| | GAACCCTGCCAGGCTGAGCCGGAAAGAGCTGGAAAACT | |
| | TCGACGTGTACAGC | |
| | (SEQ ID NO: 105) | |

In other embodiments, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In one embodiment, a primary signaling domain comprises one, two, three, four or more ITAM motifs.

In some embodiments, the intracellular signaling domain of a fusion protein of the present disclosure can comprise the CD3-zeta signaling domain by itself, or it can be combined with any other desired intracellular signaling domain(s) useful in the context of a fusion protein of the present disclosure. For example, the intracellular signaling domain of the fusion protein can comprise a CD3-zeta chain portion and a costimulatory signaling domain. The costimulatory signaling domain may refer to a portion of the fusion protein comprising the intracellular domain of a costimulatory molecule. A costimulatory molecule of the present disclosure is a cell surface molecule other than an antigen receptor or its ligands that may be used for an efficient response of lymphocytes to an antigen. Examples of suitable costimulatory molecules include, without limitation, CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and the like.

In some embodiments, the intracellular signaling sequences within the cytoplasmic portion of a fusion protein of the present disclosure may be linked to each other in a random or specified order. In some embodiments, a short oligopeptide or polypeptide linker, for example, between 2 amino acids and 10 amino acids (e.g., 2 amino acids, 3 amino acids, 4 amino acids, 5 amino acids, 6 amino acids, 7 amino acids, 8 amino acids, 9 amino acids, or 10 amino acids) in length may form the linkage between intracellular signaling sequences. In one embodiment, a glycine-serine doublet can be used as a suitable linker. In one embodiment, a single ammo acid, e.g., an alanine or a glycine, can be used as a suitable linker.

In some embodiments, the intracellular signaling domain comprises two or more costimulatory signaling domains, e.g., two costimulatory signaling domains, three costimulatory signaling domains, four costimulatory signaling domains, five costimulatory signaling domains, six costimulatory signaling domains, seven costimulatory signaling domains, eight costimulatory signaling domains, nine costimulatory signaling domains, 10 costimulatory signaling domains, or more costimulatory signaling domains. In one embodiment, the intracellular signaling domain comprises two costimulatory signaling domains. In some embodiments, the two or more costimulatory signaling domains are separated by a linker of the present disclosure (e.g., any of the linkers described in Table 1). In one embodiment, the linker is a glycine residue. In another embodiment, the linker is an alanine residue.

In some embodiments, a fusion protein of the present disclosure comprises the amino acid sequence of IL-15 and an IL-15Rα sushi domain, connected by a linker, e.g., a linker disclosed in Table 1. In some embodiments, a fusion protein of the present disclosure comprises the amino acid sequence of:

(SEQ ID NO: 85)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTSSGGSGGGGSGGGSGGGGSLQITCPPPMSVEHAD
IWVKSYSLYSRERYICNSGFKRKAGTSSLTECVLNKATNVAHWTTPSLK
CIR.

In some embodiments, the ECD does not comprise a non-sushi domain of IL-15Rα. In some embodiments, the ECD further comprises a non-sushi domain of IL-15Rα. In certain embodiments, the ECD further comprises the amino acid sequence of

(SEQ ID NO: 104)
DPALVHQRPAPPSTVTTAGVTPQPESLSPSGKEPAASSPSSNNTAATTA
AIVPGSQLMPSKSPSTGTTEISSHESSHGTPSQTTAKNWELTASASHQP
PGVYPQGHSDTT.

In some embodiments, a fusion protein described herein comprises, from N-terminus to C-terminus, a signal sequence, an ECD, a hinge, a transmembrane domain, and an ICD. In certain embodiments, a fusion protein of the present disclosure comprises, from N-terminus to C-terminus, an IgE signal sequence, an ECD, a hinge, a transmembrane domain, and the ICD.

Exemplary amino acid sequences of fusion proteins described herein are provided in Table 5 below, and exemplary nucleic acid sequences are provided in Table 6.

TABLE 5
Exemplary fusion proteins of the present disclosure.

| Fusion Protein | Amino Acid Sequence | Description |
|---|---|---|
| SB05497 | MDWTWILFLVAAATRVHSNWVNVISDLKKIEDLIQS | IgEss-IL-15-linker- |
| | MHIDATLYTESDVHPSCKVTAMKCFLLELQVISLESG | IL15RA(sushi)- |
| | DASIHDTVENLIILANNSLSSNGNVTESGCKECEELEE | IL15RA(NSECD)-- |
| | KNIKEFLQSFVHIVQMFINTSSGGSGGGGSGGGSGGG | IL15RA(TM)- |
| | GSLQITCPPPMSVEHADIWVKSYSLYSRERYICNSGFK | IL15RA(ICD) |
| | RKAGTSSLTECVLNKATNVAHWTTPSLKCIRDPALVH | |
| | QRPAPPSTVTTAGVTPQPESLSPSGKEPAASSPSSNNTA | |
| | ATTAAIVPGSQLMPSKSPSTGTTEISSHESSHGTPSQTT | |
| | AKNWELTASASHQPPGVYPQGHSDTTVAISTSTVLLC | |
| | GLSAVSLLACYLKSRQTPPLASVEMEAMEALPVTWG | |
| | TSSRDEDLENCSHHL | |
| | (SEQ ID NO: 86) | |
| SB05498 | MDWTWILFLVAAATRVHSNWVNVISDLKKIEDLIQS | IgEss-IL-15-linker- |
| | MHIDATLYTESDVHPSCKVTAMKCFLLELQVISLESG | IL15RA(sushi)- |
| | DASIHDTVENLIILANNSLSSNGNVTESGCKECEELEE | IL15RA(NSECD)-- |
| | KNIKEFLQSFVHIVQMFINTSSGGSGGGGSGGGSGGG | IL15RA(TM)-CD28 |
| | GSLQITCPPPMSVEHADIWVKSYSLYSRERYICNSGFK | |
| | RKAGTSSLTECVLNKATNVAHWTTPSLKCIRDPALVH | |
| | QRPAPPSTVTTAGVTPQPESLSPSGKEPAASSPSSNNTA | |
| | ATTAAIVPGSQLMPSKSPSTGTTEISSHESSHGTPSQTT | |
| | AKNWELTASASHQPPGVYPQGHSDTTVAISTSTVLLC | |
| | GLSAVSLLACYLRSKRSRLLHSDYMNMTPRRPGPTRK | |
| | HYQPYAPPRDFAAYRS | |
| | (SEQ ID NO: 88) | |
| SB05499 | MDWTWILFLVAAATRVHSNWVNVISDLKKIEDLIQS | IgEss-IL-15-linker- |
| | MHIDATLYTESDVHPSCKVTAMKCFLLELQVISLESG | IL15RA(sushi)- |
| | DASIHDTVENLIILANNSLSSNGNVTESGCKECEELEE | IL15RA(NSECD)- |
| | KNIKEFLQSFVHIVQMFINTSSGGSGGGGSGGGSGGG | CD28-CD28-CD28 |
| | GSLQITCPPPMSVEHADIWVKSYSLYSRERYICNSGFK | |
| | RKAGTSSLTECVLNKATNVAHWTTPSLKCIR | |
| | DPALVHQRPAPPSTVTTAGVTPQPESLSPSGKEPAASS | |
| | PSSNNTAATTAAIVPGSQLMPSKSPSTGTTEISSHESSH | |
| | GTPSQTTAKNWELTASASHQPPGVYPQGHSDTTAAAI | |
| | EVMYPPPYLDNEKSNGTIIHVKGKHLCPSPLFPGPSKP | |
| | FWVLVVVGGVLACYSLLVTVAFIIFWVRSKRSRLLHS | |
| | DYMNMTPRRPGPTRKHYQPYAPPRDFAAYRS | |
| | (SEQ ID NO: 89) | |
| SB05500 | MDWTWILFLVAAATRVHSNWVNVISDLKKIEDLIQS | IgEss-IL-15-linker- |
| | MHIDATLYTESDVHPSCKVTAMKCFLLELQVISLESG | IL15RA(sushi)- |
| | DASIHDTVENLIILANNSLSSNGNVTESGCKECEELEE | IL15RA(NSECD)- |
| | KNIKEFLQSFVHIVQMFINTSSGGSGGGGSGGGSGGG | CD8-CD28-CD28 |
| | GSLQITCPPPMSVEHADIWVKSYSLYSRERYICNSGFK | |
| | RKAGTSSLTECVLNKATNVAHWTTPSLKCIR | |
| | TTTPAPRPPTPAPTIALQPLSLRPEACRPAAGGAVHTR | |
| | GLDFACDFWVLVVVGGVLACYSLLVTVAFIIFWV | |
| | RSKRSRLLHSDYMNMTPRRPGPTRKHYQPYAPPRDFA | |
| | AYRS | |
| | (SEQ ID NO: 90) | |
| SB05501 | MDWTWILFLVAAATRVHSNWVNVISDLKKIEDLIQS | IgEss-IL-15-linker- |
| | MHIDATLYTESDVHPSCKVTAMKCFLLELQVISLESG | IL15RA(sushi)--CD28- |
| | DASIHDTVENLIILANNSLSSNGNVTESGCKECEELEE | CD28-CD28 |
| | KNIKEFLQSFVHIVQMFINTSSGGSGGGGSGGGSGGG | |
| | GSLQITCPPPMSVEHADIWVKSYSLYSRERYICNSGFK | |
| | RKAGTSSLTECVLNKATNVAHWTTPSLKCIR | |
| | AAAIEVMYPPPYLDNEKSNGTIIHVKGKHLCPSPLFPG | |
| | PSKPFWVLVVVGGVLACYSLLVTVAFIIFWVRSKRSR | |
| | LLHSDYMNMTPRRPGPTRKHYQPYAPPRDFAAYRS | |
| | (SEQ ID NO: 91) | |
| SB05502 | MDWTWILFLVAAATRVHSNWVNVISDLKKIEDLIQS | IgEss-IL-15-linker- |
| | MHIDATLYTESDVHPSCKVTAMKCFLLELQVISLESG | IL15RA(sushi)--CD8- |
| | DASIHDTVENLIILANNSLSSNGNVTESGCKECEELEE | CD28-CD28 |
| | KNIKEFLQSFVHIVQMFINTSSGGSGGGGSGGGSGGG | |
| | GSLQITCPPPMSVEHADIWVKSYSLYSRERYICNSGFK | |
| | RKAGTSSLTECVLNKATNVAHWTTPSLKCIR | |
| | TTTPAPRPPTPAPTIALQPLSLRPEACRPAAGGAVHTR | |
| | GLDFACDFWVLVVVGGVLACYSLLVTVAFIIFWV | |
| | RSKRSRLLHSDYMNMTPRRPGPTRKHYQPYAPPRDFA | |
| | AYRS | |
| | (SEQ ID NO: 92) | |
| SB05917 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD28(H)-CD28(TM)- |
| | KNIKEFLQSFVHIVQMFINTSSGGSGGGGSGGGSGGG | (no-ICD) |
| | GSLQITCPPPMSVEHADIWVKSYSLYSRERYICNSGFK | |
| | RKAGTSSLTECVLNKATNVAHWTTPSLKCIR | |
| | AAAIEVMYPPPYLDNEKSNGTIIHVKGKHLCPSPLFPG | |
| | PSKPFWVLVVVGGVLACYSLLVTVAFIIFWV | |
| | (SEQ ID NO: 93) | |
| SB05918 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD28(H)-CD28(TM)- |
| | KNIKEFLQSFVHIVQMFINTSSGGSGGGGSGGGSGGG | IL15RA(ICD) |
| | GSLQITCPPPMSVEHADIWVKSYSLYSRERYICNSGFK | |
| | RKAGTSSLTECVLNKATNVAHWTTPSLKCIR | |
| | AAAIEVMYPPPYLDNEKSNGTIIHVKGKHLCPSPLFPG | |
| | PSKPFWVLVVVGGVLACYSLLVTVAFIIFWVKSRQTP | |
| | PLASVEMEAMEALPVTWGTSSRDEDLENCSHHL | |
| | (SEQ ID NO: 94) | |
| SB05919 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | MAG(H)-CD28(TM)- |
| | KNIKEFLQSFVHIVQMFINTSSGGSGGGGSGGGSGGG | CD28(ICD) |
| | GSLQITCPPPMSVEHADIWVKSYSLYSRERYICNSGFK | |
| | RKAGTSSLTECVLNKATNVAHWTTPSLKCIRYPPVIVE | |
| | MNSSVEAIEGSHVSLLCGADSNPPPLLTWMRDGTVLR | |
| | EAVAESLLLELEEVTPAEDGVYACLAENAYGQDNRT | |
| | VGLSVMYAPWKPTVNGTMVAVEGETVSILCSTQSNP | |
| | DPILTIFKEKQILSTVIYESELQLELPAVSPEDDGEYWC | |
| | VAENQYGQRATAFNLSVEFAPVLLLESHCAAARDTV | |
| | QCLCVVKSNPEPSVAFELPSRNVTVNESEREFVYSERS | |
| | GLVLTSILTLRGQAQAPPRVICTARNLYGAKSLELPFQ | |
| | GAHRLMWAKIGPFWVLVVVGGVLACYSLLVTVAFIIF | |
| | WVRSKRSRLLHSDYMNMTPRRPGPTRKHYQPYAPPR | |
| | DFAAYRS | |
| | (SEQ ID NO: 95) | |
| SB05920 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD8(H)-OX40(TM)- |
| | KNIKEFLQSFVHIVQMFINTSSGGSGGGGSGGGSGGG | OX40(ICD) |
| | GSLQITCPPPMSVEHADIWVKSYSLYSRERYICNSGFK | |
| | RKAGTSSLTECVLNKATNVAHWTTPSLKCIRTTTPAP | |
| | RPPTPAPTIALQPLSLRPEACRPAAGGAVHTRGLDFAC | |
| | DVAAILGLGLVLGLLGPLAILLALYLLRRDQRLPPDAH | |
| | KPPGGGSFRTPIQEEQADAHSTLAKI | |
| | (SEQ ID NO: 96) | |
| SB05921 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD8(H)-CD8(TM)-4- |
| | KNIKEFLQSFVHIVQMFINTSSGGSGGGGSGGGSGGG | 1BB(ICD) |
| | GSLQITCPPPMSVEHADIWVKSYSLYSRERYICNSGFK | |
| | RKAGTSSLTECVLNKATNVAHWTTPSLKCIR | |
| | TTTPAPRPPTPAPTIALQPLSLRPEACRPAAGGAVHTR | |
| | GLDFACDIYIWAPLAGTCGVLLLSLVITKRGRKKLLYI | |
| | FKQPFMRPVQTTQEEDGCSCRFPEEEEGGCEL | |
| | (SEQ ID NO: 97) | |
| SB05922 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD8(H)-NKp46(TM)- |
| | KNIKEFLQSFVHIVQMFINTSSGGSGGGGSGGGSGGG | NKp46(ICD) |
| | GSLQITCPPPMSVEHADIWVKSYSLYSRERYICNSGFK | |
| | RKAGTSSLTECVLNKATNVAHWTTPSLKCIRTTTPAP | |
| | RPPTPAPTIALQPLSLRPEACRPAAGGAVHTRGLDFAC | |
| | DMGLAFLVLVALVWFLVEDWLSRKRTRERASRAST | |
| | WEGRRRLNTQTL | |
| | (SEQ ID NO: 98) | |
| SB05923 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD8(H)-2B4(TM)- |
| | KNIKEFLQSFVHIVQMFINTSSGGSGGGGSGGGSGGG | 2B4(ICD) |
| | GSLQITCPPPMSVEHADIWVKSYSLYSRERYICNSGFK | |
| | RKAGTSSLTECVLNKATNVAHWTTPSLKCIR | |
| | TTTPAPRPPTPAPTIALQPLSLRPEACRPAAGGAVHTR | |
| | GLDFACDFLVIIVILSALFLGTLACFCVWRRKRKEKQS | |
| | ETSPKEFLTIYEDVKDLKTRRNHEQEQTFPGGGSTIYS | |
| | MIQSQSSAPTSQEPAYTLYSLIQPSRKSGSRKRNHSPSF | |
| | NSTIYEVIGKSQPKAQNPARLSRKELENFDVYS | |
| | (SEQ ID NO: 99) | |
| SB05924 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD28(H)-CD28(TM)- |
| | KNIKEFLQSFVHIVQMFINTSSGGSGGGGSGGGSGGG | OX40(ICD) |
| | GSLQITCPPPMSVEHADIWVKSYSLYSRERYICNSGFK | |
| | RKAGTSSLTECVLNKATNVAHWTTPSLKCIR | |
| | AAAIEVMYPPPYLDNEKSNGTIIHVKGKHLCPSPLFPG | |
| | PSKPFWVLVVVGGVLACYSLLVTVAFIIFWVALYLLR | |
| | RDQRLPPDAHKPPGGGSFRTPIQEEQADAHSTLAKI | |
| | (SEQ ID NO: 100) | |
| SB05925 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD28(H)-CD28(TM)- |
| | KNIKEFLQSFVHIVQMFINTSSGGSGGGGSGGGSGGG | 4-1BB(ICD) |
| | GSLQITCPPPMSVEHADIWVKSYSLYSRERYICNSGFK | |
| | RKAGTSSLTECVLNKATNVAHWTTPSLKCIR | |
| | AAAIEVMYPPPYLDNEKSNGTIIHVKGKHLCPSPLFPG | |
| | PSKPFWVLVVVGGVLACYSLLVTVAFIIFWVKRGRKK | |
| | LLYIFKQPFMRPVQTTQEEDGCSCRFPEEEEGGCEL | |
| | (SEQ ID NO: 101) | |
| SB05926 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD28(H)-CD28(TM)- |
| | KNIKEFLQSFVHIVQMFINTSSGGSGGGGSGGGSGGG | NKp46(ICD) |
| | GSLQITCPPPMSVEHADIWVKSYSLYSRERYICNSGFK | |
| | RKAGTSSLTECVLNKATNVAHWTTPSLKCIR | |
| | AAAIEVMYPPPYLDNEKSNGTIIHVKGKHLCPSPLFPG | |
| | PSKPFWVLVVVGGVLACYSLLVTVAFIIFWVRKRTRE | |
| | RASRASTWEGRRRLNTQTL | |
| | (SEQ ID NO: 102) | |
| SB05927 | MDWTWILFLVAAATRVHSNWVNVISDLKKIEDLIQS | IgE(SS)-IL-15-linker- |
| | MHIDATLYTESDVHPSCKVTAMKCFLLELQVISLESG | IL15RA(sushi)-- |
| | DASIHDTVENLIILANNSLSSNGNVTESGCKECEELEE | CD28(H)-CD28(TM)- |
| | KNIKEFLQSFVHIVQMFINTSSGGSGGGGSGGGSGGG | 2B4(ICD) |
| | GSLQITCPPPMSVEHADIWVKSYSLYSRERYICNSGFK | |
| | RKAGTSSLTECVLNKATNVAHWTTPSLKCIRAAAIEV | |
| | MYPPPYLDNEKSNGTIIHVKGKHLCPSPLFPGPSKPFW | |
| | VLVVVGGVLACYSLLVTVAFIIFWVWRRKRKEKQSE | |
| | TSPKEFLTIYEDVKDLKTRRNHEQEQTFPGGGSTIYSM | |
| | IQSQSSAPTSQEPAYTLYSLIQPSRKSGSRKRNHSPSFN | |
| | STIYEVIGKSQPKAQNPARLSRKELENFDVYS | |
| | (SEQ ID NO: 103) | |

TABLE 6
Exemplary nucleic acids encoding fusion proteins of the present disclosure.

| Fusion Protein | Amino Acid Sequence | Description |
|---|---|---|
| SB05497 | ATGGACTGGACTTGGATACTCTTTCTGGTCGCTGCC | IgEss-IL-15-linker- |
| | GCCACACGGGTGCACTCTAATTGGGTCAACGTGATC | IL15RA(sushi)- |
| | AGCGACCTGAAGAAGATCGAGGACCTGATCCAGAG | IL15RA(NSECD)-- |
| | CATGCACATCGACGCCACACTGTACACCGAGAGCG | IL15RA(TM)- |
| | ACGTGCACCCTAGCTGTAAAGTGACCGCCATGAAG | IL15RA(ICD) |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGATCCCGCTCTGGTGCATC | |
| | AGCGTCCTGCTCCTCCAAGCACAGTGACAACAGCT | |
| | GGCGTGACACCCCAGCCTGAGTCTCTGAGCCCTTCT | |
| | GGAAAAGAGCCTGCCGCCAGCTCTCCCAGCTCTAA | |
| | CAATACTGCCGCCACCACAGCCGCCATCGTGCCTGG | |
| | ATCTCAACTGATGCCTAGCAAGAGCCCTAGCACCG | |
| | GCACCACAGAGATCAGCTCTCACGAGTCTAGCCAC | |
| | GGCACCCCTTCTCAGACCACCGCCAAGAATTGGGA | |
| | GCTGACAGCCAGCGCCTCTCATCAACCACCTGGCGT | |
| | GTACCCTCAGGGCCACAGCGATACAACAGTGGCCA | |
| | TCAGCACAAGCACCGTGCTGCTGTGTGGACTGTCTG | |
| | CCGTTTCTCTGCTGGCCTGCTACCTGAAGTCCAGAC | |
| | AGACACCTCCTCTGGCCAGCGTGGAAATGGAAGCC | |
| | ATGGAAGCTCTGCCTGTGACCTGGGGCACCAGCTCC | |
| | AGAGATGAGGACCTGGAAAACTGCTCCCACCACCT | |
| | G | |
| | (SEQ ID NO: 106) | |
| SB05498 | ATGGACTGGACTTGGATACTCTTTCTGGTCGCTGCC | IgEss-IL-15-linker- |
| | GCCACACGGGTGCACTCTAATTGGGTCAACGTGATC | IL15RA(sushi)- |
| | AGCGACCTGAAGAAGATCGAGGACCTGATCCAGAG | IL15RA(NSECD)-- |
| | CATGCACATCGACGCCACACTGTACACCGAGAGCG | IL15RA(TM)-CD28 |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGATCCCGCTCTGGTGCATC | |
| | AGCGTCCTGCTCCTCCAAGCACAGTGACAACAGCT | |
| | GGCGTGACACCCCAGCCTGAGTCTCTGAGCCCTTCT | |
| | GGAAAAGAGCCTGCCGCCAGCTCTCCCAGCTCTAA | |
| | CAATACTGCCGCCACCACAGCCGCCATCGTGCCTGG | |
| | ATCTCAACTGATGCCTAGCAAGAGCCCTAGCACCG | |
| | GCACCACAGAGATCAGCTCTCACGAGTCTAGCCAC | |
| | GGCACCCCTTCTCAGACCACCGCCAAGAATTGGGA | |
| | GCTGACAGCCAGCGCCTCTCATCAACCACCTGGCGT | |
| | GTACCCTCAGGGCCACAGCGATACAACAGTGGCCA | |
| | TCAGCACAAGCACCGTGCTGCTGTGTGGACTGTCTG | |
| | CCGTTTCTCTGCTGGCCTGCTACCTGCGAAGCAAGC | |
| | GGAGCCGGCTGCTGCACAGCGACTACATGAACATG | |
| | ACCCCTAGACGGCCCGGACCTACCAGAAAGCACTA | |
| | CCAGCCTTACGCTCCTCCTAGAGATTTCGCCGCCTA | |
| | CCGGTCC | |
| | (SEQ ID NO: 107) | |
| SB05499 | ATGGACTGGACTTGGATACTCTTTCTGGTCGCTGCC | IgEss-IL-15-linker- |
| | GCCACACGGGTGCACTCTAATTGGGTCAACGTGATC | IL15RA(sushi)- |
| | AGCGACCTGAAGAAGATCGAGGACCTGATCCAGAG | IL15RA(NSECD)- |
| | CATGCACATCGACGCCACACTGTACACCGAGAGCG | CD28-CD28-CD28 |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGATCCCGCTCTGGTGCATC | |
| | AGCGTCCTGCTCCTCCAAGCACAGTGACAACAGCT | |
| | GGCGTGACACCCCAGCCTGAGTCTCTGAGCCCTTCT | |
| | GGAAAAGAGCCTGCCGCCAGCTCTCCCAGCTCTAA | |
| | CAATACTGCCGCCACCACAGCCGCCATCGTGCCTGG | |
| | ATCTCAACTGATGCCTAGCAAGAGCCCTAGCACCG | |
| | GCACCACAGAGATCAGCTCTCACGAGTCTAGCCAC | |
| | GGCACCCCTTCTCAGACCACCGCCAAGAATTGGGA | |
| | GCTGACAGCCAGCGCCTCTCATCAACCACCTGGCGT | |
| | GTACCCTCAGGGCCACAGCGATACAACAGCAGCAG | |
| | CTATCGAGGTGATGTATCCTCCGCCCTACCTGGATA | |
| | ATGAAAAGAGTAATGGGACTATCATTCATGTAAAA | |
| | GGGAAGCATCTTTGTCCTTCTCCCCTTTTCCCCGGTC | |
| | CGTCTAAACCTTTCTGGGTGCTCGTGGTTGTTGGCG | |
| | GAGTGCTGGCCTGTTACTCTCTGCTGGTCACCGTGG | |
| | CCTTCATCATCTTTTGGGTCCGAAGCAAGCGGAGCC | |
| | GGCTGCTGCACAGCGACTACATGAACATGACCCCT | |
| | AGACGGCCCGGACCTACCAGAAAGCACTACCAGCC | |
| | TTACGCTCCTCCTAGAGATTTCGCCGCCTACCGGTC | |
| | C | |
| | (SEQ ID NO: 108) | |
| SB05500 | ATGGACTGGACTTGGATACTCTTTCTGGTCGCTGCC | IgEss-IL-15-linker- |
| | GCCACACGGGTGCACTCTAATTGGGTCAACGTGATC | IL15RA(sushi)- |
| | AGCGACCTGAAGAAGATCGAGGACCTGATCCAGAG | IL15RA(NSECD)- |
| | CATGCACATCGACGCCACACTGTACACCGAGAGCG | CD8-CD28-CD28 |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGATCCCGCTCTGGTGCATC | |
| | AGCGTCCTGCTCCTCCAAGCACAGTGACAACAGCT | |
| | GGCGTGACACCCCAGCCTGAGTCTCTGAGCCCTTCT | |
| | GGAAAAGAGCCTGCCGCCAGCTCTCCCAGCTCTAA | |
| | CAATACTGCCGCCACCACAGCCGCCATCGTGCCTGG | |
| | ATCTCAACTGATGCCTAGCAAGAGCCCTAGCACCG | |
| | GCACCACAGAGATCAGCTCTCACGAGTCTAGCCAC | |
| | GGCACCCCTTCTCAGACCACCGCCAAGAATTGGGA | |
| | GCTGACAGCCAGCGCCTCTCATCAACCACCTGGCGT | |
| | GTACCCTCAGGGCCACAGCGATACAACAACCACAA | |
| | CTCCTGCTCCTAGACCTCCTACACCAGCTCCTACAA | |
| | TCGCCCTGCAGCCACTGTCTCTGAGGCCAGAAGCTT | |
| | GTAGACCTGCTGCAGGCGGAGCCGTGCATACAAGA | |
| | GGACTGGATTTCGCCTGCGACTTCTGGGTGCTCGTG | |
| | GTTGTTGGCGGAGTGCTGGCCTGTTACTCTCTGCTG | |
| | GTCACCGTGGCCTTCATCATCTTTTGGGTCCGAAGC | |
| | AAGCGGAGCCGGCTGCTGCACAGCGACTACATGAA | |
| | CATGACCCCTAGACGGCCCGGACCTACCAGAAAGC | |
| | ACTACCAGCCTTACGCTCCTCCTAGAGATTTCGCCG | |
| | CCTACCGGTCC | |
| | (SEQ ID NO: 109) | |
| SB05501 | ATGGACTGGACTTGGATACTCTTTCTGGTCGCTGCC | IgEss-IL-15-linker- |
| | GCCACACGGGTGCACTCTAATTGGGTCAACGTGATC | IL15RA(sushi)--CD28- |
| | AGCGACCTGAAGAAGATCGAGGACCTGATCCAGAG | CD28-CD28 |
| | CATGCACATCGACGCCACACTGTACACCGAGAGCG | |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTCCGAAGCAAGCGGAGCCGGCTGCTGCAC | |
| | AGCGACTACATGAACATGACCCCTAGACGGCCCGG | |
| | ACCTACCAGAAAGCACTACCAGCCTTACGCTCCTCC | |
| | TAGAGATTTCGCCGCCTACCGGTCC | |
| | (SEQ ID NO: 110) | |
| SB05502 | ATGGACTGGACTTGGATACTCTTTCTGGTCGCTGCC | IgEss-IL-15-linker- |
| | GCCACACGGGTGCACTCTAATTGGGTCAACGTGATC | IL15RA(sushi)--CD8- |
| | AGCGACCTGAAGAAGATCGAGGACCTGATCCAGAG | CD28-CD28 |
| | CATGCACATCGACGCCACACTGTACACCGAGAGCG | |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAACCACAACTCCTGCTCCTA | |
| | GACCTCCTACACCAGCTCCTACAATCGCCCTGCAGC | |
| | CACTGTCTCTGAGGCCAGAAGCTTGTAGACCTGCTG | |
| | CAGGCGGAGCCGTGCATACAAGAGGACTGGATTTC | |
| | GCCTGCGACTTCTGGGTGCTCGTGGTTGTTGGCGGA | |
| | GTGCTGGCCTGTTACTCTCTGCTGGTCACCGTGGCC | |
| | TTCATCATCTTTTGGGTCCGAAGCAAGCGGAGCCGG | |
| | CTGCTGCACAGCGACTACATGAACATGACCCCTAG | |
| | ACGGCCCGGACCTACCAGAAAGCACTACCAGCCTT | |
| | ACGCTCCTCCTAGAGATTTCGCCGCCTACCGGTCC | |
| | (SEQ ID NO: 111) | |
| SB05917 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD28(H)-CD28(TM)- |
| | CATGCACATCGACGCCACACTGTACACCGAGAGCG | (no-ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTC | |
| | (SEQ ID NO: 112) | |
| SB05918 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD28(H)-CD28(TM)- |
| | CATGCACATCGACGCCACACTGTACACCGAGAGCG | IL15RA(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTCAAGTCCAGACAGACACCTCCTCTGGCCA | |
| | GCGTGGAAATGGAAGCCATGGAAGCTCTGCCTGTG | |
| | ACCTGGGGCACCAGCTCCAGAGATGAGGACCTGGA | |
| | AAACTGCTCCCACCACCTG | |
| | (SEQ ID NO: 113) | |
| SB05919 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | MAG(H)-CD28(TM)- |
| | CATGCACATCGACGCCACACTGTACACCGAGAGCG | CD28(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGATACCCTCCAGTGATCGTGG | |
| | AAATGAACAGCAGCGTGGAAGCCATCGAGGGCTCT | |
| | CATGTGTCTCTGCTGTGTGGCGCCGACAGCAATCCT | |
| | CCTCCTCTGCTGACCTGGATGAGAGATGGCACCGTG | |
| | CTGAGAGAAGCCGTGGCCGAATCTCTGCTGCTGGA | |
| | ACTGGAAGAAGTGACCCCTGCCGAGGATGGCGTGT | |
| | ACGCTTGTCTGGCCGAGAATGCCTACGGCCAGGAC | |
| | AATAGAACCGTGGGCCTGTCCGTGATGTACGCCCCT | |
| | TGGAAGCCTACCGTGAACGGCACAATGGTGGCCGT | |
| | GGAAGGCGAGACAGTGTCCATCCTGTGTAGCACCC | |
| | AGAGCAACCCCGATCCTATCCTGACCATCTTCAAAG | |
| | AGAAGCAGATCCTGAGCACCGTGATCTACGAGAGC | |
| | GAACTGCAGCTCGAACTGCCCGCTGTGTCCCCAGA | |
| | GGATGATGGCGAATATTGGTGCGTGGCAGAGAACC | |
| | AGTACGGCCAGAGAGCCACCGCCTTCAACCTGAGC | |
| | GTGGAATTTGCTCCCGTGCTGCTGCTCGAGAGCCAT | |
| | TGTGCTGCCGCCAGAGATACCGTGCAGTGCCTGTGT | |
| | GTGGTCAAGTCTAACCCCGAGCCTAGCGTGGCCTTT | |
| | GAGCTGCCCAGCAGAAACGTGACCGTGAATGAGAG | |
| | CGAGCGCGAGTTCGTGTACAGCGAGAGATCTGGAC | |
| | TGGTGCTGACCAGCATCCTGACACTGAGAGGACAG | |
| | GCTCAGGCCCCTCCTAGAGTGATCTGCACCGCCAGA | |
| | AATCTGTACGGCGCCAAGAGCCTGGAACTGCCATTT | |
| | CAGGGCGCCCACAGACTCATGTGGGCCAAGATTGG | |
| | ACCTTTCTGGGTGCTCGTGGTTGTTGGCGGAGTGCT | |
| | GGCCTGTTACTCTCTGCTGGTCACCGTGGCCTTCAT | |
| | CATCTTTTGGGTCCGAAGCAAGCGGAGCCGGCTGCT | |
| | GCACAGCGACTACATGAACATGACCCCTAGACGGC | |
| | CCGGACCTACCAGAAAGCACTACCAGCCTTACGCT | |
| | CCTCCTAGAGATTTCGCCGCCTACCGGTCC | |
| | (SEQ ID NO: 114) | |
| SB05920 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD8(H)-OX40(TM)- |
| | CATGCACATCGACGCCACACTGTACACCGAGAGCG | OX40(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAACCACAACTCCTGCTCCTA | |
| | GACCTCCTACACCAGCTCCTACAATCGCCCTGCAGC | |
| | CACTGTCTCTGAGGCCAGAAGCTTGTAGACCTGCTG | |
| | CAGGCGGAGCCGTGCATACAAGAGGACTGGATTTC | |
| | GCCTGCGACGTGGCCGCCATTCTTGGACTGGGACTT | |
| | GTTCTGGGACTGCTGGGACCTCTGGCCATTCTGCTG | |
| | GCTCTGTACCTGCTGAGAAGGGACCAGAGACTGCC | |
| | TCCTGACGCTCACAAACCTCCAGGCGGAGGCAGCT | |
| | TCAGAACCCCTATCCAAGAGGAACAGGCTGACGCC | |
| | CACAGCACCCTGGCCAAGATC | |
| | (SEQ ID NO: 115) | |
| SB05921 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD8(H)-CD8(TM)-4- |
| | CATGCACATCGACGCCACACTGTACACCGAGAGCG | 1BB(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAACCACAACTCCTGCTCCTA | |
| | GACCTCCTACACCAGCTCCTACAATCGCCCTGCAGC | |
| | CACTGTCTCTGAGGCCAGAAGCTTGTAGACCTGCTG | |
| | CAGGCGGAGCCGTGCATACAAGAGGACTGGATTTC | |
| | GCCTGCGACATCTACATCTGGGCCCCTCTGGCTGGA | |
| | ACATGCGGAGTGTTGCTGCTGAGCCTGGTCATCACC | |
| | AAGCGGGGCAGAAAGAAGCTGCTGTACATCTTCAA | |
| | GCAGCCCTTCATGCGGCCCGTGCAGACCACACAAG | |
| | AGGAAGATGGCTGCTCCTGCAGATTCCCCGAGGAA | |
| | GAAGAAGGCGGCTGCGAGCTG | |
| | (SEQ ID NO: 116) | |
| SB05922 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD8(H)-NKp46(TM)- |
| | CATGCACATCGACGCCACACTGTACACCGAGAGCG | NKp46(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAACCACAACTCCTGCTCCTA | |
| | GACCTCCTACACCAGCTCCTACAATCGCCCTGCAGC | |
| | CACTGTCTCTGAGGCCAGAAGCTTGTAGACCTGCTG | |
| | CAGGCGGAGCCGTGCATACAAGAGGACTGGATTTC | |
| | GCCTGCGACATGGGCCTCGCCTTTCTGGTGCTGGTG | |
| | GCCCTTGTGTGGTTCCTGGTGGAAGATTGGCTGAGC | |
| | CGGAAGCGGACAAGAGAGAGAGCCAGCAGAGCCT | |
| | CTACCTGGGAGGGAAGAAGAAGGCTGAACACCCAG | |
| | ACACTC | |
| | (SEQ ID NO: 117) | |
| SB05923 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD8(H)-2B4(TM)- |
| | CATGCACATCGACGCCACACTGTACACCGAGAGCG | 2B4(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAACCACAACTCCTGCTCCTA | |
| | GACCTCCTACACCAGCTCCTACAATCGCCCTGCAGC | |
| | CACTGTCTCTGAGGCCAGAAGCTTGTAGACCTGCTG | |
| | CAGGCGGAGCCGTGCATACAAGAGGACTGGATTTC | |
| | GCCTGCGACTTCCTGGTCATCATCGTGATCCTGAGC | |
| | GCCCTGTTCCTGGGCACCCTGGCCTGTTTTTGCGTG | |
| | TGGCGCCGCAAGCGGAAAGAGAAGCAGTCTGAGAC | |
| | AAGCCCCAAAGAGTTCCTGACCATCTACGAGGACG | |
| | TGAAGGACCTGAAAACCCGGCGGAACCACGAGCAA | |
| | GAGCAGACCTTTCCTGGCGGCGGAAGCACCATCTA | |
| | CAGCATGATCCAGAGCCAGTCTAGCGCCCCTACCA | |
| | GCCAAGAGCCTGCCTACACACTGTACTCCCTGATCC | |
| | AGCCTAGCAGAAAGAGCGGCAGCCGGAAGAGAAA | |
| | TCACAGCCCCAGCTTCAACAGCACGATCTACGAAG | |
| | TGATCGGCAAGAGCCAGCCAAAGGCTCAGAACCCT | |
| | GCCAGGCTGAGCCGGAAAGAGCTGGAAAACTTCGA | |
| | CGTGTACAGC | |
| | (SEQ ID NO: 118) | |
| SB05924 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD28(H)-CD28(TM)- |
| | CATGCACATCGACGCCACACTGTACACCGAGAGCG | OX40(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTCGCTCTGTACCTGCTGAGAAGGGACCAGA | |
| | GACTGCCTCCTGACGCTCACAAACCTCCAGGCGGA | |
| | GGCAGCTTCAGAACCCCTATCCAAGAGGAACAGGC | |
| | TGACGCCCACAGCACCCTGGCCAAGATC | |
| | (SEQ ID NO: 119) | |
| SB05925 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD28(H)-CD28(TM)- |
| | CATGCACATCGACGCCACACTGTACACCGAGAGCG | 4-1BB(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTCAAGCGGGGCAGAAAGAAGCTGCTGTAC | |
| | ATCTTCAAGCAGCCCTTCATGCGGCCCGTGCAGACC | |
| | ACACAAGAGGAAGATGGCTGCTCCTGCAGATTCCC | |
| | CGAGGAAGAAGAAGGCGGCTGCGAGCTG | |
| | (SEQ ID NO: 120) | |
| SB05926 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD28(H)-CD28(TM)- |
| | CATGCACATCGACGCCACACTGTACACCGAGAGCG | NKp46(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTCCGGAAGCGGACAAGAGAGAGAGCCAGC | |
| | AGAGCCTCTACCTGGGAGGGAAGAAGAAGGCTGAA | |
| | CACCCAGACACTC | |
| | (SEQ ID NO: 121) | |
| SB05927 | ATGGACTGGACTTGGATACTCTTTCTGGTCGCTGCC | IgE(SS)-IL-15-linker- |
| | GCCACACGGGTGCACTCTAATTGGGTCAACGTGATC | IL15RA(sushi)-- |
| | AGCGACCTGAAGAAGATCGAGGACCTGATCCAGAG | CD28(H)-CD28(TM)- |
| | CATGCACATCGACGCCACACTGTACACCGAGAGCG | 2B4(ICD) |
| | ACGTGCACCCTAGCTGTAAAGTGACCGCCATGAAG | |
| | TGCTTTCTGCTGGAACTGCAAGTGATCAGCCTGGAA | |
| | AGCGGCGACGCCAGCATCCACGACACCGTGGAAAA | |
| | CCTGATCATCCTGGCCAACAACAGCCTGAGCAGCA | |
| | ACGGCAATGTGACCGAGTCCGGCTGCAAAGAGTGC | |
| | GAGGAACTGGAAGAGAAGAATATCAAAGAGTTCCT | |
| | GCAGAGCTTCGTGCACATCGTGCAGATGTTCATCAA | |
| | CACCAGCAGCGGCGGATCTGGCGGCGGAGGTTCTG | |
| | GCGGTGGAAGCGGAGGCGGAGGATCTCTCCAGATC | |
| | ACATGCCCTCCACCTATGAGCGTGGAACACGCCGA | |
| | CATCTGGGTCAAGAGCTACAGCCTGTACAGCAGAG | |
| | AGCGGTACATCTGCAACAGCGGCTTCAAGAGAAAG | |
| | GCCGGCACAAGCAGCCTGACCGAGTGCGTGCTGAA | |
| | CAAGGCCACAAATGTGGCCCACTGGACCACACCTA | |
| | GCCTGAAGTGCATCAGAGCAGCAGCTATCGAGGTG | |
| | ATGTATCCTCCGCCCTACCTGGATAATGAAAAGAGT | |
| | AATGGGACTATCATTCATGTAAAAGGGAAGCATCT | |
| | TTGTCCTTCTCCCCTTTTCCCCGGTCCGTCTAAACCT | |
| | TTCTGGGTGCTCGTGGTTGTTGGCGGAGTGCTGGCC | |
| | TGTTACTCTCTGCTGGTCACCGTGGCCTTCATCATCT | |
| | TTTGGGTCTGGCGCCGCAAGCGGAAAGAGAAGCAG | |
| | TCTGAGACAAGCCCCAAAGAGTTCCTGACCATCTAC | |
| | GAGGACGTGAAGGACCTGAAAACCCGGCGGAACCA | |
| | CGAGCAAGAGCAGACCTTTCCTGGCGGCGGAAGCA | |
| | CCATCTACAGCATGATCCAGAGCCAGTCTAGCGCCC | |
| | CTACCAGCCAAGAGCCTGCCTACACACTGTACTCCC | |
| | TGATCCAGCCTAGCAGAAAGAGCGGCAGCCGGAAG | |
| | AGAAATCACAGCCCCAGCTTCAACAGCACGATCTA | |
| | CGAAGTGATCGGCAAGAGCCAGCCAAAGGCTCAGA | |
| | ACCCTGCCAGGCTGAGCCGGAAAGAGCTGGAAAAC | |
| | TTCGACGTGTACAGC | |
| | (SEQ ID NO: 122) | |

In certain embodiments, a fusion protein of the present disclosure comprises the amino acid sequence of SEQ ID NO:86. In certain embodiments, a fusion protein of the present disclosure comprises the amino acid sequence of SEQ ID NO:87. In certain embodiments, a fusion protein of the present disclosure comprises the amino acid sequence of SEQ ID NO:88. In certain embodiments, a fusion protein of the present disclosure comprises the amino acid sequence of SEQ ID NO:89. In certain embodiments, a fusion protein of the present disclosure comprises the amino acid sequence of SEQ ID NO:90. In certain embodiments, a fusion protein of the present disclosure comprises the amino acid sequence of SEQ ID NO:91. In certain embodiments, a fusion protein of the present disclosure comprises the amino acid sequence of SEQ ID NO:92. In certain embodiments, a fusion protein of the present disclosure comprises the amino acid sequence of SEQ ID NO:93. In certain embodiments, a fusion protein of the present disclosure comprises the amino acid sequence of SEQ ID NO:94. In certain embodiments, a fusion protein of the present disclosure comprises the amino acid sequence of SEQ ID NO:95. In certain embodiments, a fusion protein of the present disclosure comprises the amino acid sequence of SEQ ID NO:96. In certain embodiments, a fusion protein of the present disclosure comprises the amino acid sequence of SEQ ID NO:97. In certain embodiments, a fusion protein of the present disclosure comprises the amino acid sequence of SEQ ID NO:98. In certain embodiments, a fusion protein of the present disclosure comprises the amino acid sequence of SEQ ID NO:99. In certain embodiments, a fusion protein of the present disclosure comprises the amino acid sequence of SEQ ID NO:100. In certain embodiments, a fusion protein of the present disclosure comprises the amino acid sequence of SEQ ID NO: 101. In certain embodiments, a fusion protein of the present disclosure comprises the amino acid sequence of SEQ ID NO:102. In certain embodiments, a fusion protein of the present disclosure comprises the amino acid sequence of SEQ ID NO:103.

In some embodiments, a cell of the present disclosure expresses a fusion protein that includes an antigen-binding domain that binds a target antigen, a transmembrane domain of the present disclosure, a primary signaling domain, and one or more costimulatory signaling domains.

In some embodiments, polynucleotides encoding any of the fusion protein of the present disclosure may comprise any of the nucleic acid sequences listed in Table 6, or a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 6 above.

### Chimeric Antigen Receptors (CARs)

Certain aspects of the present disclosure relate to chimeric receptors for use in combination with a fusion protein described herein. In some embodiments, the chimeric receptor is a chimeric antigen receptor (CAR).

In general, CARs are chimeric proteins that include an antigen-binding domain and polypeptide molecules that are heterologous to the antigen-binding domain, such as peptides heterologous to an antibody that an antigen-binding domain may be derived from. Polypeptide molecules that are heterologous to the antigen-binding domain can include, but are not limited to, a transmembrane domain, one or more intracellular signaling domains, a hinge domain, a spacer region, one or more peptide linkers, or combinations thereof.

In some embodiments, CARs are engineered receptors that graft or confer a specificity of interest onto an immune effector cell. In certain embodiments, CARs can be used to graft the specificity of an antibody onto an immunoresponsive cell, such as a T cell or an NK cell. In some embodiments, CARs of the present disclosure comprise an extracellular antigen-binding domain (e.g., an scFv) fused to a transmembrane domain, fused to one or more intracellular signaling domains. In some embodiments, CARs of the present disclosure comprise an extracellular antigen-binding domain (e.g., a receptor ligand or a receptor-binding domain) fused to a transmembrane domain, fused to one or more intracellular signaling domains. In some embodiments, CARs of the present disclosure comprise an extracellular antigen-binding domain (e.g., a receptor or a ligand-binding domain) fused to a transmembrane domain, fused to one or more intracellular signaling domains.

In some embodiments, the chimeric antigen receptor is an activating chimeric antigen receptor (aCAR and also generally referred to as CAR unless otherwise specified). In some embodiments, binding of the chimeric antigen receptor to its cognate ligand is sufficient to induce activation of the immunoresponsive cell. In some embodiments, binding of the chimeric antigen receptor to its cognate ligand is sufficient to induce stimulation of the immunoresponsive cell. In some embodiments, activation of an immunoresponsive cell results in killing of target cells. In some embodiments, activation of an immunoresponsive cell results in cytokine expression and/or secretion by the immunoresponsive cell. In some embodiments, stimulation of an immunoresponsive cell results in cytokine expression and/or secretion by the immunoresponsive cell. In some embodiments, stimulation of an immunoresponsive cell induces differentiation of the immunoresponsive cell. In some embodiments, stimulation of an immunoresponsive cell induces proliferation of the immunoresponsive cell. In some embodiments, activation and/or stimulation of the immunoresponsive cell can be combinations of the above responses.

A CAR of the present disclosure may be a first, second, or third generation CAR. “First generation” CARs comprise a single intracellular signaling domain, generally derived from a T cell receptor chain. “First generation” CARs generally have the intracellular signaling domain from the CD3-zeta (CD3ζ) chain, which is the primary transmitter of signals from endogenous TCRs. “First generation” CARs can provide de novo antigen recognition and cause activation of both CD4+ and CD8+ T cells through their CD35 chain signaling domain in a single fusion molecule, independent of HLA-mediated antigen presentation. “Second generation” CARs add a second intracellular signaling domain from one of various co-stimulatory molecules (e.g., CD28, 4-1BB, ICOS, OX40) to the cytoplasmic tail of the CAR to provide additional signals to the T cell. “Second generation” CARs provide both co-stimulation (e.g., CD28 or 4-1BB) and activation (CD3ζ). Preclinical studies have indicated that “Second Generation” CARs can improve the anti-tumor activity of immunoresponsive cell, such as a T cell. “Third generation” CARs have multiple intracellular co-stimulation signaling domains (e.g., CD28 and 4-1BB) and an intracellular activation signaling domain (CD3ζ).

In some embodiments, the chimeric antigen receptor is a chimeric inhibitory receptor (iCAR). In some embodiments, chimeric inhibitory receptors bind antigens that are expressed on a non-tumor cell derived from a tissue selected from brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor may be used, for example, with one or more activating chimeric receptors (e.g., activating chimeric TCRs or CARs) expressed on a cell of the present disclosure (e.g., an immunoresponsive cell) as NOT-logic gates to control, modulate, or otherwise inhibit one or more activities of the one or more activating chimeric receptors. For instance, if a healthy cell expresses both an antigen that is recognized by a tumor-targeting chimeric receptor and an antigen that is recognized by an inhibitory chimeric receptor, an immunoresponsive cell expressing the tumor antigen may bind to the healthy cell. In such a case, the inhibitory chimeric antigen will also bind its cognate ligand on the healthy cell and the inhibitory function of the inhibitory chimeric receptor will reduce, decrease, prevent, or inhibit the activation of the immunoresponsive cell via the tumor-targeting chimeric receptor (“NOT-logic gating”). In some embodiments, a chimeric inhibitory receptor of the present disclosure may inhibit one or more activities of a cell of the present disclosure (e.g., an immunoresponsive cell). In some embodiments, an immunoresponsive cell may comprise one or more tumor-targeting chimeric receptors and one or more inhibitory chimeric receptors that targets an antigen that is not expressed, or generally considered to be expressed, on the tumor. Combinations of tumor-targeting chimeric receptors and inhibitory chimeric receptors in the same immunoresponsive cell may be used to reduce on-target off-tumor toxicity.

In some embodiments, the extracellular antigen-binding domain of a CAR of the present disclosure binds to one or more antigens with a dissociation constant (K_{d}) of about 2×10⁻⁷ M or less, about 1×10⁻⁷M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ M or less, about 2×10⁻⁹ M or less, or about 1×10⁻⁹ M or less. In some embodiments, the K_{d} ranges from about is about 2×10⁻⁷ M to about 1×10⁻⁹ M.

Binding of the extracellular antigen-binding domain of a CAR of the present disclosure can be determined by, for example, an enzyme-linked immunosorbent assay (ELISA), a radioimmunoassay (RIA), FACS analysis, a bioassay (e.g., growth inhibition), bio-layer interferometry (e.g., Octet/FORTEBIO®), surface plasmon resonance (SPR) technology (e.g., Biacore®), or a Western Blot assay. Each of these assays generally detect the presence of protein-antibody complexes of particular interest by employing a labeled reagent (e.g., an antibody or scFv) specific for the complex of interest. For example, the scFv can be radioactively labeled and used in an RIA assay. The radioactive isotope can be detected by such means as the use of a y counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the extracellular antigen-binding domain of the CAR is labeled with a secondary antibody specific for the extracellular antigen-binding domain and wherein the secondary antibody is labeled (e.g., radioactively or with a fluorescent marker).

In some embodiments, CARs of the present disclosure comprise an extracellular antigen-binding domain that binds to a target antigen, a transmembrane domain, and one or more intracellular signaling domains. In some embodiments, the extracellular antigen-binding domain comprises an scFv. In some embodiments, the extracellular antigen-binding domain comprises a Fab fragment, which may be crosslinked. In certain embodiments, the extracellular binding domain is a F(ab)₂ fragment

### Extracellular Antigen-Binding Domain

The extracellular antigen-binding domain of a CAR of the present disclosure specifically binds to a target antigen (e.g., a target antigen protein, a target antigen-derived antigen, or a target antigen-derived epitope). In certain embodiments, the extracellular antigen-binding domain binds to a target antigen expressed on an epithelial cell. In certain embodiments, the extracellular antigen-binding domain binds to a target antigen expressed on cells generally considered to be healthy, such as healthy epithelial cells. In some embodiments, a target antigen is a human target antigen.

Antigen-binding domains of the present disclosure can include any domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (V_{H}H) of camelid derived nanobody, and to an alternative scaffold known in the art to function as antigen-binding domain, such as a recombinant fibronectin domain, a T cell receptor (TCR), a recombinant TCR with enhanced affinity, or a fragment thereof, e.g., single chain TCR, and the like. In some instances, it is beneficial for the antigen-binding domain to be derived from the same species in which the CAR will ultimately be used in.

In some embodiments, the extracellular antigen-binding domain comprises an antibody. In certain embodiments, the antibody is a human antibody. In certain embodiments, the antibody is a chimeric antibody. In some embodiments, the extracellular antigen-binding domain comprises an antigen-binding fragment of an antibody.

In some embodiments, the extracellular antigen-binding domain comprises a F(ab) fragment. In certain embodiments, the extracellular antigen-binding domain comprises a F(ab′) fragment.

In some embodiments, the extracellular antigen-binding domain comprises an scFv. In some embodiments, the extracellular antigen-binding domain comprises two single chain variable fragments (scFvs). In some embodiments, each of the two scFvs binds to a distinct epitope on the same antigen. In some embodiments, the extracellular antigen-binding domain comprises a first scFv and a second scFv. In some embodiments, the first scFv and the second scFv bind distinct epitopes on the same antigen. In certain embodiments, the scFv is a mammalian scFv. In certain embodiments, the scFv is a chimeric scFv. In certain embodiments, the scFv comprises a heavy chain variable domain (VH) and a light chain variable domain (VL). In certain embodiments, the VH and VL are separated by a peptide linker. In certain embodiments, the peptide linker comprises any of the amino acid sequences shown in Table 1. In certain embodiments, the scFv comprises the structure VH-L-VL or VL-L-VH, wherein VH is the heavy chain variable domain, L is the peptide linker, and VL is the light chain variable domain. In some embodiments, each of the one or more scFvs comprises the structure VH-L-VL or VL-L-VH, wherein VH is the heavy chain variable domain, L is the peptide linker, and VL is the light chain variable domain. When there are two or more scFv linked together, each scFv can be linked to the next scFv with a peptide linked. In some embodiments, each of the one or more scFvs is separated by a peptide linker.

In some embodiments, the present disclosure provides a first CAR and a second CAR. The antigen binding domain of the first CAR and the antigen binding domain of the second CAR can be an appropriate antigen biding domain described herein or known in the art. For example, the first or second antigen binding domain can be one or more antibodies, antigen-binding fragments of an antibody, F(ab) fragments, F(ab′) fragments, single chain variable fragments (scFvs), or single-domain antibodies (sdAbs). In some embodiments, the antigen-binding domain of the first CAR and/or the second CAR comprises two single chain variable fragments (scFvs). In some embodiments, each of the two scFvs binds to a distinct epitope on the same antigen. In some embodiments, the antigen binding domain of the first CAR can be specific for a target antigen and the antigen binding domain of the second CAR can be specific for a second distinct antigen, such as a tumor-associated antigen (e.g., an antigen expressed on a tumor cell).

In some embodiments, the tumor-associated antigen is chosen from one or more of: CD19; CD123; CD22; CD30; CD70; CD171; CS-1 (also referred to as CD2 subset 1, CRACC, SLAMF7, CD319, and 19A24); C-type lectin-like molecule-1 (CLL-1 or CLEC12A); CD33; epidermal growth factor receptor variant III (EGFRvIII); ganglioside G2 (GD2); ganglioside GD3 (aNcu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); CEACAM1, CEACAM5, CEACAM6, Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC-1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNcu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEMI/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGEI); angiopoietin-binding cell surface receptor 2 (Tic 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoman antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYPIB 1); CCCTC-Binding Factor (Zinc Finger Protein)-Like (BORIS or Brother of the Regulator of Imprinted Sites), Squamous Cell Carcinoman antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the extracellular antigen-binding domain comprises a single-domain antibody (sdAb). In certain embodiments, the sdAb is a humanized sdAb. In certain embodiments, the sdAb is a chimeric sdAb.

In some embodiments, a CAR of the present disclosure may comprise two or more antigen-binding domains, three or more antigen-binding domains, four or more antigen-binding domains, five or more antigen-binding domains, six or more antigen-binding domains, seven or more antigen-binding domains, eight or more antigen-binding domains, nine or more antigen-binding domains, or ten or more antigen-binding domains. In some embodiments, each of the two or more antigen-binding domains binds the same antigen. In some embodiments, each of the two or more antigen-binding domains binds a different epitope of the same antigen. In some embodiments, each of the two or more antigen-binding domains binds a different antigen.

In some embodiments, the CAR comprises two antigen-binding domains. In some embodiments, the two antigen-binding domains are attached to one another via a flexible linker. In some embodiments, each of the two-antigen-binding domains may be independently selected from an antibody, an antigen-binding fragment of an antibody, an scFv, a sdAb, a recombinant fibronectin domain, a T cell receptor (TCR), a recombinant TCR with enhanced affinity, and a single chain TCR. In some embodiments, the CAR comprising two antigen-binding domains is a bispecific CAR or a tandem CAR (tanCAR).

In certain embodiments, the bispecific CAR or tanCAR comprises an antigen-binding domain comprising a bispecific antibody or antibody fragment (e.g., scFv). In some embodiments, within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁ VH₁-VH₂-VL₂. In some embodiments, a linker is disposed between the two antibodies or antibody fragments (e.g., scFvs), for example, between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser) n linker, wherein n is 1, 2, 3, 4, 5, or 6. In general, the linker between the two scFvs should be long enough to avoid mispairing between the domains of the two scFvs. In some embodiments, a linker is disposed between the VL and VH of the first scFv. In some embodiments, a linker is disposed between the VL and VH of the second scFv. In constructs that have multiple linkers, any two or more of the linkers may be the same or different. Accordingly, in some embodiments, a bispecific CAR or tanCAR comprises VLs, VHs, and may further comprise one or more linkers in an arrangement as described herein.

### Transmembrane Domain

In some embodiments, the transmembrane domain of a CAR of the present disclosure comprises a hydrophobic alpha helix that spans at least a portion of a cell membrane. It has been shown that different transmembrane domains can result in different receptor stability. After antigen recognition, receptors cluster and a signal is transmitted to the cell. In some embodiments, the transmembrane domain of a CAR of the present disclosure can comprise the transmembrane domain of a CD25 polypeptide, a CD7 polypeptide, a LAX polypeptide, a LAT polypeptide, a TIM3 polypeptide, a KIR3DS1 polypeptide, a KIR3DL1 polypeptide, an NKG2D polypeptide, an NKG2A polypeptide, a TIGIT polypeptide, a CD8 polypeptide, a CD28 polypeptide, a CD3-zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a LIR-1 (LILRB1) polypeptide, or can be a synthetic peptide, or any combination thereof.

In some embodiments, the transmembrane domain is derived from a CD8 polypeptide. Any suitable CD8 polypeptide may be used. Exemplary CD8 polypeptides include, without limitation, NCBI Reference Nos. NP_001139345 and AAA92533.1. Examples of CD8 transmembrane domains include IYIWAPLAGTCGVLLLSLVIT (SEQ ID NO: 36), IYIWAPLAGTCGVLLLSLVITLYCNHR (SEQ ID NO:37), and IYIWAPLAGTCGVLLLSLVITLYCNHRN (SEQ ID NO:38). In some embodiments, the transmembrane domain comprises the sequence IYIWAPLAGTCGVLLLSLVIT (SEQ ID NO: 36). In some embodiments, the transmembrane domain comprises the sequence IYIWAPLAGTCGVLLLSLVITLYCNHR (SEQ ID NO:37). In some embodiments, the transmembrane domain comprises the sequence IYIWAPLAGTCGVLLLSLVITLYCNHRN (SEQ ID NO:38).

In some embodiments, the transmembrane domain is derived from a CD25 polypeptide. Any suitable CD25 polypeptide may be used. Exemplary CD25 polypeptides include, without limitation, NCBI Reference Nos. NP_001295171.1, NP_001295172.land NP_000408.1. In some embodiments, the transmembrane domain is derived from a CD28 polypeptide. Any suitable CD28 polypeptide may be used. Exemplary CD28 polypeptides include, without limitation, NCBI Reference Nos. NP_006130.1 and NP_031668.3. In some embodiments, the transmembrane domain is derived from a CD7 polypeptide. Any suitable CD7 polypeptide may be used. Exemplary CD7 polypeptides include, without limitation, NCBI Reference Nos. NP_006128.1. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1.

In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide.

Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. In some embodiments, the transmembrane domain is derived from a LIR-1 (LILRB1) polypeptide. Any suitable LIR-1 (LILRB1) polypeptide may be used. Exemplary LIR-1 (LILRB1) polypeptides include, without limitation, NCBI Reference Nos. NP_001075106.2 and NP_001075107.2. In some embodiments, the transmembrane domain is derived from a LAX polypeptide. Any suitable LAX polypeptide may be used. Exemplary LAX polypeptides include, without limitation, NCBI Reference Nos. NP_001129662.1, NP_001269807.1 and NP_060243.2. In some embodiments, the transmembrane domain is derived from a LAT polypeptide. Any suitable LAT polypeptide may be used. Exemplary LAT polypeptides include, without limitation, NCBI Reference Nos. NP_001014987.1, NP_001014988.1, NP_001014989.2 and NP_055202.1. In some embodiments, the transmembrane domain is derived from a KIR3DS1 polypeptide. Any suitable KIR3DS1 polypeptide may be used. Exemplary KIR3DS1 polypeptides include, without limitation, NCBI Reference Nos. NP_001077008.1, NP_001269099.1, and NP_001269100.1. In some embodiments, the transmembrane domain is derived from a KIR3DL1 polypeptide. Any suitable KIR3DL1 polypeptide may be used. Exemplary KIR3DL1 polypeptides include, without limitation, NCBI Reference Nos. NP_001309097.1 and NP_037421.2. In some embodiments, the transmembrane domain is derived from an NKG2D polypeptide. Any suitable NKG2D polypeptide may be used. Exemplary NKG2D polypeptides include, without limitation, NCBI Reference No. NP_031386.2. In some embodiments, the transmembrane domain is derived from an NKG2A polypeptide. Any suitable NKG2A polypeptide may be used. Exemplary NKG2A polypeptides include, without limitation, NCBI Reference No. NP_001162059.1. In some embodiments, the transmembrane domain is derived from a TIGIT polypeptide. Any suitable TIGIT polypeptide may be used. Exemplary TIGIT polypeptides include, without limitation, NCBI Reference No. NP_776160.2. In some embodiments, the transmembrane domain is derived from an IL-15Rα polypeptide. Any suitable IL-15Rα polypeptide may be used. Exemplary IL-15Rα polypeptides include, without limitation, NCBI Reference Nos. NP_001243694.1, NP_001230468.1, and NP_001338026.1. In some embodiments, the transmembrane domain is derived from a CD8A polypeptide. Any suitable CD8A polypeptide may be used. Exemplary CD8A polypeptides include, without limitation, NCBI Reference Nos. NP_001139345.1 and NP_001759.3. In some embodiments, the transmembrane domain is derived from an NKp46 polypeptide. Any suitable NKp46 polypeptide may be used. Exemplary NKp4 polypeptides include, without limitation, NCBI Reference Nos. NP_004820.2, NP_001138929.2 and NP_001138930.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2, or fragments thereof. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NG2C.

### Spacer Region

In some embodiments, a CAR of the present disclosure can also comprise a spacer region that links the extracellular antigen-binding domain to the transmembrane domain. The spacer region may be flexible enough to allow the antigen-binding domain to orient in different directions to facilitate antigen recognition. In some embodiments, the spacer region may be a hinge from a human protein. For example, the hinge may be a human Ig (immunoglobulin) hinge, including without limitation an IgG4 hinge, an IgG2 hinge, a CD8a hinge, or an IgD hinge. In some embodiments, the spacer region may comprise an IgG4 hinge, an IgG2 hinge, an IgD hinge, a CD28 hinge, a KIR2DS2 hinge, an LNGFR hinge, or a PDGFR-beta extracellular linker. In some embodiments, the spacer region is localized between the antigen-binding domain and the transmembrane domain. In some embodiments, a spacer region may comprise any of the amino acid sequences listed in Table 2, or an amino acid sequence that is at least 90%, at least 91%, at least 92%, at least 93%, at least 94%, at least 95%, at least 96%, at least 97%, at least 98%, or at least 99% identical to any of the amino acid sequences listed in Table 2 above.

In some embodiments, the spacer region comprises the sequence shown in SEQ ID NO:39. In some embodiments, the spacer region comprises the sequence shown in SEQ ID NO:40. In some embodiments, the spacer region comprises the sequence shown in SEQ ID NO:41. In some embodiments, the spacer region comprises the sequence shown in SEQ ID NO:42. In some embodiments, the spacer region comprises the sequence shown in SEQ ID NO:43. In some embodiments, the spacer region comprises the sequence shown in SEQ ID NO:44. In some embodiments, the spacer region comprises the sequence shown in SEQ ID NO:45. In some embodiments, the spacer region comprises the sequence shown in SEQ ID NO:46. In some embodiments, the spacer region comprises the sequence shown in SEQ ID NO:47. In some embodiments, the spacer region comprises the sequence shown in SEQ ID NO:48.

In some embodiments, the spacer region comprises the sequence TTTPAPRPPTPAPTIALQPLSLRPEACRPAAGGAVHTRGLDFACD (SEQ ID NO:49). In some embodiments, the spacer region comprises the sequence ALSNSIMYFSHFVPVFLPAKPTTTPAPRPPTPAPTIASQPLSLRPEACRPAAGGAVHTR GLDFACD (SEQ ID NO:50). In some embodiments, the spacer region comprises the sequence

(SEQ ID NO: 51)
FVPVFLPAKPTTTPAPRPPTPAPTIALQPLSLRPEACRPAAGGAVHTRG
LDFACD.

In some embodiments, polynucleotides encoding any of the spacer regions of the present disclosure may comprise any of the nucleic acid sequences listed in Table 3, or a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 3 above.

In some embodiments, a CAR of the present disclosure may further include a short oligopeptide or polypeptide linker that is between 2 amino acid residues and 10 amino acid residues in length, and that may form the linkage between the transmembrane domain and the cytoplasmic region of the CAR. A non-limiting example of a suitable linker is a glycine-serine doublet. In some embodiments, the linker comprises the ammo acid sequence of GGCKJSGGCKJS (SEQ ID NO:62).

### Intracellular Signaling Domains

In some embodiments, a CAR of the present disclosure comprises one or more cytoplasmic domains or regions. The cytoplasmic domain or region of the CAR may include an intracellular signaling domain.

Examples of suitable intracellular signaling domains that may be used in CARs of the present disclosure include, without limitation, cytoplasmic sequences of the T cell receptor (TCR) and co-receptors that act in concert to modulate signal transduction following antigen receptor engagement, as well as any derivative or variant of these sequences and any recombinant sequence that has the same functional capability.

Without wishing to be bound by theory, it is believed that signals generated through the TCR alone are insufficient for full activation of the T cell and that a secondary and/or co-stimulatory signal is thus also typically utilized for full activation. Accordingly, T cell activation may be mediated by two distinct classes of cytoplasmic signaling sequences, those that initiate antigen-dependent primary activation through the TCR (primary intracellular signaling domains) and those that act in an antigen-independent manner to provide a secondary or co-stimulatory signal (secondary cytoplasmic domain, e.g., a co-stimulatory domain). In addition, T cell signaling and function (e.g., an activating signaling cascade) can be negatively regulated by inhibitory receptors present in a T cell through intracellular inhibitory co-signaling domains.

In some embodiments, the intracellular signaling domain of a CAR of the present disclosure can include an inhibitory intracellular signaling domains. In some embodiments, the inhibitory intracellular signaling domain includes one or more intracellular inhibitory co-signaling domains. In some embodiments, the one or more intracellular inhibitory co-signaling domains are linked to other domains (e.g., a transmembrane domain) through a peptide linker (e.g., see Table 1) or a spacer or hinge sequence (e.g., see Table 2). In some embodiments, when two or more intracellular inhibitory co-signaling domains are present, the two or more intracellular inhibitory co-signaling domains can be linked through a peptide linker (e.g., see Table 1) or a spacer or hinge sequence (e.g., see Table 2). In some embodiments, the intracellular inhibitory co-signaling domain is an inhibitory domain. In some embodiments, the one or more intracellular inhibitory co-signaling domains of a chimeric protein comprises one or more ITIM-containing protein, or fragment(s) thereof. ITIMs are conserved amino acid sequences found in cytoplasmic tails of many inhibitory immune receptors. In some embodiments, the one or more ITIM-containing protein, or fragments thereof, is selected from PD-1, CTLA4, TIGIT, BTLA, and LAIR1. In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAG3, HAVR, GITR, and PD-L1. The inhibitory intracellular signaling domain can further include an enzymatic inhibitory domain. In some embodiments, the enzymatic inhibitory domain comprises an enzyme catalytic domain. In some embodiments, the enzyme catalytic domain is derived from an enzyme including, but not limited to, CSK, SHP-1, PTEN, CD45, CD148, PTP-MEG1, PTP-PEST, c-CBL, CBL-b, PTPN22, LAR, PTPH1, SHIP-1, or RasGAP. Examples of enzymatic regulation of signaling is described in more detail in Pavel Otáhal et al. (Biochim Biophys Acta. 2011 February; 1813 (2): 367-76), Kosugi A., et al. (Involvement of SHP-1 tyrosine phosphatase in TCR-mediated signaling pathways in lipid rafts, Immunity, 2001 June; 14 (6): 669-80), and Stanford, et al. (Regulation of TCR signaling by tyrosine phosphatases: from immune homeostasis to autoimmunity, Immunology, 2012 September; 137 (1): 1-19), each of which is incorporated herein by reference for all purposes.

In some embodiments, the intracellular signaling domain of a CAR of the present disclosure can comprise a primary signaling domain regulates primary activation of the TCR complex either in a stimulatory way or in an inhibitory way. Primary intracellular signaling domains that act in a stimulatory manner may contain signaling motifs which are known as immunoreceptor tyrosine-based activation motifs (ITAMs). Examples of suitable ITAM-containing primary intracellular signaling domains that that may be used in the CARs of the present disclosure include, without limitation, those of CD3-zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d.

In some embodiments, a CAR of the present disclosure comprises an intracellular signaling domain, e.g., a primary signaling domain of CD3-zeta polypeptide. A CD3-zeta polypeptide of the present disclosure may have an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_932170 or NP_001106864.2, or fragments thereof. In some embodiments, the CD3-zeta polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_932170 or NP_001106864.2 that is at least 20, at least 30, at least 40, at least 50, at least 60, at least 70, at least 80, at least 90, at least 100, at least 110, at least 120, at least 130, at least 140, at least 150, or at least 160, at least 170, or at least 180 amino acids in length.

In other embodiments, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In one embodiment, a primary signaling domain comprises one, two, three, four or more ITAM motifs.

In some embodiments, the intracellular signaling domain of a CAR of the present disclosure can comprise the CD3-zeta signaling domain by itself, or it can be combined with any other desired intracellular signaling domain(s) useful in the context of a CAR of the present disclosure. For example, the intracellular signaling domain of the CAR can comprise a CD3-zeta chain portion and a costimulatory signaling domain. The costimulatory signaling domain may refer to a portion of the CAR comprising the intracellular domain of a costimulatory molecule. A costimulatory molecule of the present disclosure is a cell surface molecule other than an antigen receptor or its ligands that may be utilized for an efficient response of lymphocytes to an antigen. Examples of suitable costimulatory molecules include, without limitation, CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and the like.

In some embodiments, the intracellular signaling sequences within the cytoplasmic portion of a CAR of the present disclosure may be linked to each other in a random or specified order. In some embodiments, a short oligopeptide or polypeptide linker, for example, between 2 amino acids and 10 amino acids (e.g., 2 amino acids, 3 amino acids, 4 amino acids, 5 amino acids, 6 amino acids, 7 amino acids, 8 amino acids, 9 amino acids, or 10 amino acids) in length may form the linkage between intracellular signaling sequences. In one embodiment, a glycine-serine doublet can be used as a suitable linker. In one embodiment, a single ammo acid, e.g., an alanine or a glycine, can be used as a suitable linker.

In some embodiments, the intracellular signaling domain comprises two or more costimulatory signaling domains, e.g., two costimulatory signaling domains, three costimulatory signaling domains, four costimulatory signaling domains, five costimulatory signaling domains, six costimulatory signaling domains, seven costimulatory signaling domains, eight costimulatory signaling domains, nine costimulatory signaling domains, 10 costimulatory signaling domains, or more costimulatory signaling domains. In one embodiment, the intracellular signaling domain comprises two costimulatory signaling domains. In some embodiments, the two or more costimulatory signaling domains are separated by a linker of the present disclosure (e.g., any of the linkers described in Table 1). In one embodiment, the linker is a glycine residue. In another embodiment, the linker is an alanine residue.

In some embodiments, a cell of the present disclosure expresses a CAR that includes an antigen-binding domain that binds a target antigen, a transmembrane domain of the present disclosure, a primary signaling domain, and one or more costimulatory signaling domains.

In some embodiments, a cell of the present disclosure expresses an iCAR that includes an antigen-binding domain that binds a target antigen, a transmembrane domain of the present disclosure, and one or more intracellular inhibitory co-signaling domains. In some embodiments, a cell of the present disclosure expresses (1) a CAR that includes an antigen-binding domain that binds a target antigen, a transmembrane domain of the present disclosure, a primary signaling domain, and one or more costimulatory signaling domains.

### Natural Killer Cell Receptor (NKR) CARs

In some embodiments, a CAR of the present disclosure comprises one or more components of a natural killer cell receptor (NKR), thereby forming an NKR-CAR. The NKR component may be a transmembrane domain, a hinge domain, or a cytoplasmic domain from any suitable natural killer cell receptor, including without limitation, a killer cell immunoglobulin-like receptor (KIR), such as KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2 DPI, and KIRS DPI; a natural cytotoxicity receptor (NCR), such as NKp30, NKp44, NKp46; a signaling lymphocyte activation molecule (SLAM) family of immune cell receptor, such as CD48, CD229, 2B4, CD84, NTB-A, CRACC, BLAME, and CD2F-10; an Fc receptor (FcR), such as CD16, and CD64; and an Ly49 receptor, such as LY49A and LY49C. In some embodiments, the NKR-CAR may interact with an adaptor molecule or intracellular signaling domain, such as DAP12. Exemplary configurations and sequences of CARs comprising NKR components are described in International Patent Publication WO2014/145252, published Sep. 18, 2014.

### Additional Chimeric Receptor Targets

Certain aspects of the present disclosure relate to chimeric receptors and polynucleotides that encode such chimeric receptors that bind to an antigen of interest in addition to a target antigen. Certain aspects of the present disclosure relate to chimeric receptors and cells, such as immunoresponsive cells, that have been genetically modified to express one or more of such chimeric receptors that bind to an antigen of interest in addition to a target antigen, and to methods of using such receptors and cells to treat and/or prevent malignancies, such as solid tumors, and other pathologies where an antigen-specific immune response is desired. Malignant cells have developed a series of mechanisms to protect themselves from immune recognition and elimination. The present disclosure provides immunogenicity within the tumor microenvironment for treating such malignant cells.

In some embodiments, a first chimeric receptor includes an antigen-binding domain that binds a target antigen, and a second chimeric receptor includes an additional antigen-binding domain that binds a second antigen, such as a tumor-associated antigen. In some embodiments, a cell can express a first chimeric receptor specific for a target antigen and a second chimeric receptor specific for a second antigen, such as a tumor-associated antigen. In some embodiments, a cell can express a first inhibitory chimeric receptor specific for a target antigen and a second chimeric receptor specific for a second antigen, such as a tumor-associated antigen. For example, a cell (e.g., an immunoresponsive cell) can be engineered to co-expresses or capable of co-expressing an iCAR that includes an antigen-binding domain that binds a target antigen and an aCAR that targets a tumor-associated antigen. Suitable antibodies that bind to an antigen in addition to a target antigen include any antibody, whether natural or synthetic, full length or a fragment thereof, monoclonal or polyclonal, that binds sufficiently strongly and specifically to a second antigen, such a tumor-associated antigen. In some embodiments, commercially available antibodies may be used for binding to a second antigen, such a tumor-associated antigen. The CDRs of the commercially available antibodies are readily accessible by one skilled in the art using conventional sequencing technology. Further, one skilled in the art is able to construct polynucleotides encoding scFvs and chimeric receptors (e.g., CARs and TCRs) based on the CDRs of such commercially available antibodies.

### T Cell Receptor (TCR)

Certain aspects of the present disclosure relate to chimeric receptors that specifically bind to a second antigen, such a tumor-associated antigen and the chimeric receptor for the second antigen is an engineered T cell receptor (TCR). TCRs of the present disclosure are disulfide-linked heterodimeric proteins containing two variable chains expressed as part of a complex with the invariant CD3 chain molecules. TCRs are found on the surface of T cells, and are responsible for recognizing antigens as peptides bound to major histocompatibility complex (MHC) molecules. In certain embodiments, a TCR of the present disclosure comprises an alpha chain encoded by TRA, and a beta chain encoded by TRB. In certain embodiments, a TCR comprises a gamma chain and a delta chain (encoded by TRG and TRD, respectively).

Each chain of a TCR is composed of two extracellular domains: a variable (V) region and a constant (C) region. The constant region is proximal to the cell membrane, followed by a transmembrane region and a short cytoplasmic tail. The variable region binds to the peptide/MHC complex. Each of the variable regions has three complementarity determining regions (CDRs).

In certain embodiments, a TCR can form a receptor complex with three dimeric signaling modules CD38/8, CD3y/s, and CD2474/3 or CD247Y/n. When a TCR complex engages with its antigen and MHC (peptide/MHC), the T cell expressing the TCR complex is activated.

In some embodiments, a TCR of the present disclosure is a recombinant TCR. In certain embodiments, the TCR is a non-naturally occurring TCR. In certain embodiments, the TCR differs from a naturally occurring TCR by at least one amino acid residue. In some embodiments, the TCR differs from a naturally occurring TCR by at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, at least 13 amino acid residues, at least 14 amino acid residues, at least 15 amino acid residues, at least 20 amino acid residues, at least 25 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, or more amino acid residues. In certain embodiments, the TCR is modified from a naturally occurring TCR by at least one amino acid residue. In some embodiments, the TCR is modified from a naturally occurring TCR by at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, at least 13 amino acid residues, at least 14 amino acid residues, at least 15 amino acid residues, at least 20 amino acid residues, at least 25 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, or more amino acid residues.

### Chimeric TCRs

In some embodiments, a TCR of the present disclosure comprises one or more antigen-binding domains that may be grafted to one or more constant domain of a TCR chain, for example a TCR alpha chain or TCR beta chain, to create a chimeric TCR that binds specifically to a second antigen of interest, such a tumor-associated antigen. Without wishing to be bound by theory, it is believed that chimeric TCRs may signal through the TCR complex upon antigen binding. For example, an antibody or antibody fragment (e.g., scFv) can be grafted to the constant domain, e.g., at least a portion of the extracellular constant domain, the transmembrane domain and the cytoplasmic domain, of a TCR chain, such as the TCR alpha chain and/or the TCR beta chain. As another example, the CDRs of an antibody or antibody fragment may be grafted into a TCR alpha chain and/or beta chain to create a chimeric TCR that binds specifically to a second antigen, such a tumor-associated antigen. Such chimeric TCRs may be produced by methods known in the art (e.g., Willemsen R A et al., Gene Therapy 2000; 7:1369-1377; Zhang T et al., Cancer Gene Ther 2004 11:487-496; and Aggen et al., Gene Ther. 2012 April; 19 (4): 365-74).

### Immunoresponsive Cells

Certain aspects of the present disclosure relate to a cell, such as an immunoresponsive cell, that has been genetically engineered to comprise one or more chimeric receptors of the present disclosure or one or more polynucleotides encoding such chimeric receptors, and to methods of using such cells for treating solid tumors.

In some embodiments, the cell is a mammalian cell. In some embodiments, the mammalian cell is a primary cell. In some embodiments, the mammalian cell is a cell line. In some embodiments, the mammalian cell a bone marrow cell, a blood cell, a skin cell, bone cell, a muscle cell, a neuronal cell, a fat cell, a liver cell, or a heart cell. In some embodiments, the cell is a stem cell. Exemplary stem cells include, without limitation embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is a cell that is derived or differentiated from a stem cell of the present disclosure. In some embodiments, the cell is an immune cell. Immune cells of the present disclosure may be isolated or differentiated from a stem cell of the present disclosure (e.g., from an ESC or iPSC). Exemplary immune cells include, without limitation, T cells (e.g., helper T cells, cytotoxic T cells, memory T cells, regulatory T cells, natural killer T cells, alpha beta T cells, and gamma delta T cells), B cells, natural killer (NK) cells, dendritic cells, myeloid cells, macrophages, and monocytes. In some embodiments, the cell is a neuronal cell. Neuronal cells of the present disclosure may be isolated or differentiated from a stem cell of the present disclosure (e.g., from an ESC or iPSC). Exemplary neuronal cells include, without limitation, neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the cell is an immunoresponsive cell. Immunoresponsive cells of the present disclosure may be isolated or differentiated from a stem cell of the present disclosure (e.g., from an ESC or iPSC). Exemplary immunoresponsive cells of the present disclosure include, without limitation, cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Examples of immunoresponsive cells of the lymphoid lineage include, without limitation, T cells, Natural Killer (NK) cells, embryonic stem cells, pluripotent stem cells, and induced pluripotent stem cells (e.g., those from which lymphoid cells may be derived or differentiated). T cells can be lymphocytes that mature in the thymus and are chiefly responsible for cell-mediated immunity. T cells are involved in the adaptive immune system. In some embodiments, T cells of the present disclosure can be any type of T cells, including, without limitation, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of one or more chimeric receptors, such as a chimeric TCRs or CARs.

Natural killer (NK) cells can be lymphocytes that are part of cell-mediated immunity and act during the innate immune response. NK cells do not need prior activation in order to perform their cytotoxic effect on target cells.

In some embodiments, an immunoresponsive cell of the present disclosure is a T cell. T cells of the present disclosure may be autologous, allogeneic, or derived in vitro from engineered progenitor or stem cells.

In some embodiments, an immunoresponsive cell of the present disclosure is a universal T cell with deficient TCR-αβ. Methods of developing universal T cells are described in the art, for example, in Valton et al., Molecular Therapy (2015); 23 9, 1507-1518, and Torikai et al., Blood 2012 119:5697-5705.

In some embodiments, an immunoresponsive cell of the present disclosure is an isolated immunoresponsive cell comprising one or more chimeric receptors of the present disclosure. In some embodiments, the immunoresponsive cell comprises one or more, two or more, three or more, four or more, five or more, six or more, seven or more, eight or more, nine or more, or ten or more chimeric receptors of the present disclosure.

In some embodiments, an immunoresponsive cell is a T cell. In some embodiments, an immunoresponsive cell is a Natural Killer (NK) cell.

In some embodiments, an immunoresponsive cell express or is capable of expressing an immune receptor. Immune receptors generally are capable of inducing signal transduction or changes in protein expression in the immune receptor-expressing cell that results in the modulation of an immune response upon binding to a cognate ligand (e.g., regulate, activate, initiate, stimulate, increase, prevent, attenuate, inhibit, reduce, decrease, inhibit, or suppress an immune response). For example, when CD3 chains present in a TCR/CAR cluster in response to ligand binding, an immunoreceptor tyrosine-based activation motifs (ITAMs)-meditated signal transduction cascade is produced. Specifically, in certain embodiments, when an endogenous TCR, exogenous TCR, chimeric TCR, or a CAR (specifically an activating CAR) binds their respective antigen, a formation of an immunological synapse occurs that includes clustering of many molecules near the bound receptor (e.g., CD4 or CD8, CD3γ/δ/ε/ζ, etc.). This clustering of membrane bound signaling molecules allows for ITAM motifs contained within the CD3 chains to become phosphorylated that in turn can initiate a T cell activation pathway and ultimately activates transcription factors, such as NF-κB and AP-1. These transcription factors are capable of inducing global gene expression of the T cell to increase IL-2 production for proliferation and expression of master regulator T cell proteins in order to initiate a T cell mediated immune response, such as cytokine production and/or T cell mediated killing.

### Cells Expressing Multiple Chimeric Receptors

In some embodiments, a cell of the present disclosure (e.g., an immunoresponsive cell) comprises two or more chimeric receptors of the present disclosure. In some embodiments, the cell comprises two or more chimeric receptors, wherein one of the two or more chimeric receptors is a chimeric inhibitory receptor. In some embodiments, the cell comprises three or more chimeric receptors, wherein at least one of the three or more chimeric receptors is a chimeric inhibitory receptor. In some embodiments, the cell comprises four or more chimeric receptors, wherein at least one of the four or more chimeric receptors is a chimeric inhibitory receptor. In some embodiments, the cell comprises five or more chimeric receptors, wherein at least one of the five or more chimeric receptors is a chimeric inhibitory receptor.

In some embodiments, each of the two or more chimeric receptors comprise a different antigen-binding domain, e.g., that binds to the same antigen or to a different antigen. In some embodiments each antigen bound by the two or more chimeric receptors are expressed on the same cell, such as an epithelial cell type (e.g., same epithelial cell type).

In embodiments where a cell of the present disclosure (e.g., an immunoresponsive cell) expresses two or more distinct chimeric receptors, the antigen-binding domain of each of the different chimeric receptors may be designed such that the antigen-binding domains do not interact with one another. For example, a cell of the present disclosure (e.g., an immunoresponsive cell) expressing a first chimeric receptor and a second chimeric receptor may comprise a first chimeric receptor that comprises an antigen-binding domain that does not form an association with the antigen-binding domain of the second chimeric receptor. For example, the antigen-binding domain of the first chimeric receptor may comprise an antibody fragment, such as an scFv, while the antigen-binding domain of the second chimeric receptor may comprise a VHH.

Without wishing to be bound by theory, it is believed that in cells having a plurality of chimeric membrane embedded receptors that each comprise an antigen-binding domain, interactions between the antigen-binding domains of each of the receptors can be undesirable, because such interactions may inhibit the ability of one or more of the antigen-binding domains to bind their cognate antigens. Accordingly, in embodiments where cells of the present disclosure (e.g., immunoresponsive cells) express two or more chimeric receptors, the chimeric receptors comprise antigen-binding domains that minimize such inhibitory interactions. In one embodiment, the antigen-binding domain of one chimeric receptor comprises an scFv and the antigen-binding domain of the second chimeric receptor comprises a single VH domain, e.g., a camelid, shark, or lamprey single VH domain, or a single VH domain derived from a human or mouse sequence.

In some embodiments, when present on the surface of a cell, binding of the antigen-binding domain of the first chimeric receptor to its cognate antigen is not substantially reduced by the presence of the second chimeric receptor. In some embodiments, binding of the antigen-binding domain of the first chimeric receptor to its cognate antigen in the presence of the second chimeric receptor is 85%, 90%, 95%, 96%, 97%, 98%, or 99% of binding of the antigen-binding domain of the first chimeric receptor to its cognate antigen in the absence of the second chimeric receptor. In some embodiments, when present on the surface of a cell, the antigen-binding domains of the first chimeric receptor and the second chimeric receptor associate with one another less than if both were scFv antigen-binding domains. In some embodiments, the antigen-binding domains of the first chimeric receptor and the second chimeric receptor associate with one another 85%, 90%, 95%, 96%, 97%, 98%, or 99% less than if both were scFv antigen-binding domains.

### Chimeric Inhibitory Receptors

In some embodiments, a cell of the present disclosure (e.g., an immunoresponsive cell) comprises one or more chimeric inhibitory receptors of the present disclosure. In some embodiments, each of the one or more chimeric inhibitory receptors comprises an antigen-binding domain that binds an antigen generally expressed on normal cells (e.g., cells generally considered to be healthy) but not on tumor cells. In some embodiments, a chimeric inhibitory receptor includes an antigen-binding domain that binds a target antigen.

In some embodiments, the one or more chimeric inhibitory receptors bind antigens that are expressed on a non-tumor cell derived from a tissue selected from brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor may be used, for example, with one or more activating chimeric receptors (e.g., activating chimeric TCRs or CARs) expressed on a cell of the present disclosure (e.g., an immunoresponsive cell) as NOT-logic gates to control, modulate, or otherwise inhibit one or more activities of the one or more activating chimeric receptors. In some embodiments, a chimeric inhibitory receptor of the present disclosure may inhibit one or more activities of a cell of the present disclosure (e.g., an immunoresponsive cell).

### Co-Stimulatory Ligands

In some embodiments, a cell of the present disclosure (e.g., an immunoresponsive cell) can further include one or more recombinant or exogenous co-stimulatory ligands. For example, the cell can be further transduced with one or more co-stimulatory ligands, such that the cell co-expresses or is induced to co-express one or more chimeric receptors of the present disclosure and one or more co-stimulatory ligands. Without wishing to be bound by theory, it is believed that the interaction between the one or more chimeric receptors and the one or more co-stimulatory ligands may provide a non-antigen-specific signal important for full activation of the cell. Examples of suitable co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Examples of suitable TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14). The immunoglobulin (Ig) superfamily is a large group of cell surface and soluble proteins that are involved in the recognition, binding, or adhesion processes of cells. These proteins share structural features with immunoglobulins and possess an immunoglobulin domain (fold). Examples of suitable immunoglobulin superfamily ligands include, without limitation, CD80 and CD86, both ligands for CD28, PD-L1/(B7-H1) that ligands for PD-1. In certain embodiments, the one or more co-stimulatory ligands are selected from 4-1BBL, CD80, CD86, CD70, OX40L, CD48, TNFRSF14, PD-L1, and combinations thereof.

### Chemokine Receptor

In some embodiments, a cell of the present disclosure (e.g., an immunoresponsive cell) comprises one or more chimeric receptors and may further include one or more chemokine receptors. For example, transgenic expression of chemokine receptor CCR2b or CXCR2 in cells, such as T cells, enhances trafficking to CCL2-secreting or CXCL1-secreting solid tumors (Craddock et al, J Immunother. 2010 October; 33 (8): 780-8 and Kershaw et al. Hum Gene Ther. 2002 Nov. 1; 13 (16): 1971-80). Without wishing to be bound by theory, it is believed that chemokine receptors expressed on chimeric receptor-expressing cells of the present disclosure may recognize chemokines secreted by tumors and improve targeting of the cell to the tumor, which may facilitate the infiltration of the cell to the tumor and enhance the antitumor efficacy of the cell. Chemokine receptors of the present disclosure may include a naturally occurring chemokine receptor, a recombinant chemokine receptor, or a chemokine-binding fragment thereof. Examples of suitable chemokine receptors that may expressed on a cell of the present disclosure include, without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In some embodiments, the chemokine receptor to be expressed on the cell is chosen based on the chemokines secreted by the tumor.

### Chimeric Receptor Regulation

Some embodiments of the present disclosure relate to regulating one or more chimeric receptor activities of chimeric receptor-expressing cells of the present. There are several ways chimeric receptor activities can be regulated. In some embodiments, a regulatable chimeric receptor, wherein one or more chimeric receptor activities can be controlled, may be desirable to optimize the safety and/or efficacy of the chimeric receptor therapy. For example, inducing apoptosis using a caspase fused to a dimerization domain (see, e.g., Di et al., N Engl. J. Med. 2011 Nov. 3; 365 (18): 1673-1683) can be used as a safety switch in the chimeric receptor therapy. In some embodiments, a chimeric receptor-expressing cell of the present disclosure can also express an inducible Caspase-9 (iCaspase-9) that, upon administration of a dimerizer drug, such as rimiducid (IUPAC name: [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate), induces activation of the Caspase-9 and results in apoptosis of the cells. In some embodiments, the iCaspase-9 contains a binding domain that comprises a chemical inducer of dimerization (CID) that mediates dimerization in the presence of the CID, which results in inducible and selective depletion of the chimeric receptor-expressing cells.

Alternatively, in some embodiments a chimeric receptor of the present disclosure may be regulated by utilizing a small molecule or an antibody that deactivates or otherwise inhibits chimeric receptor activity. For example, an antibody may delete the chimeric receptor-expressing cells by inducing antibody dependent cell-mediated cytotoxicity (ADCC). In some embodiments, a chimeric receptor-expressing cell of the present disclosure may further express an antigen that is recognized by a molecule that is capable of inducing cell death by ADCC or complement-induced cell death. For example, a chimeric receptor-expressing cell of the present disclosure may further express a receptor capable of being targeted by an antibody or antibody fragment. Examples of suitable receptors that may be targeted by an antibody or antibody fragment include, without limitation, EpCAM, VEGFR, integrins (e.g., ανβ3, α4, αI¾β3, α4β7, α5β1, ανβ3, αν), members of the TNF receptor superfamily (e.g., TRAIL-RI and TRAIL-R2), PDGF receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof.

In some embodiments, a chimeric receptor-expressing cell of the present disclosure may also express a truncated epidermal growth factor receptor (EGFR) that lacks signaling capacity but retains an epitope that is recognized by molecules capable of inducing ADCC (e.g., WO2011/056894).

In some embodiments, a chimeric receptor-expressing cell of the present disclosure further includes a highly expressing compact marker/suicide gene that combines target epitopes from both CD32 and CD20 antigens in the chimeric receptor-expressing cell, which binds an anti-CD20 antibody (e.g., rituximab) resulting in selective depletion of the chimeric receptor-expressing cell by ADCC. Other methods for depleting chimeric receptor-expressing cells of the present disclosure my include, without limitation, administration of a monoclonal anti-CD52 antibody that selectively binds and targets the chimeric receptor-expressing cell for destruction by inducing ADCC. In some embodiments, the chimeric receptor-expressing cell can be selectively targeted using a chimeric receptor ligand, such as an anti-idiotypic antibody. In some embodiments, the anti-idiotypic antibody can cause effector cell activity, such as ADCC or ADC activity. In some embodiments, the chimeric receptor ligand can be further coupled to an agent that induces cell killing, such as a toxin. In some embodiments, a chimeric receptor-expressing cell of the present disclosure may further express a target protein recognized by a cell depleting agent of the present disclosure. In some embodiments, the target protein is CD20 and the cell depleting agent is an anti-CD20 antibody. In such embodiments, the cell depleting agent is administered once it is desirable to reduce or eliminate the chimeric receptor-expressing cell. In some embodiments, the cell depleting agent is an anti-CD52 antibody.

In some embodiments, a regulated chimeric receptor comprises a set of polypeptides, in which the components of a chimeric receptor of the present disclosure are partitioned on separate polypeptides or members. For example, the set of polypeptides may include a dimerization switch that, when in the presence of a dimerization molecule, can couple the polypeptides to one another to form a functional chimeric receptor.

### Chimeric Receptor-Encoding Polynucleotide Constructs

Certain aspects of the present disclosure relate to polynucleotides (e.g., isolated polynucleotides) encoding one or more chimeric receptors of the present. In some embodiments, the polynucleotide is an RNA construct, such as a messenger RNA (mRNA) transcript or a modified RNA. In some embodiments, the polynucleotide is a DNA construct.

In some embodiments, a polynucleotide of the present disclosure encodes a chimeric receptor that comprises one or more antigen-binding domain, where each domain binds to a target antigen, a transmembrane domain, and one or more intracellular signaling domains. In some embodiments, the polynucleotide encodes a chimeric receptor that comprises an antigen-binding domain, a transmembrane domain, a primary signaling domain (e.g., CD3-zeta domain), and one or more costimulatory signaling domains. In some embodiments, the polynucleotide further comprises a nucleic acid sequence encoding a spacer region. In some embodiments, the antigen-binding domain is connected to the transmembrane domain by the spacer region. In some embodiments, the spacer region comprises a nucleic acid sequence selected from any of the nucleic acid sequences listed in Table 3. In some embodiments, the nucleic acid further comprises a nucleotide sequence encoding a leader sequence.

The polynucleotides of the present disclosure may be obtained using any suitable recombinant methods known in the art, including, without limitation, by screening libraries from cells expressing the gene of interest, by deriving the gene of interest from a vector known to include the gene, or by isolating the gene of interest directly from cells and tissues containing the gene using standard techniques. Alternatively, the gene of interest may be produced synthetically.

In some embodiments, a polynucleotide of the present disclosure in comprised within a vector. In some embodiments, a polynucleotide of the present disclosure is expressed in a cell via transposons, a CRISPR/Cas9 system, a TALEN, or a zinc finger nuclease.

In some embodiments, expression of a polynucleotide encoding a chimeric receptor of the present disclosure may be achieved by operably linking the nucleic acid to a promoter and incorporating the construct into an expression vector. A suitable vector can replicate and integrate in eukaryotic cells. Typical cloning vectors contain transcription and translation terminators, initiation sequences, and promoters useful for regulating expression of the desired nucleic acid.

In some embodiments, expression constructs of the present disclosure may also be used for nucleic acid immunization and gene therapy, using standard gene delivery protocols (e.g., U.S. Pat. Nos. 5,399,346, 5,580,859, and 5,589,466). In some embodiments, a vector of the present disclosure is a gene therapy vector.

A polynucleotide of the present disclosure can be cloned into a number of types of vectors. For example, the polynucleotide can be cloned into a vector including, without limitation, a plasmid, a phagemid, a phage derivative, an animal virus, or a cosmid. In some embodiments, the vector may be an expression vector, a replication vector, a probe generation vector, or a sequencing vector.

In some embodiments, the plasmid vector comprises a transposon/transposase system to incorporate the polynucleotides of the present disclosure into the host cell genome. Methods of expressing proteins in immune cells using a transposon and transposase plasmid system are generally described in Chicaybam L, Hum Gene Ther. 2019 April; 30 (4): 511-522. doi: 10.1089/hum.2018.218; and Ptáčková P, Cytotherapy. 2018 April; 20 (4): 507-520. doi: 10.1016/j.jcyt.2017.10.001, each of which are hereby incorporated by reference in their entirety. In some embodiments, the transposon system is the Sleeping Beauty transposon/transposase or the piggyBac transposon/transposase.

In some embodiments, an expression vector of the present disclosure may be provided to a cell in the form of a viral vector. Suitable viral vector systems are well known in the art. For example, viral vectors may be derived from retroviruses, adenoviruses, adeno-associated viruses, herpes viruses, and lentiviruses. In some embodiments, a vector of the present disclosure is a lentiviral vector. Lentiviral vectors are suitable for long-term gene transfer as such vectors allow long-term, stable integration of a transgene and its propagation in daughter cells. Lentiviral vectors are also advantageous over vectors derived from onco-retroviruses (e.g., murine leukemia viruses) in that lentiviral vectors can transduce non-proliferating cells. In some embodiments, a vector of the present disclosure is an adenoviral vector (A5/35). In some embodiments, a vector of the present disclosure contains an origin of replication functional in at least one organism, a promoter sequence, convenient restriction endonuclease sites, and one or more selectable markers (e.g., WO01/96584; WO01/29058; and U.S. Pat. No. 6,326,193). A number of viral based systems have been developed for gene transfer into mammalian cells. A selected gene can be inserted into a vector and packaged in retroviral particles using techniques known in the art. The recombinant virus can then be isolated and delivered to mammalian cells either in vivo or ex vivo. A number of retroviral systems are known in the art.

In some embodiments, vectors of the present disclosure include additional promoter elements, such as enhancers that regulate the frequency of transcriptional initiation. Enhancers are typically located in a region that is 30 bp to 110 bp upstream of the start site, although a number of promoters have been shown to contain functional elements downstream of the start site as well. The spacing between promoter elements may be flexible, so that promoter function is preserved when elements are inverted or moved relative to one another. For example, in the thymidine kinase (tk) promoter the spacing between promoter elements can be increased to 50 bp apart before activity begins to decline. Depending on the promoter, individual elements may function either cooperatively or independently to activate transcription. Exemplary promoters may include, without limitation, the SFFV gene promoter, the EFS gene promoter, the CMV IE gene promoter, the EFla promoter, the ubiquitin C promoter, and the phosphoglycerokinase (PGK) promoter.

In some embodiments, a promoter that is capable of expressing a polynucleotide of the present disclosure in a mammalian cell, such as an immunoresponsive cell of the present disclosure, is the EFla promoter. The native EFla promoter drives expression of the alpha subunit of the elongation factor-1 complex, which is responsible for the enzymatic delivery of aminoacyl tRNAs to the ribosome. The EFla promoter has been widely used in mammalian expression plasmids and has been shown to be effective in driving chimeric receptor expression from polynucleotide cloned into a lentiviral vector.

In some embodiments, a promoter that is capable of expressing a polynucleotide of the present disclosure in a mammalian cell, such as an immunoresponsive cell of the present disclosure, is a constitutive promoter. For example, a suitable constitutive promoter is the spleen focus forming virus (SFFV) promoter. Another example of a suitable constitutive promoter is the immediate early cytomegalovirus (CMV) promoter. The CMV promoter is a strong constitutive promoter that is capable of driving high levels of expression of any polynucleotide sequence operatively linked to the promoter. Other suitable constitutive promoters include, without limitation, a ubiquitin C (UbiC) promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, an actin promoter, a myosin promoter, an elongation factor-la promoter, a hemoglobin promoter, and a creatine kinase promoter.

In some embodiments, a promoter that is capable of expressing a polynucleotide of the present disclosure in a mammalian cell, such as an immunoresponsive cell of the present disclosure, is an inducible promoter. Use of an inducible promoter may provide a molecular switch that is capable of inducing or repressing expression of a polynucleotide of the present disclosure when the promoter is operatively linked to the polynucleotide. Examples of inducible promoters include, without limitation, a metallothionine promoter, a glucocorticoid promoter, a progesterone promoter, and a tetracycline promoter.

In some embodiments, a vector of the present disclosure may further comprise a signal sequence to facilitate secretion, a polyadenylation signal and transcription terminator, an element allowing episomal replication, and/or elements allowing for selection.

In some embodiments, a vector of the present disclosure can further comprise a selectable marker gene and/or reporter gene to facilitate identification and selection of chimeric receptor-expressing cells from a population of cells that have been transduced with the vector. In some embodiments, the selectable marker may be encoded by a polynucleotide that is separate from the vector and used in a co-transfection procedure. Either selectable marker or reporter gene may be flanked with appropriate regulator sequences to allow expression in host cells. Examples of selectable markers include, without limitation, antibiotic-resistance genes, such as neo and the like.

In some embodiments, reporter genes may be used for identifying transduced cells and for evaluating the functionality of regulatory sequences. As disclosed herein, a reporter gene is a gene that is not present in or expressed by the recipient organism or tissue and that encodes a polypeptide whose expression results in an easily detectable property, such as enzymatic activity. Expression of the reporter gene can be assayed at a suitable time after the polynucleotide has been introduced into the recipient cells. Examples of reporter genes include, without limitation, genes encoding for luciferase, genes encoding for beta-galactosidase, genes encoding for chloramphenicol acetyl transferase, genes encoding for secreted alkaline phosphatase, and genes encoding for green fluorescent protein. Suitable expression systems are well known in the art and may be prepared using known techniques or obtained commercially. In some embodiments, a construct with a minimal 5′ flanking region showing the highest level of expression of the reporter gene is identified as the promoter. Such promoter regions may be linked to a reporter gene and used to evaluate agents for the ability to modulate promoter-driven transcription.

In some embodiments, a vector comprising a polynucleotide sequence encoding a chimeric receptor of the present disclosure further comprises a second polynucleotide encoding a polypeptide that increases the activity of the chimeric receptor.

In embodiments where a chimeric receptor-expressing cell comprises two or more chimeric receptors, a single polynucleotide may encode the two or more chimeric receptors under a single regulatory control element (e.g., promoter) or under separate regulatory control elements for each chimeric receptor-encoding nucleotide sequence comprised in the polynucleotide. In some embodiments where a chimeric receptor-expressing cell comprises two or more chimeric receptors, each chimeric receptor may be encoded by a separate polynucleotide. In some embodiments, each separate polynucleotide comprises its own control element (e.g., promoter). In some embodiments, a single polynucleotide encodes the two or more chimeric receptors and the chimeric receptor-encoding nucleotide sequences are in the same reading frame and are expressed as a single polypeptide chain. In such embodiments, the two or more chimeric receptors may be separated by one or more peptide cleavage sites, such as auto-cleavage sites or substrates for an intracellular protease. Suitable peptide cleavage sites may include, without limitation, a T2A peptide cleavage site, a P2A peptide cleavage site, an E2A peptide cleavage sire, and an F2A peptide cleavage site. In some embodiments, the two or more chimeric receptors comprise a T2A peptide cleavage site. In some embodiments, the two or more chimeric receptors comprise an E2A peptide cleavage site. In some embodiments, the two or more chimeric receptors comprise a T2A and an E2A peptide cleavage site.

Methods of introducing and expressing genes into a cell are well known in the art. For example, in some embodiments, an expression vector can be transferred into a host cell by physical, chemical, or biological means. Examples of physical means for introducing a polynucleotide into a host cell include, without limitation, calcium phosphate precipitation, lipofection, particle bombardment, microinjection, and electroporation. Examples of chemical means for introducing a polynucleotide into a host cell include, without limitation, colloidal dispersion systems, macromolecule complexes, nanocapsules, microspheres, beads, and lipid-based systems including oil-in-water emulsions, micelles, mixed micelles, and liposomes. Examples of biological means for introducing a polynucleotide into a host cell include, without limitation, the use of DNA and RNA vectors.

In some embodiments, liposomes may be used as a non-viral delivery system to introduce a polynucleotide or vector of the present disclosure into a host cell in vitro, ex vivo, or in vivo. In some embodiments, the polynucleotide may be associated with a lipid, for example by being encapsulated in the aqueous interior of a liposome, being interspersed within the lipid bilayer of a liposome, being attached to a liposome via a linking molecule that is associated with both the liposome and the polynucleotide, being entrapped in a liposome, being complexed with a liposome, being dispersed in a solution containing a lipid, being mixed with a lipid, being combined with a lipid, being contained as a suspension in a lipid, being contained or complexed with a micelle, or otherwise being associated with a lipid. As disclosed herein, lipid-associated polynucleotide or vector compositions are not limited to any particular structure in solution. In some embodiments, such compositions may be present in a bilayer structure, as micelles or with a “collapsed” structure. Such compositions may also be interspersed in a solution, forming aggregates that are not uniform in size or shape. As disclosed herein, lipids are fatty substances that may be naturally occurring or synthetic. In some embodiments, lipids can include the fatty droplets that naturally occur in the cytoplasm or the class of compounds that contain long-chain aliphatic hydrocarbons and their derivatives, such as fatty acids, alcohols, amines, amino alcohols, and aldehydes. Suitable lipids may be obtained from commercial sources and include, without limitation, dimyristyl phosphatidylcholine (“DMPC”), dicetylphosphate (“DCP”), cholesterol, and dimyristylphosphatidylglycerol (“DMPG”). Stock solutions of lipids in chloroform or chloroform/methanol can be stored at about −20° C. Chloroform is used as the solvent, as it is more readily evaporated than methanol. As used herein, a “liposome” may encompass a variety of single and multilamellar lipid vehicles formed by the generation of enclosed lipid bilayers or aggregates. In some embodiments, liposomes can be characterized as having vesicular structures with a phospholipid bilayer membrane and an inner aqueous medium. In some embodiments, multilamellar liposomes may have multiple lipid layers separated by aqueous medium. Multilamellar liposomes can form spontaneously when phospholipids are suspended in an excess of aqueous solution. In some embodiments, lipid components may undergo self-rearrangement before the formation of closed structures and can entrap water and dissolved solutes between the lipid bilayers. In some embodiments, the lipids may assume a micellar structure or merely exist as nonuniform aggregates of lipid molecules.

In some embodiments, a polynucleotide or vector of the present disclosure is introduced into a mammalian host cell, such as an immunoresponsive cell of the present disclosure. In some embodiments, the presence of a polynucleotide or vector of the present disclosure in a host cell may be confirmed by any suitable assay known in the art, including without limitation Southern blot assays, Northern blot assays, RT-PCR, PCR, ELISA assays, and Western blot assays.

In some embodiments, a polynucleotide or vector of the present disclosure is stably transduced into an immunoresponsive cell of the present disclosure. In some embodiments, cells that exhibit stable expression of the polynucleotide or vector express the encoded chimeric receptor for at least 1 week, at least 2 weeks, at least 3 weeks, at least 4 weeks, at least 5 weeks, at least 6 weeks, at least 7 weeks, at least 8 weeks, at least 3 months, at least 6 months, at least 9 months, or at least 12 months after transduction.

In embodiments where a chimeric receptor of the present disclosure is transiently expressed in a cell, a chimeric receptor-encoding polynucleotide or vector of the present disclosure is transfected into an immunoresponsive cell of the present disclosure. In some embodiments the immunoresponsive cell expresses the chimeric receptor for about 4 days, about 5 days, about 6 days, about 7 days, about 8 days, about 9 days, about 10 days, about 11 days, about 12 days, about 13 days, about 14 days, or about 15 days after transfection.

In some embodiments, the polynucleotide construct encodes a bicistronic chimeric antigen receptor. In some embodiments, the polynucleotide construct encodes a bivalent chimeric antigen receptor.

### Pharmaceutical Compositions and Administration

Certain aspects of the present disclosure relate to compositions (e.g., pharmaceutical compositions) comprising one or more chimeric receptors of the present disclosure or immunoresponsive cells of the present disclosure that express such one or more chimeric receptors. In some embodiments, compositions comprising chimeric receptors or genetically modified immunoresponsive cells that express such chimeric receptors can be provided systemically or directly to a subject for the treatment of a proliferative disorder, such as a myeloid disorder.

### Methods of Treatment

Certain aspects of the present disclosure relate to methods of using the chimeric receptors and genetically modified cells of the present disclosure (e.g., immunoresponsive cells) that express such chimeric receptors to treat subjects in need thereof. In some embodiments, the methods of the present disclosure are useful for treating cancer in a subject, such as solid tumor.

As disclosed herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

### Therapeutic Treatment

In some embodiments, the methods of the present disclosure increase an immune response in a subject in need thereof. In some embodiments, the subject is a human.

In any and all aspects of increasing an immune response as described herein, any increase or decrease or alteration of an aspect of characteristic(s) or function(s) is as compared to a cell not contacted with an immunoresponsive cell as described herein.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response.

### Combination Therapies

In some embodiments, genetically modified cells of the present disclosure (e.g., immunoresponsive cells) expressing one or more chimeric receptors of the present disclosure may be used in combination with other known agents and therapies. In some embodiments, a combination therapy of the present disclosure comprises a genetically modified cells of the present disclosure that can be administered in combination with one or more additional therapeutic agents. In some embodiments, the genetically modified cell and the one or more additional therapeutic agents can be administered simultaneously, in the same or in separate compositions, or sequentially. For sequential administration, the genetically modified can be administered first, and the one or more additional agents can be administered second, or the order of administration can be reversed. In some embodiments, the genetically modified cells are further modified to express one or more additional therapeutic agents.

### Kits

Certain aspects of the present disclosure relate to kits for the treatment and/or prevention of a cancer (e.g., solid tumors). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, a therapeutic or prophylactic composition is provided together with instructions for administering the therapeutic or prophylactic composition to a subject having or at risk of developing cancer (e.g., a solid tumor). In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### Enumerated Embodiments

Embodiment 1: A fusion protein comprising:
(a) an extracellular domain (ECD) comprising a cytokine;
(b) a hinge, and
(c) a transmembrane domain.

Embodiment 2: The fusion protein of claim 1, further comprising an intracellular domain (ICD) comprising an intracellular signaling domain derived from an activating immune cell receptor.

Embodiment 3: The fusion protein of embodiment 1 or 2, wherein the ECD comprises a cytokine selected from the group consisting of: IL-15, IL1-beta, IL-2, IL-4, IL-6, IL-7, IL-10, IL-12, IL-17A, IL-18, IL-21, IL-22, Type I interferons, Interferon-gamma, TNF-alpha, CCL21a, CXCL10, CXCL11, CXCL13, CCL19, CXCL9, XCL1, mutants thereof, fragments thereof, and fusion proteins thereof.

Embodiment 4: The fusion protein of any one of embodiments 1-3, wherein the cytokine is a chemokine.

Embodiment 5: The fusion protein of any one of embodiments 1-3, wherein the ECD comprises an IL-15.

Embodiment 6: The fusion protein of any one of embodiments 1-3 or 5, wherein the ECD comprises an IL-15 and an IL-15 receptor.

Embodiment 7: The fusion protein of any one of embodiments 1-3 or 5-6, wherein the ECD comprises an IL-15 and an IL-15Rα.

Embodiment 8: The fusion protein of any one of embodiments 1-3 or 5-7, wherein the ECD comprises an IL-15 and a sushi domain of IL-15Rα.

Embodiment 9: The fusion protein of any one of embodiments 1-8, further comprising a signal sequence.

Embodiment 10: The fusion protein of embodiment 9, wherein the signal sequence comprises a CD8 signal sequence or an IgE signal sequence.

Embodiment 11: The fusion protein of embodiment 9 or 10, wherein the signal sequence comprises the amino acid sequence of SEQ ID NO:70.

Embodiment 12: The fusion protein of any one of embodiments 8-11, wherein the IL-15 and the sushi domain of IL-15Rα are connected via a linker.

Embodiment 13: The fusion protein of embodiment 12, wherein the linker comprises a glycine-serine linker.

Embodiment 14: The fusion protein of embodiment 12 or 13, wherein the linker comprises the amino acid sequence of SEQ ID NO: 15.

Embodiment 15: The fusion protein of any one of embodiments 8-14, wherein the IL-15 comprises the amino acid sequence of SEQ ID NO:1.

Embodiment 16: The fusion protein of any one of embodiments 8-15, wherein the sushi domain of IL-15Rα comprises the amino acid sequence of SEQ ID NO:2.

Embodiment 17: The fusion protein of any one of embodiments 8-16, wherein the ECD comprises the amino acid sequence of SEQ ID NO:85.

Embodiment 18: The fusion protein of any one of embodiments 8-17, wherein the ECD does not comprise a non-sushi domain of IL-15Rα.

Embodiment 19: The fusion protein of any one of embodiments 8-18, wherein the ECD further comprises the amino acid sequence of SEQ ID NO:104.

Embodiment 20: The fusion protein of any one of embodiments 1-19, wherein the transmembrane domain comprises a transmembrane domain derived from an activating immune cell receptor.

Embodiment 21: The fusion protein of any one of embodiments 1-20, wherein the transmembrane domain comprises a transmembrane domain from a membrane protein selected from the group consisting of: CD25, CD7, CD3zeta, CD4, 4-1BB, ICOS, CTLA-4, LAX, LAT, PD-1, LAG-3, TIM3, LIR-1 KIR3DS1, KIR3DL1, NKG2D, NKG2A, TIGIT, BTLA, IL-15Rα, CD28, OX40, CD8a, NKp46, and 2B4.

Embodiment 22: The fusion protein of embodiment 1-21, wherein the transmembrane domain comprises an IL-15Rα transmembrane domain.

Embodiment 23: The fusion protein of embodiments 21 or 22, wherein the IL-15Rα transmembrane domain comprises the amino acid sequence of SEQ ID NO:5.

Embodiment 24: The fusion protein of embodiment 1-21, wherein the transmembrane domain comprises a CD28 transmembrane domain.

Embodiment 25: The fusion protein of embodiments 21 or 24, wherein the CD28 transmembrane domain comprises the amino acid sequence of SEQ ID NO:7.

Embodiment 26: The fusion protein of embodiment 1-21, wherein the transmembrane domain comprises an OX40 transmembrane domain.

Embodiment 27: The fusion protein of embodiments 21 or 26, wherein the OX40 transmembrane domain comprises the amino acid sequence of SEQ ID NO:9.

Embodiment 28: The fusion protein of embodiment 1-21, wherein the transmembrane domain comprises a CD8 transmembrane domain.

Embodiment 29: The fusion protein of embodiments 21 or 28, wherein the CD8 transmembrane domain comprises the amino acid sequence of SEQ ID NO:36.

Embodiment 30: The fusion protein of embodiment 1-21, wherein the transmembrane domain comprises an NKp46 transmembrane domain.

Embodiment 31: The fusion protein of embodiments 21 or 30, wherein the NKp46 transmembrane domain comprises the amino acid sequence of SEQ ID NO:12.

Embodiment 32: The fusion protein of embodiment 1-21, wherein the transmembrane domain comprises a 2B4 transmembrane domain.

Embodiment 33: The fusion protein of embodiments 21 or 32, wherein the 2B4 transmembrane domain comprises the amino acid sequence of SEQ ID NO:14.

Embodiment 34: The fusion protein of any one of embodiments 2-33, wherein the transmembrane domain and the ICD are derived from the same activating immune cell receptor.

Embodiment 35: The fusion protein of any one of embodiments 2-33, wherein the transmembrane domain and the ICD are derived from different activating immune cell receptors.

Embodiment 36: The fusion protein of any one of embodiments 1-35, wherein the hinge is derived from an activating immune cell receptor.

Embodiment 37: The fusion protein of any one of embodiments 1-36, wherein the hinge is derived from a protein selected from the group consisting of: IgG4, IgG2, IgD, KIR2DS2, LNGFR, PDGFR, CD28, CD8a, and MAG.

Embodiment 38: The fusion protein of any one of embodiments 1-37, wherein the hinge is derived from CD28.

Embodiment 39: The fusion protein of any one of embodiments 1-38, wherein the hinge comprises the amino acid sequence of SEQ ID NO:40.

Embodiment 40: The fusion protein of any one of embodiments 1-37, wherein the hinge is derived from CD8a.

Embodiment 41: The fusion protein of any one of embodiments 1-37 and 40, wherein the hinge comprises the amino acid sequence of SEQ ID NO:49.

Embodiment 42: The fusion protein of any one of embodiments 1-37, wherein the hinge is derived from MAG.

Embodiment 43: The fusion protein of any one of embodiments 1-37 and 42, wherein the hinge comprises the amino acid sequence of SEQ ID NO:3.

Embodiment 44: The fusion protein of any one of embodiments 2-43, wherein the hinge and the ICD are derived from the same activating immune cell receptor.

Embodiment 45: The fusion protein of any one of embodiments 2-43, wherein the hinge and the ICD are derived from different activating immune cell receptors.

Embodiment 46: The fusion protein of any one of embodiments 2-45, wherein the ICD does not comprise an IL-15Rα intracellular domain.

Embodiment 47: The fusion protein of any one of embodiments 2-46, wherein the ICD comprises a signaling domain derived from a membrane protein selected from the group consisting of: CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

Embodiment 48: The fusion protein of any one of embodiments 2-47, wherein the ICD comprises an intracellular signaling domain derived from CD28.

Embodiment 49: The fusion protein of any one of embodiments 2-47 and 48, wherein the ICD comprises the amino acid sequence of SEQ ID NO:18.

Embodiment 50: The fusion protein of any one of embodiments 2-47, wherein the ICD comprises an intracellular signaling domain derived from OX40.

Embodiment 51: The fusion protein of any one of embodiments 2-47 and 50, wherein the ICD comprises the amino acid sequence of SEQ ID NO:64.

Embodiment 52: The fusion protein of any one of embodiments 2-47, wherein the ICD comprises an intracellular signaling domain derived from 4-1BB.

Embodiment 53: The fusion protein of any one of embodiments 2-47 and 52, wherein the ICD comprises the amino acid sequence of SEQ ID NO:66.

Embodiment 54: The fusion protein of any one of embodiments 2-47, wherein the ICD comprises an intracellular signaling domain derived from NKp46.

Embodiment 55: The fusion protein of any one of embodiments 2-47 and 54, wherein the ICD comprises the amino acid sequence of SEQ ID NO:67.

Embodiment 56: The fusion protein of any one of embodiments 2-47, wherein the ICD comprises an intracellular signaling domain derived from 2B4.

Embodiment 57: The fusion protein of any one of embodiments 2-47 and 56, wherein the ICD comprises the amino acid sequence of SEQ ID NO:69.

Embodiment 58: The fusion protein of any one of embodiments 1-55, wherein the fusion protein comprises, from N-terminus to C-terminus, an IgE signal sequence, the ECD, the hinge, the transmembrane domain, and an ICD.

Embodiment 59: A nucleotide construct encoding the fusion protein of any one of embodiments 1-58.

Embodiment 60: A vector comprising the nucleotide construct of embodiment 59.

Embodiment 61: An immunoresponsive cell comprising the fusion protein of any one of embodiments 1-58, the nucleotide construct of embodiment 59, or the vector of embodiment 60.

Embodiment 62: The immunoresponsive cell of embodiment 61, wherein the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

Embodiment 63: The immunoresponsive cell of embodiment 61 or 62, wherein the immunoresponsive cell is a T cell.

Embodiment 64: The immunoresponsive cell of embodiment 61 or 62, wherein the immunoresponsive cell is an NK cell.

Embodiment 65: The immunoresponsive cell of any one of embodiments 61-64, wherein the immunoresponsive cell further comprises an activating immune receptor.

Embodiment 66: The immunoresponsive cell of any one of embodiments 61-65, wherein the immunoresponsive cell further comprises a chimeric antigen receptor (CAR).

Embodiment 67: The immunoresponsive cell of embodiment 66, wherein the CAR comprises:
a. an antigen-binding domain specific for a target antigen,
b. a transmembrane domain, and
c. an intracellular signaling domain (ICD).

Embodiment 68: The immunoresponsive cell of embodiment 67, wherein the target antigen is a tumor-associated antigen, a bacterial antigen, a viral antigen, or a self-antigen.

Embodiment 69: The immunoresponsive cell of any one of embodiments 66-68, wherein the CAR comprises a transmembrane domain derived from an activating immune cell receptor.

Embodiment 70: The immunoresponsive cell of any one of embodiments 66-69, wherein the CAR comprises an ICD of an activating immune cell receptor.

Embodiment 71: The immunoresponsive cell of embodiment 70, wherein the ICD of the CAR is different from the ICD of the fusion protein.

Embodiment 72: The immunoresponsive cell of embodiment 70, wherein the ICD of the CAR is the same as the ICD of the fusion protein.

Embodiment 73: The immunoresponsive cell of any one of embodiments 66-72, wherein the CAR comprises a CD3zeta ICD.

Embodiment 74: The immunoresponsive cell of any one of embodiments 66-73, wherein the CAR does not comprise a co-stimulatory domain.

Embodiment 75: The immunoresponsive cell of any one of embodiments 61-74, wherein a costimulatory signal is provided from the fusion protein.

Embodiment 76: The immunoresponsive cell of any one of embodiments 61-75, wherein the immunoresponsive cell is allogeneic.

Embodiment 77: The immunoresponsive cell of any one of embodiments 61-75, wherein the immunoresponsive cell is autologous.

Embodiment 78: A pharmaceutical composition comprising the fusion protein of any one of embodiments 1-58, the vector of embodiment 60, or the immunoresponsive cell of any one of embodiments 61-77, and a pharmaceutically acceptable carrier, pharmaceutically acceptable excipient, or a combination thereof.

Embodiment 79: A method of treating a subject in need thereof, the method comprising administering to a subject a therapeutically effective dose of the fusion protein of any one of embodiments 1-58, the immunoresponsive cell of any one of embodiments 61-77, or the pharmaceutical composition of embodiment 78.

Embodiment 80: A method of stimulating an immune response in a subject, the method comprising administering to a subject a therapeutically effective dose of the fusion protein of any one of embodiments 1-58, the immunoresponsive cell of any one of embodiments 61-77, or the pharmaceutical composition of embodiment 78.

Embodiment 81: A method of reducing tumor volume in a subject, the method comprising administering to a subject having a tumor a composition comprising the fusion protein of any one of embodiments 1-58, the immunoresponsive cell of any one of embodiments 61-77, or the pharmaceutical composition of embodiment 78.

Embodiment 82: A method of providing an anti-tumor immunity in a subject, the method comprising administering to a subject in need thereof a therapeutically effective dose of the fusion protein of any one of embodiments 1-58, the immunoresponsive cell of any one of embodiments 61-77, or the pharmaceutical composition of embodiment 78.

Embodiment 83: A method of treating a subject having cancer, the method comprising administering a therapeutically effective dose of the fusion protein of any one of embodiments 1-58, the immunoresponsive cell of any one of embodiments 61-77, or the pharmaceutical composition of embodiment 78.

Embodiment 84: A kit for treating and/or preventing a tumor, comprising the fusion protein of any one of embodiments 1-58.

Embodiment 85: The kit of embodiment 84, wherein the kit further comprises written instructions for using the fusion protein for treating and/or preventing a tumor in a subject.

Embodiment 86: A kit for treating and/or preventing a tumor, comprising the immunoresponsive cell of any one of embodiments 61-77.

Embodiment 87: The kit of embodiment 86, wherein the kit further comprises written instructions for using the immunoresponsive cell for treating and/or preventing a tumor in a subject.

Embodiment 88: A kit for treating and/or preventing a tumor, comprising the pharmaceutical composition of embodiment 78.

Embodiment 89: The kit of embodiment 88, wherein the kit further comprises written instructions for using the pharmaceutical composition for treating and/or preventing a tumor in a subject.

### EXAMPLES

The following are examples of methods and compositions of the present disclosure. It is understood that various other embodiments may be practiced, given the general description provided herein.

Below are examples of specific embodiments for carrying out the claimed subject matter of the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1: Generation and Characterization of IL-15/IL-15Rα Fusion Proteins

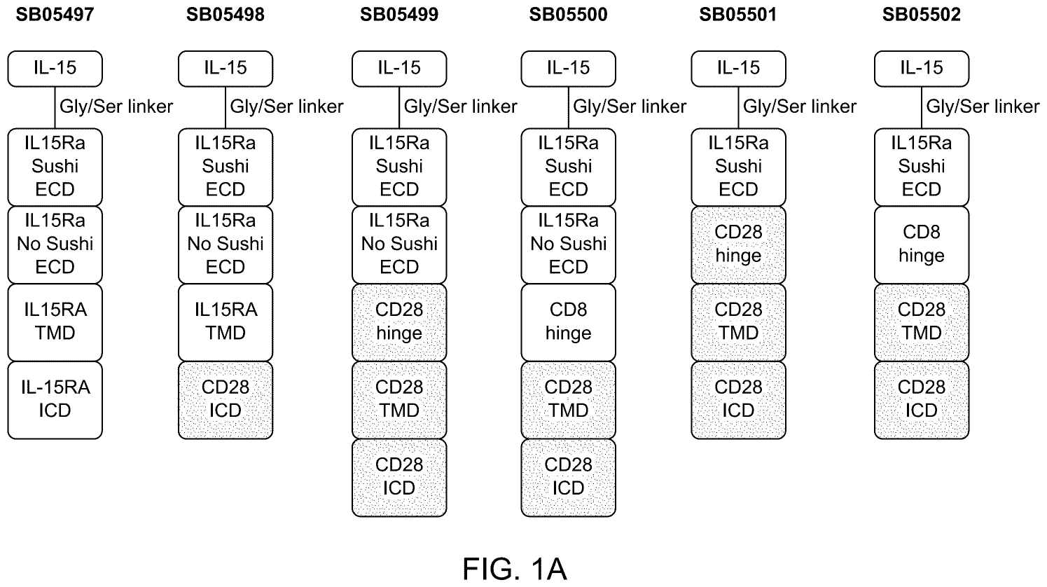

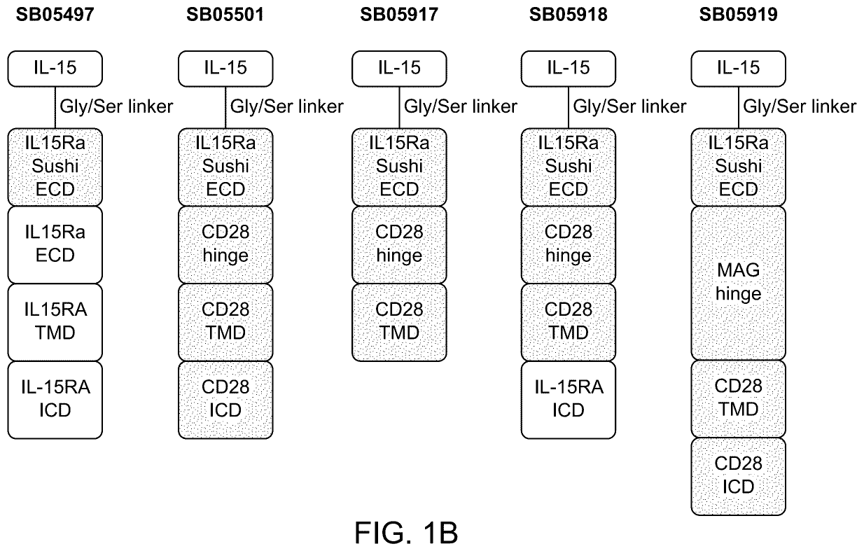

To generate new IL-15/IL 15Rα molecular constructs, using molecular cloning techniques known in the art, various IL-15Rα constructs were designed with different transmembrane domains, hinges, and/or an intracellular signaling domains (e.g., CD28) (as shown in FIG. 1A) fused to IL-15 and an IL-15Rα sushi extracellular domain. The fusion proteins comprise the IL-15/IL-15Rα sushi extracellular domain and a heterologous intracellular signaling domain, resulting in the ability to produce two cellular signals. The amino acid sequences of these fusion proteins are provided in Table 5.

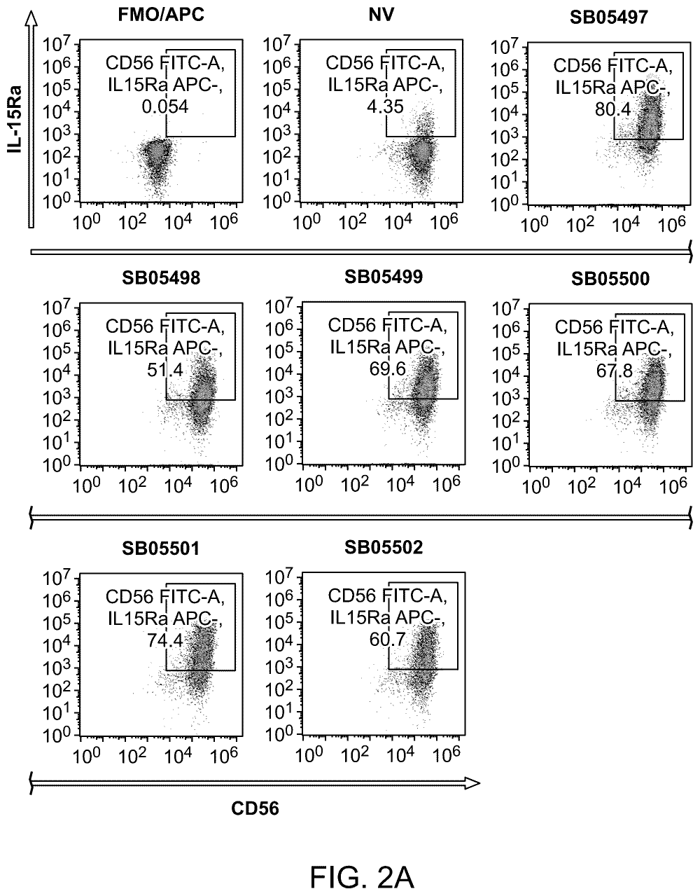

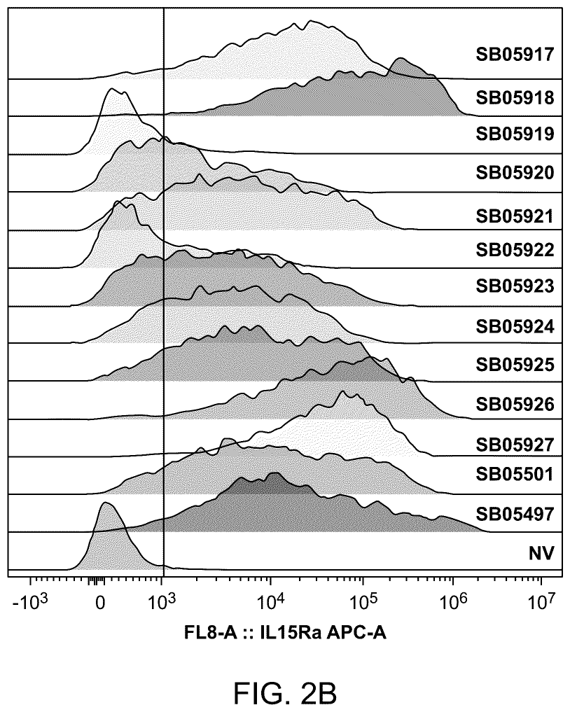

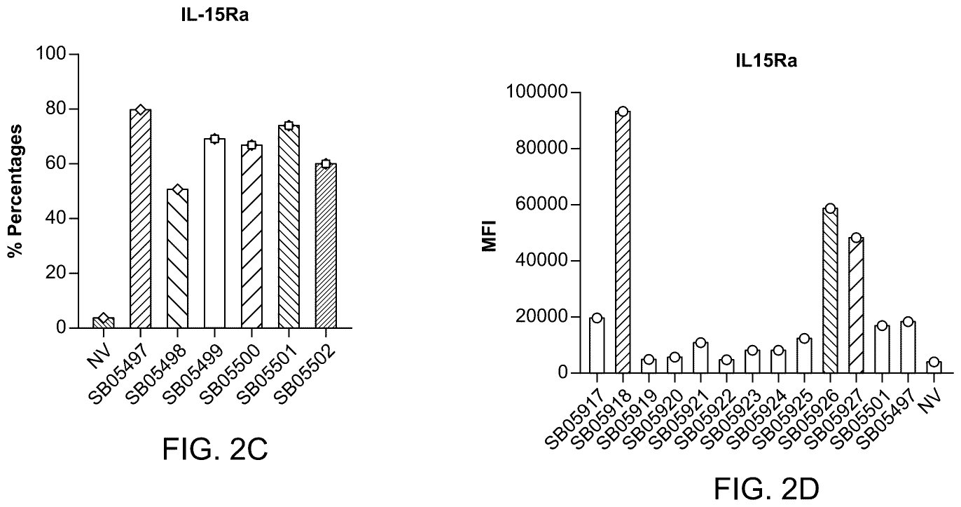

NK cells were isolated from apheresis pack derived peripheral blood mononuclear cells (PBMCs), depleted of CD3+ cells by affinity-selection, and enriched for CD56+ cells by affinity-selection. Resultant NK cells were transduced with 150 μl of virus for expression of fusion protein. Two days after transduction, 100,000 transduced-NK cells were immunofluorescently stained to detect surface expression of IL-15Rα. Expression was assessed by flow cytometry, as shown in FIG. 2A, distribution is shown in FIG. 2B, and percentage of expressing cells is shown in FIG. 2C. Mean fluorescent intensity (MFI) is shown in FIG. 2D. As shown, a majority of cells expressed the IL-15Rα fusion proteins.

### Analysis of Signaling in Cytokine-Withdrawal

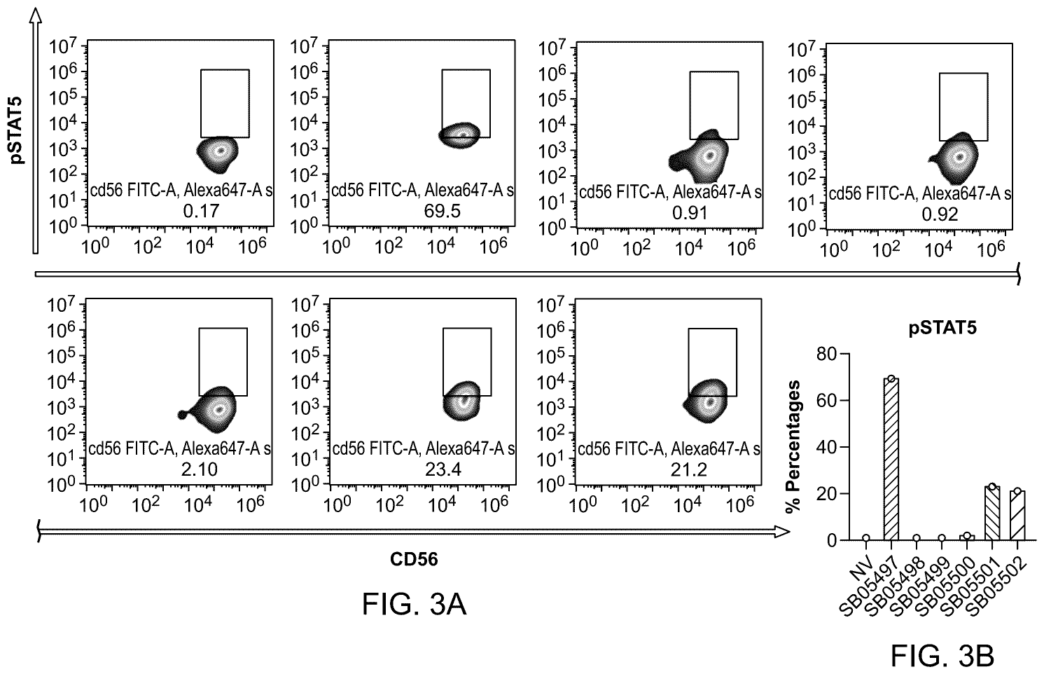

For phospho-Stat5 determination, untransduced and transduced NK cells were washed twice with NK MACS media plus 5% human serum (depleted of IL-2 and rhIL-15), and cytokine-starved for 18 hours. Cells were centrifuged for 5 minutes at 1500 rpm, washed once with FACS buffer, and stained with a FITC-conjugated anti-CD56 antibody for 30 min at 4° C. and then fixed for 10 minutes at room temperature using the BD Phosphoflow Lysis/Fix buffer (BD Biosciences). The cells were washed once in PBS and permeabilized using the BD Phosphoflow Perm buffer III (BD Biosciences) at −20° C. for 30 minutes. Cells were washed twice with FACS buffer and stained with AF647-conjugated anti-phospho-Stat5 for 30 minutes at 4° C. Expression was assessed by flow cytometry, as shown in FIG. 3A, and percentage of pSTAT5-expressing cells is shown in FIG. 3B.

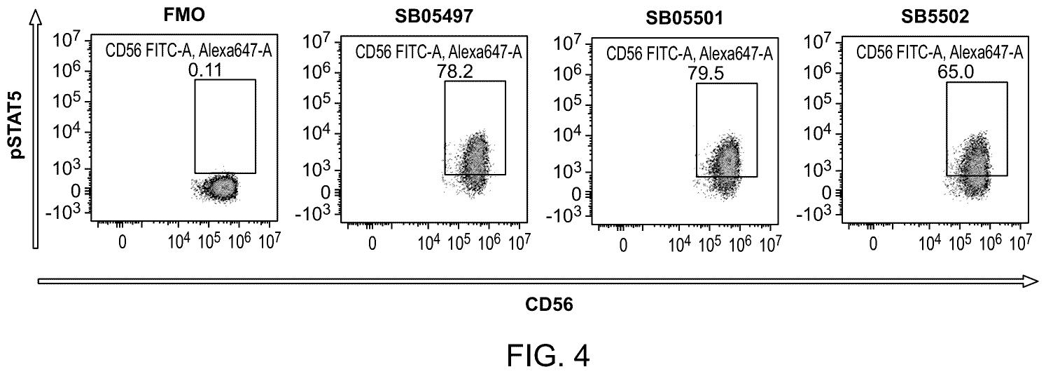

NK cells transduced with at least 3e9 infectious units (IU) (calculated via copies of Vector Genomes—VGs) and were cytokine-starved for 6 days. 6 days after cytokine withdrawal, cells were centrifuged for 5 minutes at 1500 rpm, washed once with FACS buffer, stained with a FITC-conjugated anti-CD56 antibody for 30 min at 4° C. and then fixed for 10 minutes at room temperature using the BD Phosphoflow Lysis/Fix buffer (BD Biosciences). The cells were washed once in PBS and permeabilized using the BD Phosphoflow Perm buffer III (BD Biosciences) at −20° C. for 30 minutes. Cells were washed twice with FACS buffer and stained with AF647-conjugated anti-phospho-Stat5 for 30 minutes at 4° C. Expression was assessed by flow cytometry, as shown in FIG. 4.

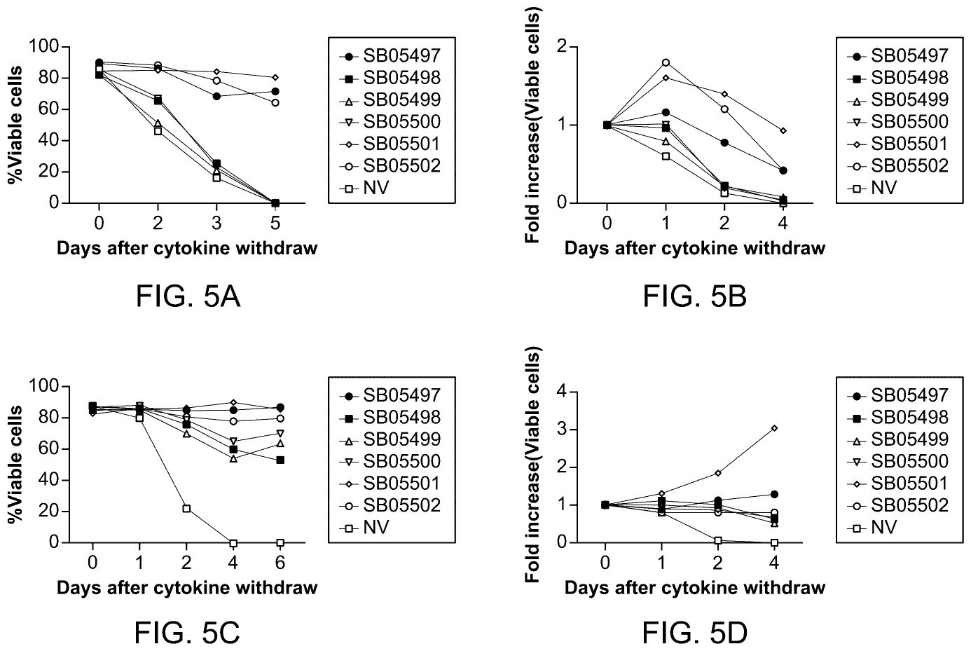

1 million NK cells were transduced with 150 μl of virus. Untransduced and transduced NK cells were washed twice with base NK MACS media plus 5% human serum. 1 million each of transduced and untransduced NK cells were starved of cytokines in 2 ml of NK MACS media, plus 5% human serum for 5 days. Cell viability and cell count after cytokine withdrawal were determined using the Celleca MX cell counter (percent viable cells shown in FIG. 5A, fold increase in viable cells shown in FIG. 5B).

Similarly, 1 million NK cells were transduced with at least 3e9 IU (calculated via copies of Vector Genomes—VGs). 48 hours after transduction, untransduced and transduced NK cells were washed twice with base NK MACS media plus 5% human serum. 1 million each of transduced and untransduced NK cells were starved of cytokines in 2 ml of NK MACS media plus 5% human serum for 6 days. Cell viability and cell count after cytokine withdrawal were performed using the Celleca MX cell counter (percent viable cells shown in FIG. 5C, fold increase in viable cells shown in FIG. 5D).

Expression of the IL-15/IL-15Rα fusion proteins in cytokine-starved NK cells provided surprisingly enhanced proliferation and viability as compared to the control IL-15/IL-15Rα proteins having both an IL-15Rα ICD and transmembrane domain.

Analysis of Signaling with Cytokine Addition

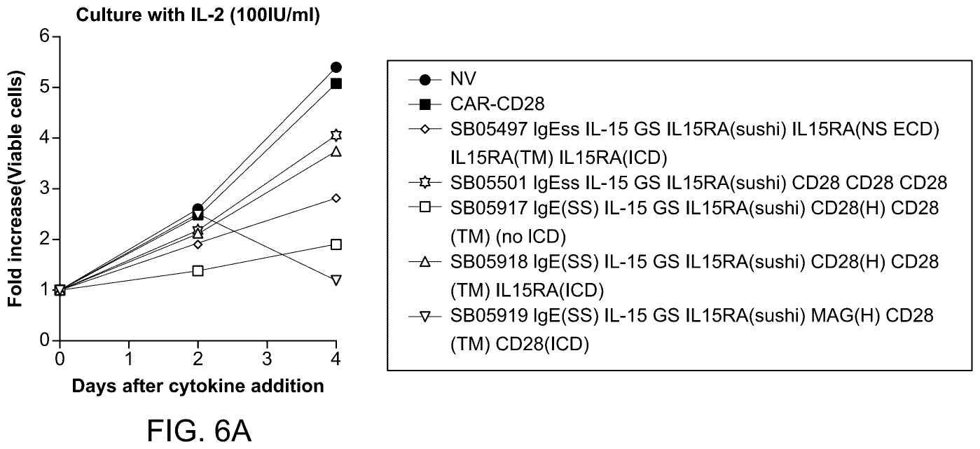

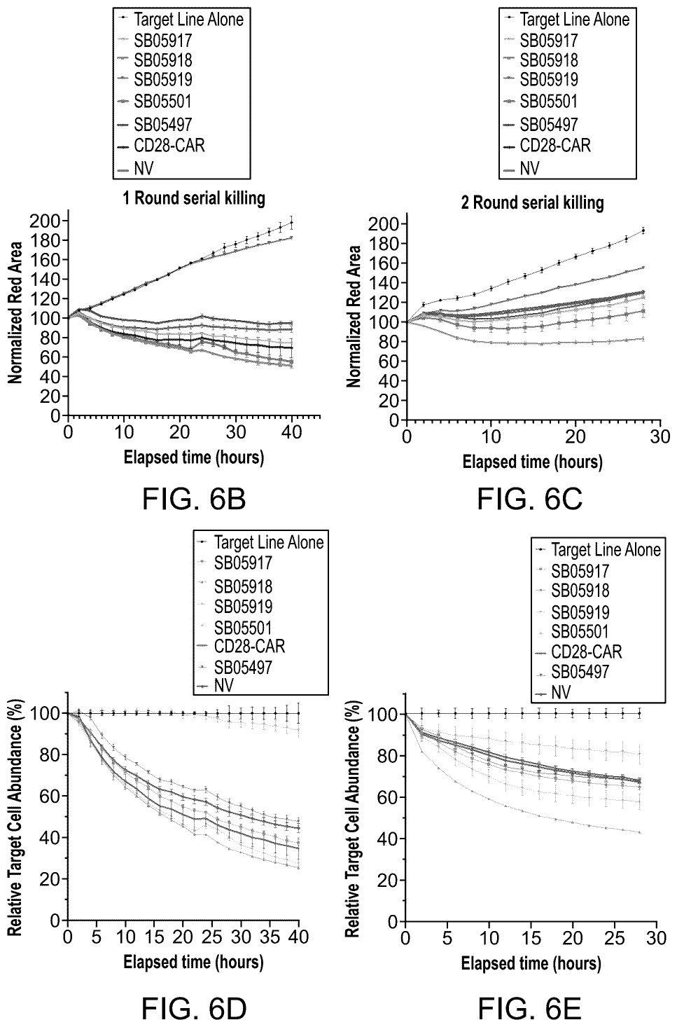

To assess killing induced by NK cells expressing the fusion proteins, untransduced and transduced NK cells were cultured as described above. NK cells expressing CD28 CAR was used as a control. IL-2 was added about 72 hours in culture at a concentration of 100 IU/mL. Cells were counted two days, and again four days, after IL-2 stimulation (shown in FIG. 6A). At four days, NK cells were cultured with target cells at a 1:1 ratio to determine killing by the NK cells. Normalized results are shown in FIG. 6B and FIG. 6C, and relative target cell abundance is shown in FIG. 6D and FIG. 6E. It was observed that the ICD of CD28 was required for SB05501 enhanced killing and serial killing activity. SB05918 displayed enhanced expansion and serial killing activity as compared to SB05501, suggesting the CD28 hinge and transmembrane domains enhance IL-15/IL-15Rα signaling. SB05918 displayed enhanced proliferation and serial killing activity as compared to SB05497. Without wishing to be bound by theory, it is hypothesized that SB05918 allows for stabilization of the IL-15/IL-15Rα interaction with CD122, without affecting intrinsic proliferation and killing activity of the cells expressing said fusion protein.

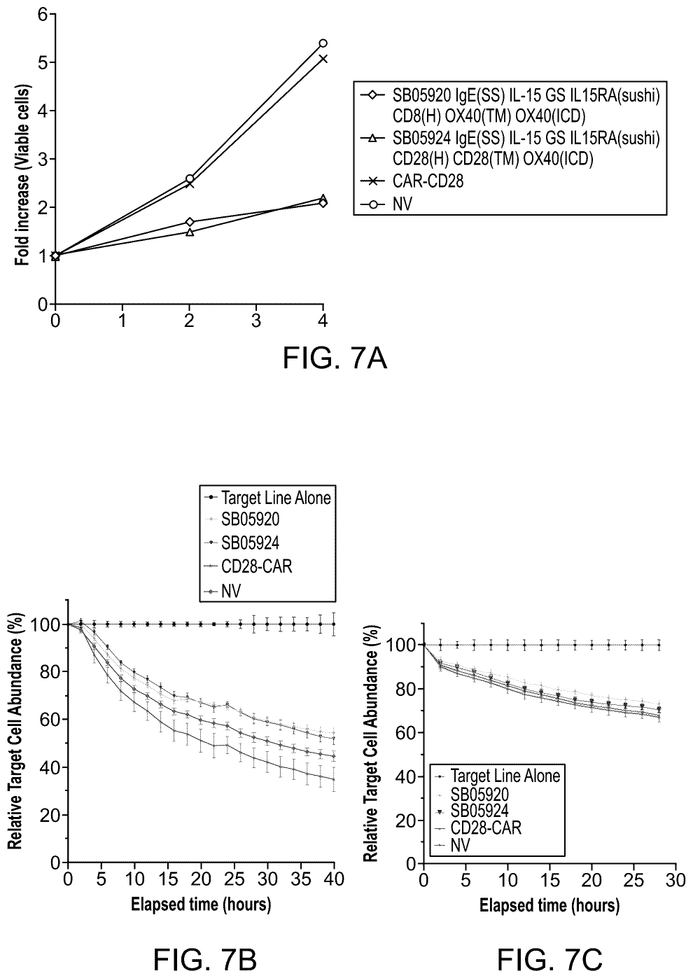

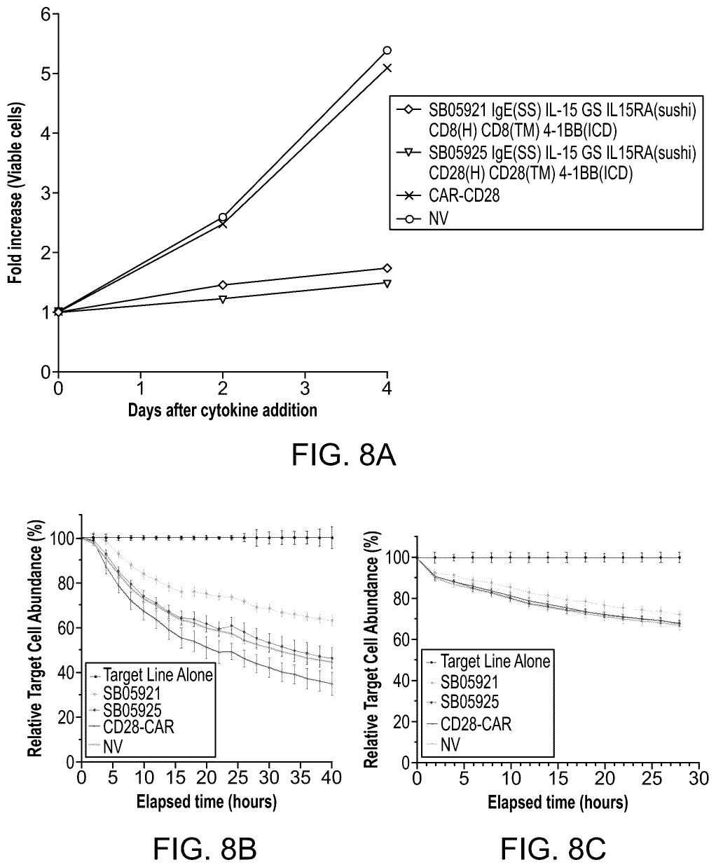

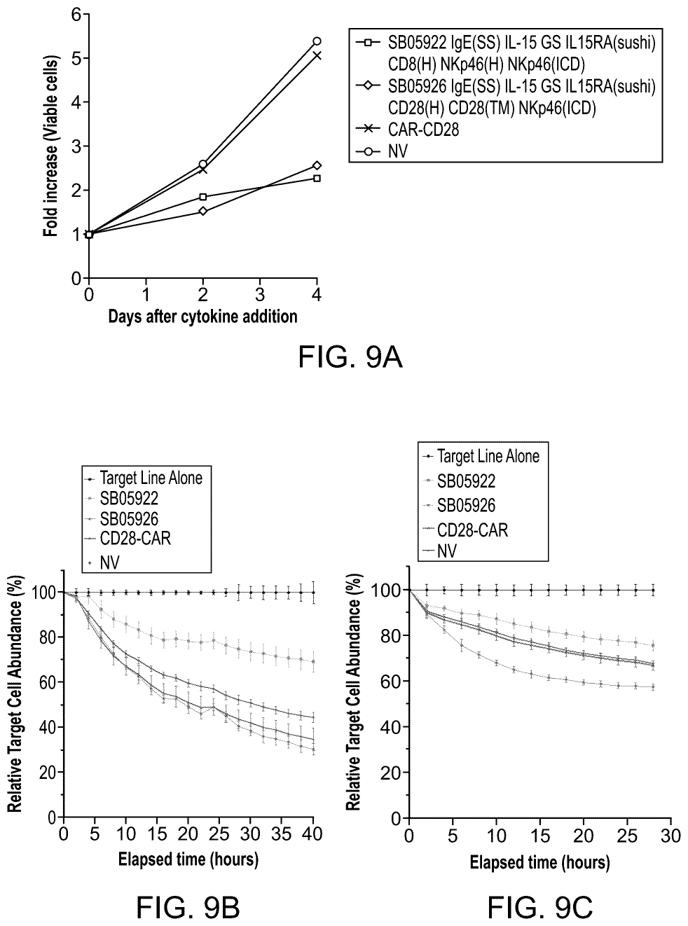

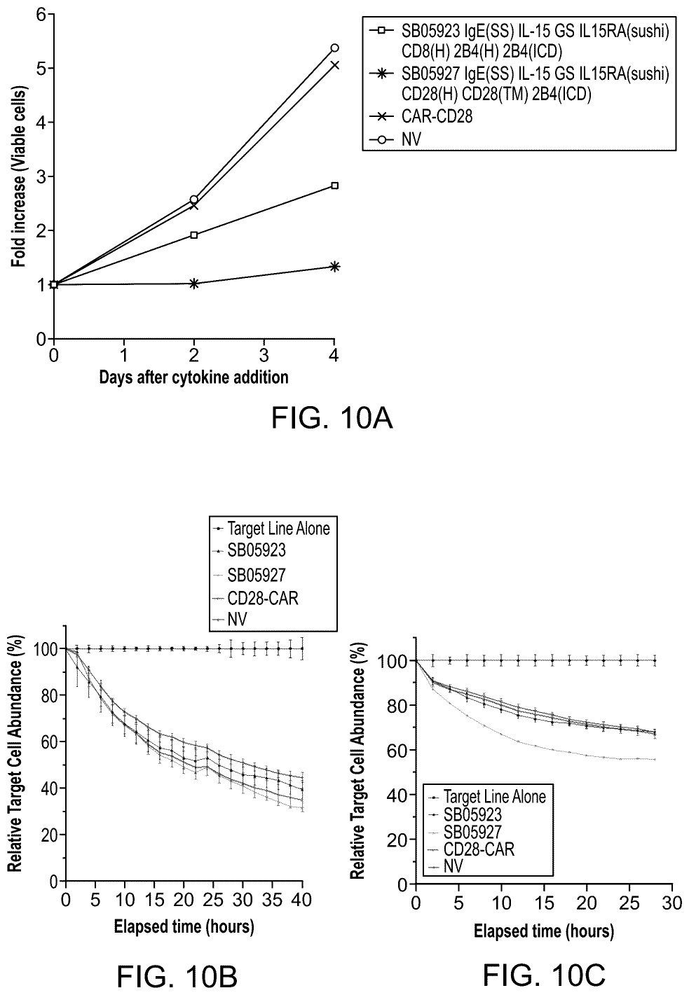

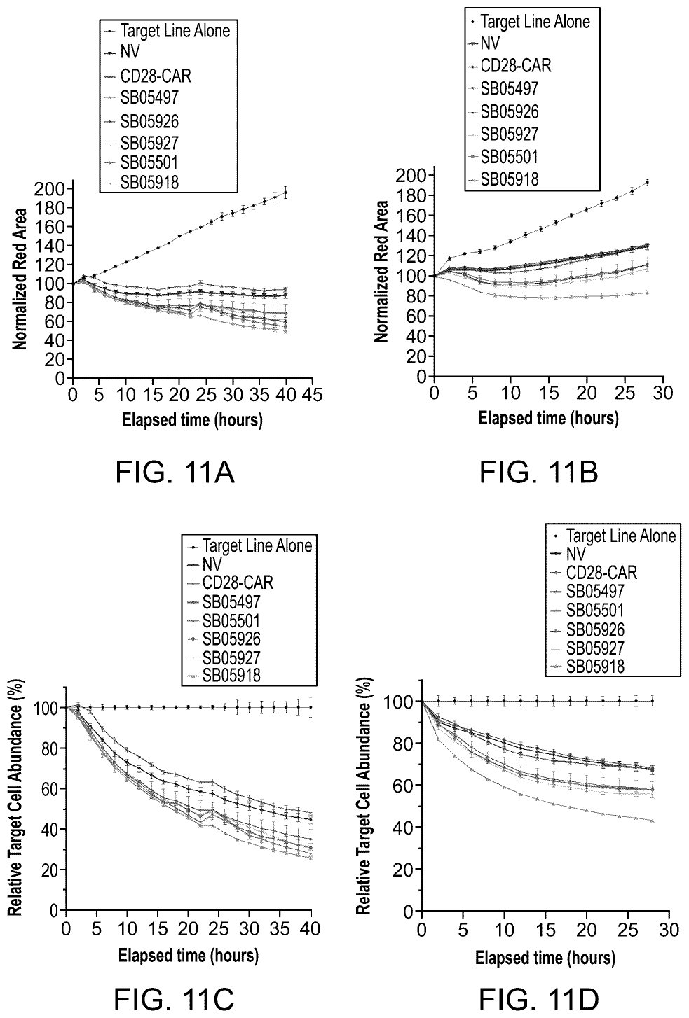

To compare fusion proteins expressing a CD8 hinge and a CD28 hinge, SB05920 and SB05924, both expressing an ICD of OX40, were compared in expansion (FIG. 7A) and serial killing assays (FIGS. 7B-7C), conducted as described above. SB05921 and SB05925, both expressing an ICD of 4-1BB, were also compared in expansion (FIG. 8A) and serial killing assays (FIGS. 8B-8C), SB05922 and SB05926, both expressing an ICD of NKp46, were also compared in expansion (FIG. 9A) and serial killing assays (FIGS. 9B-9C), and SB05923 and SB05927, both expressing an ICD of 2B4, were also compared in expansion (FIG. 10A) and serial killing assays (FIGS. 10B-10C). All comparative results are summarized in FIGS. 11A-11B (normalized results) and FIGS. 11C-11D (relative target cell abundance).

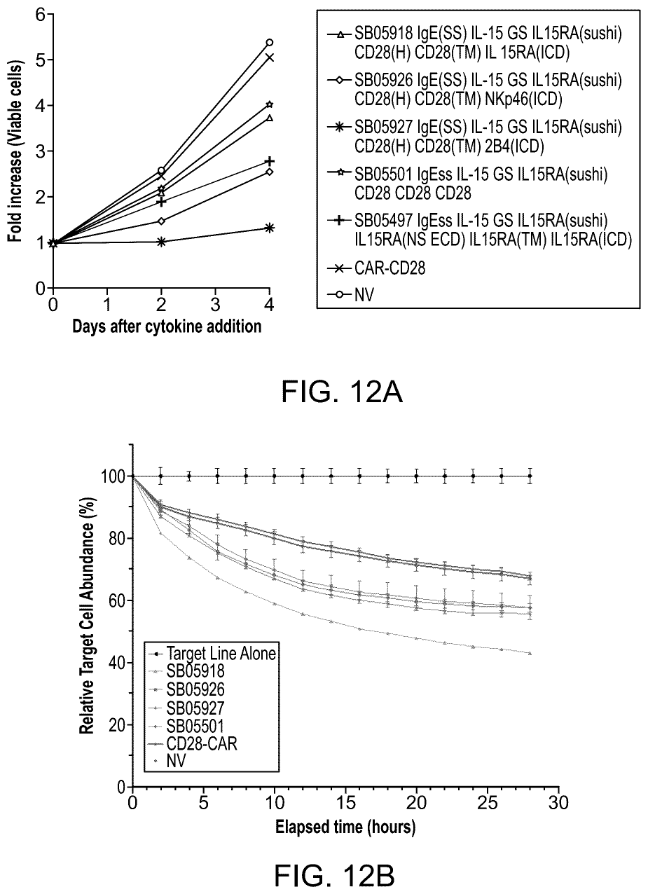

A comparison of IL-15Rα expression (FIG. 2D) to fold increase in viable cells (FIG. 12A) suggests high levels of IL-15Rα do not directly correlate with high levels of expansion when cultured with IL-2. The fusion protein with highest IL-15Rα expression, SB05918, did provide a significant increase in serial killing functionality (FIG. 12B).

Surprisingly, it was observed that the most effective fusion proteins have hinge and transmembrane regions from CD28, suggesting these domains lead to improvement of NK cell functionality.

### Example 2: Assessment of IL-15/IL-15Rα Function in NK Cells

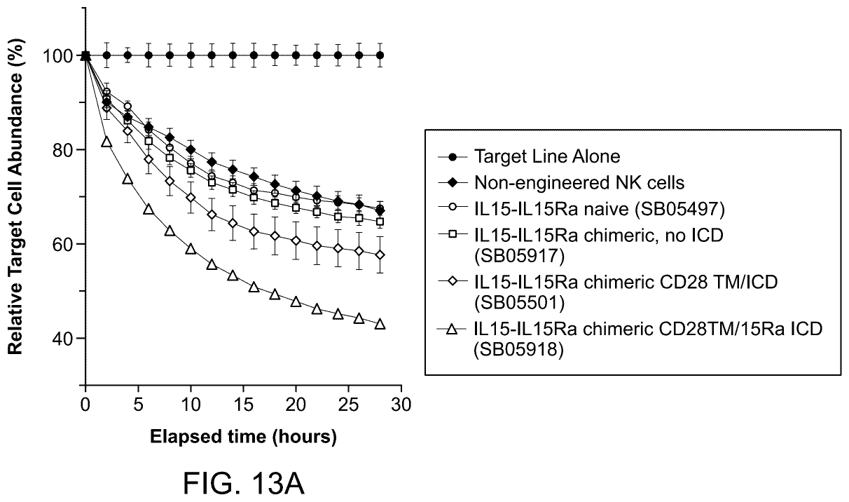

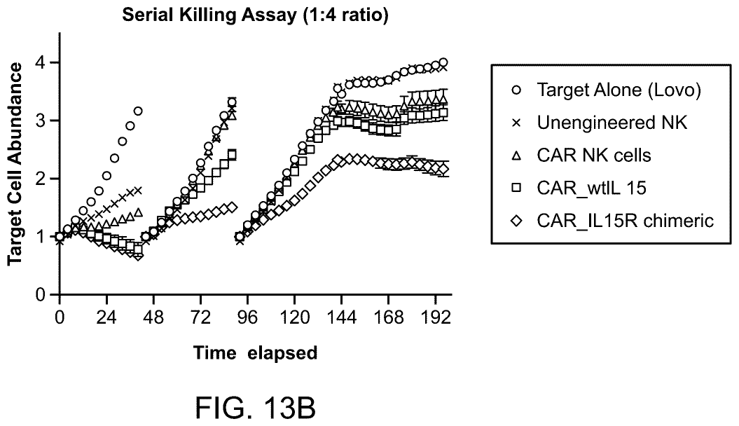

To assess the chimeric cytokine receptor (IL15-IL 15Rα chimeric proteins) with a NK cells, this example demonstrates the efficacy of cell killing of different chimeric cytokine receptor architectures (FIG. 13A). NK cell killing is assessed by expressing a chimeric antigen receptor in the presence of wild-type IL15 expression or the chimeric cytokine receptor (FIG. 13B).

To compare the efficacy of WT IL15, various constructions of a chimeric protein containing IL15 tethered to the chimeric IL15 receptor were tested. Ls174t cells were incubated with NK cells transduced to express IL15-IL15a (SB05497), IL15-IL15R-no ICD (SB05917), IL15-IL15R chimeric CD28 TM/ICD (SB05501), or IL15-IL15a chimeric CD28TM/15Ra ICD (SB05918). Target cell abundance was measured over the course of the study, The chimeric protein comprising CD28 TM/ICD domains and the CD28 TM/IL15Ra ICD yielded improved target cell killing compared to SB05497 and SB05917 (FIG. 13A).

NK cells were transduced with retrovirus encoding a CEA-activating CAR and a chimeric cytokine receptor (IL15-IL15Ra chimeric proteins; SB05918) or wild-type IL15. 6 to 7 days after transduction, NK cells were collected, counted and seeded in a co-culture with tumor target cells that constitutively express a fluorescent reporter (mKate) at the appropriate effector to target (E: T) ratios. Tumor target cell growth (area) was quantified using an imaging-based system (Incucyte, Sartorius). After 48 to 72 hours of co-culture, new target cells were plated, and the NK cells were collected from the original plates and transferred to newly seeded targets for a 2nd or 3rd tumor re-challenge (serial killing). Target cell area was quantified and compared across different conditions. After three rounds of killing at an E: T ratio of 1:4, the CAR-NKs expressing the chimeric IL15R fusion protein demonstrated improved cell killing of Lovo target cells compared to CAR-NKs not expressing the IL15Rα fusion protein or CAR-NKs expressing wild-type IL15 (FIG. 13B).

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1.-33. (canceled)

34. A fusion protein comprising a cytokine operably linked to at least a portion of a chimeric receptor, wherein the chimeric receptor comprises an extracellular domain (ECD), a hinge domain, a transmembrane domain and an intracellular domain (ICD); wherein at least one of the ECD, the hinge domain, the transmembrane domain, and the ICD is derived from a native cognate receptor of the cytokine; and wherein at least one other of the ECD, the hinge domain, the TM domain, and the ICD is derived from a different receptor, wherein the ECD is derived from the native cognate receptor of the cytokine and wherein the at least one of the hinge, TM, and/or ICD is derived from a different activating immune receptor.

35. The fusion protein of claim 34, wherein the cytokine is selected from the group consisting of: a chemokine, IL-15, IL1-beta, IL-2, IL-4, IL-6, IL-7, IL-10, IL-12, IL-17A, IL-18, IL-21, IL-22, Type I interferons, Interferon-gamma, TNF-alpha, CCL21a, CXCL10, CXCL11, CXCL13, CCL19, CXCL9, XCL1, mutants thereof, fragments thereof, and fusion proteins thereof.

36. The fusion protein of claim 34, wherein the cytokine is IL-15, and optionally wherein the ECD comprises (a) an IL-15 receptor ECD, (b) an IL-15Rα ECD, or (c) a sushi domain of IL-15Rα, optionally wherein the IL-15 is connected to the IL-15 receptor ECD, the IL15Rα ECD, or the sushi domain of IL-15Rα via a linker, optionally wherein the linker comprises a glycine serine linker.

37. The fusion protein of claim 34, wherein the ECD is selected from the group consisting of (a) an IL-15, (b) IL-15 and an IL-15 receptor, (c) an IL-15 and an IL-15Rα, (d) an IL-15 and a sushi domain of IL-15Rα, (e) an IL15 receptor ECD, and (f) an IL15Rα ECD.

38. The fusion protein of claim 37, further comprising a linker, optionally wherein the linker comprises a glycine serine linker, optionally wherein the IL-15 is connected to the IL-15 receptor, the IL15Rα, or the sushi domain of IL-15Rα via the linker, optionally wherein the linker comprises a glycine serine linker.

39. The fusion protein of claim 34, wherein the ECD comprises the IL-15 and the sushi domain of IL-15Rα, wherein the IL-15 and the sushi domain of IL-15Rα are connected via a linker, optionally wherein the fusion protein comprises the amino acid sequence of SEQ ID NO: 85, optionally wherein the ECD does not comprise a non-sushi domain of IL-15Rα, optionally wherein the non-sushi domain comprises the amino acid sequence of SEQ ID NO: 104.

40. The fusion protein of claim 34, wherein the ECD further comprises the amino acid sequence of SEQ ID NO:104.

41. The fusion protein of claim 34, further comprising a signal sequence, optionally wherein the signal sequence comprises a CD8 signal sequence or an IgE signal sequence.

42. The fusion protein of claim 34, wherein the transmembrane domain comprises a transmembrane domain derived from an activating immune cell receptor, optionally wherein the transmembrane domain comprises a transmembrane domain from a membrane protein selected from the group consisting of: CD25, CD7, CD3zeta, CD4, 4-1BB, ICOS, CTLA-4, LAX, LAT, PD-1, LAG-3, TIM3, LIR-1 KIR3DS1, KIR3DL1, NKG2D, NKG2A, TIGIT, BTLA, IL-15Rα, CD28, OX40, CD8a, NKp46, and 2B4.

43. The fusion protein of claim 34, wherein the hinge is derived from an activating immune cell receptor, optionally wherein the hinge is derived from a protein selected from the group consisting of: IgG4, IgG2, IgD, KIR2DS2, LNGFR, PDGFR, CD28, CD8a, and MAG.

44. The fusion protein of claim 34, wherein the ICD does not comprise an IL-15Rα intracellular domain.

45. The fusion protein of claim 34, wherein the ICD comprises a signaling domain derived from a membrane protein selected from the group consisting of: CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

46. The fusion protein of claim 34, wherein the fusion protein comprises, from N-terminus to C-terminus, an IgE signal sequence, the ECD, the hinge, the transmembrane domain, and an ICD.

47. A nucleotide construct encoding the fusion protein of claim 34.

48. A vector comprising the nucleotide construct of claim 47.

49. An immunoresponsive cell comprising the fusion protein of claim 34, optionally wherein the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

50. The immunoresponsive cell of claim 49, further comprising an activating immune receptor, and/or a chimeric antigen receptor (CAR), optionally wherein: the CAR does not comprise a co-stimulatory domain, and/or a costimulatory signal is provided from the fusion protein, and/or the immunoresponsive cell is allogeneic or autologous.

51. A pharmaceutical composition comprising the immunoresponsive cell of claim 49, and a pharmaceutically acceptable carrier, pharmaceutically acceptable excipient, or a combination thereof.

52. A method of treating a subject in need thereof, the method comprising administering to a subject a therapeutically effective dose of the immunoresponsive cell of claim 49.

53. A kit for treating and/or preventing a tumor, comprising the immunoresponsive cell of claim 49, wherein: (a) the kit further comprises written instructions for using the fusion protein for treating and/or preventing a tumor in a subject, or (b) the kit further comprises written instructions for using the immunoresponsive cell for treating and/or preventing a tumor in a subject, or (c) the kit further comprises written instructions for using the pharmaceutical composition for treating and/or preventing a tumor in a subject.


SEQUENCE LISTING (128 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 65 aa, synthetic construct
ITCPPPMSVEHADIWVKSYSLYSRERYICNSGFKRKAGTSSLTECVLNKATNVAHWTTPSLKCIR

SEQ ID NO: 3 - protein, 279 aa, Homo sapiens
YPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGP

SEQ ID NO: 4 - DNA, 837 bp, Homo sapiens
  source 1..837: mol_type = other DNA
taccctccagtgatcgtggaaatgaacagcagcgtggaagccatcgagggctctcatgtgtctctgctgtgtggcgccgacagcaatcctcctcctctgctgacctggatgagagatggcaccgtgctgagagaagccgtggccgaatctctgctgctggaactggaagaagtgacccctgccgaggatggcgtgtacgcttgtctggccgagaatgcctacggccaggacaatagaaccgtgggcctgtccgtgatgtacgccccttggaagcctaccgtgaacggcacaatggtggccgtggaaggcgagacagtgtccatcctgtgtagcacccagagcaaccccgatcctatcctgaccatcttcaaagagaagcagatcctgagcaccgtgatctacgagagcgaactgcagctcgaactgcccgctgtgtccccagaggatgatggcgaatattggtgcgtggcagagaaccagtacggccagagagccaccgccttcaacctgagcgtggaatttgctcccgtgctgctgctcgagagccattgtgctgccgccagagataccgtgcagtgcctgtgtgtggtcaagtctaaccccgagcctagcgtggcctttgagctgcccagcagaaacgtgaccgtgaatgagagcgagcgcgagttcgtgtacagcgagagatctggactggtgctgaccagcatcctgacactgagaggacaggctcaggcccctcctagagtgatctgcaccgccagaaatctgtacggcgccaagagcctggaactgccatttcagggcgcccacagactcatgtgggccaagattggacct

SEQ ID NO: 5 - protein, 23 aa, synthetic construct
VAISTSTVLLCGLSAVSLLACYL

SEQ ID NO: 6 - DNA, 69 bp, synthetic construct
  source 1..69: mol_type = other DNA
gtggccatcagcacaagcaccgtgctgctgtgtggactgtctgccgtttctctgctggcctgctacctg

SEQ ID NO: 7 - protein, 27 aa, synthetic construct
FWVLVVVGGVLACYSLLVTVAFIIFWV

SEQ ID NO: 8 - DNA, 81 bp, synthetic construct
  source 1..81: mol_type = other DNA
ttctgggtgctcgtggttgttggcggagtgctggcctgttactctctgctggtcaccgtggccttcatcatcttttgggtc

SEQ ID NO: 9 - protein, 21 aa, synthetic construct
VAAILGLGLVLGLLGPLAILL

SEQ ID NO: 10 - DNA, 63 bp, synthetic construct
  source 1..63: mol_type = other DNA
gtggccgccattcttggactgggacttgttctgggactgctgggacctctggccattctgctg

SEQ ID NO: 11 - DNA, 63 bp, synthetic construct
  source 1..63: mol_type = other DNA
atctacatctgggcccctctggctggaacatgcggagtgttgctgctgagcctggtcatcacc

SEQ ID NO: 12 - protein, 21 aa, synthetic construct
MGLAFLVLVALVWFLVEDWLS

SEQ ID NO: 13 - DNA, 63 bp, synthetic construct
  source 1..63: mol_type = other DNA
atgggcctcgcctttctggtgctggtggcccttgtgtggttcctggtggaagattggctgagc

SEQ ID NO: 14 - protein, 21 aa, synthetic construct
FLVIIVILSALFLGTLACFCV

SEQ ID NO: 15 - DNA, 63 bp, synthetic construct
  source 1..63: mol_type = other DNA
ttcctggtcatcatcgtgatcctgagcgccctgttcctgggcaccctggcctgtttttgcgtg

SEQ ID NO: 16 - protein, 39 aa, synthetic construct
KSRQTPPLASVEMEAMEALPVTWGTSSRDEDLENCSHHL

SEQ ID NO: 17 - DNA, 117 bp, synthetic construct
  source 1..117: mol_type = other DNA
aagtccagacagacacctcctctggccagcgtggaaatggaagccatggaagctctgcctgtgacctggggcaccagctccagagatgaggacctggaaaactgctcccaccacctg

SEQ ID NO: 18 - protein, 41 aa, synthetic construct
RSKRSRLLHSDYMNMTPRRPGPTRKHYQPYAPPRDFAAYRS

SEQ ID NO: 19 - skipped

SEQ ID NO: 20 - protein, 6 aa, synthetic construct
GGSGGS

SEQ ID NO: 21 - protein, 9 aa, synthetic construct
GGSGGSGGS

SEQ ID NO: 22 - protein, 12 aa, synthetic construct
GGSGGSGGSGGS

SEQ ID NO: 23 - protein, 15 aa, synthetic construct
GGSGGSGGSGGSGGS

SEQ ID NO: 24 - protein, 4 aa, synthetic construct
GGGS

SEQ ID NO: 25 - protein, 8 aa, synthetic construct
GGGSGGGS

SEQ ID NO: 26 - protein, 12 aa, synthetic construct
GGGSGGGSGGGS

SEQ ID NO: 27 - protein, 16 aa, synthetic construct
GGGSGGGSGGGSGGGS

SEQ ID NO: 28 - protein, 20 aa, synthetic construct
GGGSGGGSGGGSGGGSGGGS

SEQ ID NO: 29 - protein, 5 aa, synthetic construct
GGGGS

SEQ ID NO: 30 - protein, 10 aa, synthetic construct
GGGGSGGGGS

SEQ ID NO: 31 - protein, 15 aa, synthetic construct
GGGGSGGGGSGGGGS

SEQ ID NO: 32 - protein, 20 aa, synthetic construct
GGGGSGGGGSGGGGSGGGGS

SEQ ID NO: 33 - protein, 25 aa, synthetic construct
GGGGSGGGGSGGGGSGGGGSGGGGS

SEQ ID NO: 34 - protein, 18 aa, synthetic construct
GSTSGSGKPGSGEGSTKG

SEQ ID NO: 35 - protein, 20 aa, synthetic construct
EAAAKEAAAKEAAAKEAAAK

SEQ ID NO: 36 - protein, 21 aa, Homo sapiens
IYIWAPLAGTCGVLLLSLVIT

SEQ ID NO: 37 - protein, 27 aa, Homo sapiens
IYIWAPLAGTCGVLLLSLVITLYCNHR

SEQ ID NO: 38 - protein, 28 aa, Homo sapiens
IYIWAPLAGTCGVLLLSLVITLYCNHRN

SEQ ID NO: 39 - skipped

SEQ ID NO: 40 - protein, 42 aa, Homo sapiens
AAAIEVMYPPPYLDNEKSNGTIIHVKGKHLCPSPLFPGPSKP

SEQ ID NO: 41 - protein, 12 aa, Homo sapiens
ESKYGPPCPSCP

SEQ ID NO: 42 - protein, 12 aa, Homo sapiens
ESKYGPPAPSAP

SEQ ID NO: 43 - protein, 12 aa, Homo sapiens
ESKYGPPCPPCP

SEQ ID NO: 44 - protein, 12 aa, Homo sapiens
EPKSCDKTHTCP

SEQ ID NO: 45 - protein, 86 aa, Homo sapiens
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 46 - protein, 132 aa, Homo sapiens
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 47 - protein, 34 aa, Homo sapiens
ACPTGLYTHSGECCKACNLGEGVAQPCGANQTVC

SEQ ID NO: 48 - protein, 20 aa, Homo sapiens
AVGQDTQEVIVVPHSLPFKV

SEQ ID NO: 49 - protein, 45 aa, Homo sapiens
TTTPAPRPPTPAPTIALQPLSLRPEACRPAAGGAVHTRGLDFACD

SEQ ID NO: 50 - protein, 66 aa, Homo sapiens
ALSNSIMYFSHFVPVFLPAKPTTTPAPRPPTPAPTIASQPLSLRPEACRPAAGGAVHTRGLDFACD

SEQ ID NO: 51 - protein, 55 aa, Homo sapiens
FVPVFLPAKPTTTPAPRPPTPAPTIALQPLSLRPEACRPAAGGAVHTRGLDFACD

SEQ ID NO: 52 - DNA, 135 bp, Homo sapiens
  source 1..135: mol_type = other DNA
accacaactcctgctcctagacctcctacaccagctcctacaatcgccctgcagccactgtctctgaggccagaagcttgtagacctgctgcaggcggagccgtgcatacaagaggactggatttcgcctgcgac

SEQ ID NO: 53 - DNA, 126 bp, Homo sapiens
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 54 - DNA, 36 bp, Homo sapiens
  source 1..36: mol_type = other DNA
gaaagcaagtacggtccaccttgccctagctgtccg

SEQ ID NO: 55 - DNA, 36 bp, Homo sapiens
  source 1..36: mol_type = other DNA
gaatccaagtacggccccccagcgcctagtgcccca

SEQ ID NO: 56 - DNA, 36 bp, Homo sapiens
  source 1..36: mol_type = other DNA
gaatctaaatatggcccgccatgcccgccttgccca

SEQ ID NO: 57 - DNA, 36 bp, Homo sapiens
  source 1..36: mol_type = other DNA
gaaccgaagtcttgtgataaaactcatacgtgcccg

SEQ ID NO: 58 - DNA, 258 bp, Homo sapiens
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 59 - DNA, 396 bp, Homo sapiens
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 60 - DNA, 102 bp, Homo sapiens
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 61 - DNA, 60 bp, Homo sapiens
  source 1..60: mol_type = other DNA
gctgtgggccaggacacgcaggaggtcatcgtggtgccacactccttgccctttaaggtg

SEQ ID NO: 62 - protein, 12 aa, synthetic construct
GGCKJSGGCKJS

SEQ ID NO: 63 - DNA, 123 bp, synthetic construct
  source 1..123: mol_type = other DNA
cgaagcaagcggagccggctgctgcacagcgactacatgaacatgacccctagacggcccggacctaccagaaagcactaccagccttacgctcctcctagagatttcgccgcctaccggtcc

SEQ ID NO: 64 - protein, 42 aa, synthetic construct
ALYLLRRDQRLPPDAHKPPGGGSFRTPIQEEQADAHSTLAKI

SEQ ID NO: 65 - DNA, 126 bp, synthetic construct
  source 1..126: mol_type = other DNA
gctctgtacctgctgagaagggaccagagactgcctcctgacgctcacaaacctccaggcggaggcagcttcagaacccctatccaagaggaacaggctgacgcccacagcaccctggccaagatc

SEQ ID NO: 66 - protein, 42 aa, synthetic construct
KRGRKKLLYIFKQPFMRPVQTTQEEDGCSCRFPEEEEGGCEL

SEQ ID NO: 67 - protein, 25 aa, synthetic construct
RKRTRERASRASTWEGRRRLNTQTL

SEQ ID NO: 68 - DNA, 75 bp, synthetic construct
  source 1..75: mol_type = other DNA
cggaagcggacaagagagagagccagcagagcctctacctgggagggaagaagaaggctgaacacccagacactc

SEQ ID NO: 69 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 70 - protein, 18 aa, Homo sapiens
MDWTWILFLVAAATRVHS

SEQ ID NO: 71 - protein, 20 aa, Homo sapiens
MEAPAQLLFLLLLWLPDTTG

SEQ ID NO: 72 - protein, 23 aa, Homo sapiens
MNRGVPFRHLLLVLQLALLPAAT

SEQ ID NO: 73 - protein, 20 aa, Mus musculus
METDTLLLWVLLLWVPGSTG

SEQ ID NO: 74 - protein, 21 aa, Mus musculus
MVPCTLLLLLAAALAPTQTRA

SEQ ID NO: 75 - protein, 18 aa, Homo sapiens
MKWVTFISLLFLFSSAYS

SEQ ID NO: 76 - protein, 20 aa, Homo sapiens
MYRMQLLSCIALSLALVTNS

SEQ ID NO: 77 - protein, 24 aa, Homo sapiens
MAVMAPRTLLLLLSGALALTQTWA

SEQ ID NO: 78 - protein, 20 aa, Homo sapiens
MSRSVALAVLALLSLSGLEA

SEQ ID NO: 79 - protein, 344 aa, Pan troglodytes
MEKFTWHTRGITCPPPMSVEHADIWVKSYSLYSRERYICNSGFKRKAGTSSLTECVLNKATNVAHWTTPSLKCIRDPALVLQRPVPPSTVTTAGMTPQPESLSPSGKGSVAVTMEDTFFMEREQHSYATPLQCRGSHLPGKDKEDREDAEHKAASVPERKLIRTLSSHTARRVQARAQASRVSHAAAPSVLGQRSFTCSQLSLQQPAASSPSSNTTAATTAAIVPGSQLMPSKSPSTGTTEIGSHESSHGTPSQTTAKTWELTASASHQPPGVYPQGHSDTTVAISTSTVLLCGLSAVSLLACYLKSRQTPPLASVEMEAMEALPVTGGTSSRDEDLENCSHHL

SEQ ID NO: 80 - RNA, 1035 nt, Pan troglodytes
  source 1..1035: mol_type = other RNA
atggagaaattcacctggcacacaagaggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttctccaaaggccagtgccaccctccacagtaacgacggcagggatgaccccacagccagagagcctctccccttctggaaaaggttccgtggctgtcacaatggaagacacatttttcatggagagggaacagcacagttacgccacacccttacagtgcaggggcagccaccttccagggaaggacaaggaagacagggaagacgctgaacacaaggcagcctctgttcctgagcgcaagctcatcaggacgctttcctcccacacagcgcggagagttcaggccagagcccaggcttcccgtgtttcacacgcagccgctccgagcgtcctcggccagcgcagcttcacctgctctcagctgagcctccagcagcccgcagcttcatctcccagctcaaacaccacagcggccacaacagcagctattgtcccgggctcccagctgatgccttcaaaatcaccttccacaggaaccacagagataggcagtcatgagtcctcccacggcaccccctctcagacaacagccaagacctgggaactcacagcatctgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgactggggggaccagcagcagagatgaagacttggaaaactgctctcaccacctatga

SEQ ID NO: 81 - protein, 263 aa, Mus musculus
MASPQLRGYGVQAIPVLLLLLLLLLLPLRVTPGTTCPPPVSIEHADIRVKNYSVNSRERYVCNSGFKRKAGTSTLIECVINKNTNVAHWTTPSLKCIRDPSLAHYSPVPTVVTPKVTSQPESPSPSAKEPEAFSPKSDTAMTTETAIMPGSRLTPSQTTSAGTTGTGSHKSSRAPSLAATMTLEPTASTSLRITEISPHSSKMTKVAISTSVLLVGAGVVMAFLAWYIKSRQPSQPCRVEVETMETVPMTVRASSKEDEDTGA

SEQ ID NO: 82 - RNA, 1664 nt, Mus musculus
  source 1..1664: mol_type = other RNA
tctctgcaatccgggggctaaagcaaaagcgaaagcgaatactccttagcagcggtctggtcctggagctgagctcgccacctcccagggaccccagccttgcgtcccgttgggtcactgctggggacaattggccatggcctcgccgcagctccggggctatggagtccaggccattcctgtgttgctgctgctgctgttgctactgttgctcccgctgagggtgacgccgggcaccacgtgtccacctcccgtatctattgagcatgctgacatccgggtcaagaattacagtgtgaactccagggagaggtatgtctgtaactctggctttaagcggaaagctggaacatccaccctgattgagtgtgtgatcaacaagaacacaaatgttgcccactggacaactcccagcctcaagtgcatcagagacccctccctagctcactacagtccagtgccaacagtagtgacaccaaaggtgacctcacagccagagagcccctccccctctgcaaaagagccagaagctttctctcccaaatcagataccgcaatgaccacagagacagctattatgcctggctccaggctgacaccatcccaaacaacttctgcaggaactacagggacaggcagtcacaagtcctcccgagccccatctcttgcagcaacaatgaccttggagcctacagcctccacctccctcaggataacagagatttctccccacagttccaaaatgacgaaagtggccatctctacatcggtcctcttggttggtgcaggggttgtgatggctttcctggcctggtacatcaaatcaaggcagccttctcagccgtgccgtgttgaggtggaaaccatggaaacagtaccaatgactgtgagggccagcagcaaggaggatgaagacacaggagcctaaccacacgatttcagaaactcagggagacctgcccagctgatcctgaagagaagatacaagggcagttagtccagctgcgcctggacacaggagaaagcccaatgtgataatagaggtctctggtatgatctgttttactgagcctgggtgcctcccatattcaggcctggatccccggagtccggaaggacaccacctttcttcctggttcatcacagaggccaacttcccagagtacaagcagcctgagctctcctgggagtctccattaaaagcctcgagttccactcagagattaactcaaagcagctgatctgctagactctttttctattccctactctgacttgcagtttacagagattgacaaggctcccattgtcttccaaggctcctctggcacaggagatgtctgtaaagaagacagcaacatttgagctcctgaagacttggccctttgctgctttgcacctattggaggagagcagagaacagaagaagagatactgagccaatgaaccctttcgtataggattcatgacaaaaccaaactcagtgactatatatgtatgtgtgtgtgtgtgtgtatgtaaaagtgtatatttacatatacatttatatttatactttcttttctattatatctacatattgtatatgatttatatttgaaagtgctttgtgtagacaaaataaaatatctattttcagtacaaaaagcattaaaattatcaac

SEQ ID NO: 83 - RNA, 765 nt, Rattus norvegicus
  source 1..765: mol_type = other RNA
atggagcatcacagagaatcgtggaaattatggggacccaacagggaagacatcgagaagggcatcacgtgcccaacgcccatatctattgaacacgcagacatccgggtcaagaattacagtgtgaactccagggagaggtatgtctgtaactctggcttcaagcggaaagcaggcacatccaccctgaccgagtgcgtgatcaataagaacacgaatgtggcccactggacaactcccaacctcaagtgtatcaagccagaagctttatctcccaaatcagataccacagtggccacagagacagctattgtgcctggctccaggctgacaccatcccaagcagcttctgcaggaactacagggacgggcagacacaagtcctccccagccccatctcttgcaacaacaatgaccttggagcctacagcctccacctccctcaggataacagagatttctccgcacagttccgaaatgacaaaaggtgagtattttcttttgtttgatgtgtttataaccagggcactacaaagcagacccagcatgctcatcagcctggtccaggccactctagcacagtcgccagcagtatgccctttgcctcgccgaaatgtctccgttagagagcaagcttctcagccgcgtcgtgttgaggtggaaaccatggaaacagtaccaatgactgtgagggcccgcagcaaggaggacgaagaccacacgacttcagaaactcagggagaccagcccacgcgcggagtctga

SEQ ID NO: 84 - protein, 254 aa, Rattus norvegicus
MEHHRESWKLWGPNREDIEKGITCPTPISIEHADIRVKNYSVNSRERYVCNSGFKRKAGTSTLTECVINKNTNVAHWTTPNLKCIKPEALSPKSDTTVATETAIVPGSRLTPSQAASAGTTGTGRHKSSPAPSLATTMTLEPTASTSLRITEISPHSSEMTKGEYFLLFDVFITRALQSRPSMLISLVQATLAQSPAVCPLPRRNVSVREQASQPRRVEVETMETVPMTVRARSKEDEDHTTSETQGDQPTRGV

SEQ ID NO: 85 - protein, 199 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIR

SEQ ID NO: 86 - protein, 389 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 87 - skipped

SEQ ID NO: 88 - protein, 391 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLRSKRSRLLHSDYMNMTPRRPGPTRKHYQPYAPPRDFAAYRS

SEQ ID NO: 89 - protein, 217 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIR

SEQ ID NO: 90 - protein, 330 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 91 - protein, 327 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 92 - protein, 330 aa, synthetic construct; identical to SEQ ID NO: 90

SEQ ID NO: 93 - protein, 286 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWV

SEQ ID NO: 94 - protein, 325 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVKSRQTPPLASVEMEAMEALPVTWGTSSRDEDLENCSHHL

SEQ ID NO: 95 - protein, 564 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPFWVLVVVGGVLACYSLLVTVAFIIFWVRSKRSRLLHSDYMNMTPRRPGPTRKHYQPYAPPRDFAAYRS

SEQ ID NO: 96 - protein, 325 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRTTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKI

SEQ ID NO: 97 - protein, 325 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRTTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCEL

SEQ ID NO: 98 - protein, 308 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRTTTPAPRPPTPAPTIALQPLSLRPEACRPAAGGAVHTRGLDFACDMGLAFLVLVALVWFLVEDWLSRKRTRERASRASTWEGRRRLNTQTL

SEQ ID NO: 99 - protein, 403 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRTTTPAPRPPTPAPTIALQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 100 - protein, 328 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVALYLLRRDQRLPPDAHKPPGGGSFRTPIQEEQADAHSTLAKI

SEQ ID NO: 101 - protein, 328 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 102 - protein, 311 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVRKRTRERASRASTWEGRRRLNTQTL

SEQ ID NO: 103 - protein, 406 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 104 - protein, 110 aa, synthetic construct
DPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 105 - DNA, 360 bp, synthetic construct
  source 1..360: mol_type = other DNA
tggcgccgcaagcggaaagagaagcagtctgagacaagccccaaagagttcctgaccatctacgaggacgtgaaggacctgaaaacccggcggaaccacgagcaagagcagacctttcctggcggcggaagcaccatctacagcatgatccagagccagtctagcgcccctaccagccaagagcctgcctacacactgtactccctgatccagcctagcagaaagagcggcagccggaagagaaatcacagccccagcttcaacagcacgatctacgaagtgatcggcaagagccagccaaaggctcagaaccctgccaggctgagccggaaagagctggaaaacttcgacgtgtacagc

SEQ ID NO: 106 - DNA, 1167 bp, synthetic construct
  source 1..1167: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagatcccgctctggtgcatcagcgtcctgctcctccaagcacagtgacaacagctggcgtgacaccccagcctgagtctctgagcccttctggaaaagagcctgccgccagctctcccagctctaacaatactgccgccaccacagccgccatcgtgcctggatctcaactgatgcctagcaagagccctagcaccggcaccacagagatcagctctcacgagtctagccacggcaccccttctcagaccaccgccaagaattgggagctgacagccagcgcctctcatcaaccacctggcgtgtaccctcagggccacagcgatacaacagtggccatcagcacaagcaccgtgctgctgtgtggactgtctgccgtttctctgctggcctgctacctgaagtccagacagacacctcctctggccagcgtggaaatggaagccatggaagctctgcctgtgacctggggcaccagctccagagatgaggacctggaaaactgctcccaccacctg

SEQ ID NO: 107 - DNA, 1173 bp, synthetic construct
  source 1..1173: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagatcccgctctggtgcatcagcgtcctgctcctccaagcacagtgacaacagctggcgtgacaccccagcctgagtctctgagcccttctggaaaagagcctgccgccagctctcccagctctaacaatactgccgccaccacagccgccatcgtgcctggatctcaactgatgcctagcaagagccctagcaccggcaccacagagatcagctctcacgagtctagccacggcaccccttctcagaccaccgccaagaattgggagctgacagccagcgcctctcatcaaccacctggcgtgtaccctcagggccacagcgatacaacagtggccatcagcacaagcaccgtgctgctgtgtggactgtctgccgtttctctgctggcctgctacctgcgaagcaagcggagccggctgctgcacagcgactacatgaacatgacccctagacggcccggacctaccagaaagcactaccagccttacgctcctcctagagatttcgccgcctaccggtcc

SEQ ID NO: 108 - DNA, 1311 bp, synthetic construct
  source 1..1311: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagatcccgctctggtgcatcagcgtcctgctcctccaagcacagtgacaacagctggcgtgacaccccagcctgagtctctgagcccttctggaaaagagcctgccgccagctctcccagctctaacaatactgccgccaccacagccgccatcgtgcctggatctcaactgatgcctagcaagagccctagcaccggcaccacagagatcagctctcacgagtctagccacggcaccccttctcagaccaccgccaagaattgggagctgacagccagcgcctctcatcaaccacctggcgtgtaccctcagggccacagcgatacaacagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagatttcgccgcctaccggtcc

SEQ ID NO: 109 - DNA, 1320 bp, synthetic construct
  source 1..1320: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagatcccgctctggtgcatcagcgtcctgctcctccaagcacagtgacaacagctggcgtgacaccccagcctgagtctctgagcccttctggaaaagagcctgccgccagctctcccagctctaacaatactgccgccaccacagccgccatcgtgcctggatctcaactgatgcctagcaagagccctagcaccggcaccacagagatcagctctcacgagtctagccacggcaccccttctcagaccaccgccaagaattgggagctgacagccagcgcctctcatcaaccacctggcgtgtaccctcagggccacagcgatacaacaaccacaactcctgctcctagacctcctacaccagctcctacaatcgccctgcagccactgtctctgaggccagaagcttgtagacctgctgcaggcggagccgtgcatacaagaggactggatttcgcctgcgacttctgggtgctcgtggttgttggcggagtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagatttcgccgcctaccggtcc

SEQ ID NO: 110 - DNA, 981 bp, synthetic construct
  source 1..981: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagatttcgccgcctaccggtcc

SEQ ID NO: 111 - DNA, 990 bp, synthetic construct
  source 1..990: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagaaccacaactcctgctcctagacctcctacaccagctcctacaatcgccctgcagccactgtctctgaggccagaagcttgtagacctgctgcaggcggagccgtgcatacaagaggactggatttcgcctgcgacttctgggtgctcgtggttgttggcggagtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagatttcgccgcctaccggtcc

SEQ ID NO: 112 - DNA, 858 bp, synthetic construct
  source 1..858: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtc

SEQ ID NO: 113 - DNA, 975 bp, synthetic construct
  source 1..975: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtcaagtccagacagacacctcctctggccagcgtggaaatggaagccatggaagctctgcctgtgacctggggcaccagctccagagatgaggacctggaaaactgctcccaccacctg

SEQ ID NO: 114 - DNA, 1692 bp, synthetic construct
  source 1..1692: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagataccctccagtgatcgtggaaatgaacagcagcgtggaagccatcgagggctctcatgtgtctctgctgtgtggcgccgacagcaatcctcctcctctgctgacctggatgagagatggcaccgtgctgagagaagccgtggccgaatctctgctgctggaactggaagaagtgacccctgccgaggatggcgtgtacgcttgtctggccgagaatgcctacggccaggacaatagaaccgtgggcctgtccgtgatgtacgccccttggaagcctaccgtgaacggcacaatggtggccgtggaaggcgagacagtgtccatcctgtgtagcacccagagcaaccccgatcctatcctgaccatcttcaaagagaagcagatcctgagcaccgtgatctacgagagcgaactgcagctcgaactgcccgctgtgtccccagaggatgatggcgaatattggtgcgtggcagagaaccagtacggccagagagccaccgccttcaacctgagcgtggaatttgctcccgtgctgctgctcgagagccattgtgctgccgccagagataccgtgcagtgcctgtgtgtggtcaagtctaaccccgagcctagcgtggcctttgagctgcccagcagaaacgtgaccgtgaatgagagcgagcgcgagttcgtgtacagcgagagatctggactggtgctgaccagcatcctgacactgagaggacaggctcaggcccctcctagagtgatctgcaccgccagaaatctgtacggcgccaagagcctggaactgccatttcagggcgcccacagactcatgtgggccaagattggacctttctgggtgctcgtggttgttggcggagtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagatttcgccgcctaccggtcc

SEQ ID NO: 115 - DNA, 975 bp, synthetic construct
  source 1..975: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagaaccacaactcctgctcctagacctcctacaccagctcctacaatcgccctgcagccactgtctctgaggccagaagcttgtagacctgctgcaggcggagccgtgcatacaagaggactggatttcgcctgcgacgtggccgccattcttggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatc

SEQ ID NO: 116 - DNA, 975 bp, synthetic construct
  source 1..975: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagaaccacaactcctgctcctagacctcctacaccagctcctacaatcgccctgcagccactgtctctgaggccagaagcttgtagacctgctgcaggcggagccgtgcatacaagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 117 - DNA, 924 bp, synthetic construct
  source 1..924: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagaaccacaactcctgctcctagacctcctacaccagctcctacaatcgccctgcagccactgtctctgaggccagaagcttgtagacctgctgcaggcggagccgtgcatacaagaggactggatttcgcctgcgacatgggcctcgcctttctggtgctggtggcccttgtgtggttcctggtggaagattggctgagccggaagcggacaagagagagagccagcagagcctctacctgggagggaagaagaaggctgaacacccagacactc

SEQ ID NO: 118 - DNA, 1209 bp, synthetic construct
  source 1..1209: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagaaccacaactcctgctcctagacctcctacaccagctcctacaatcgccctgcagccactgtctctgaggccagaagcttgtagacctgctgcaggcggagccgtgcatacaagaggactggatttcgcctgcgacttcctggtcatcatcgtgatcctgagcgccctgttcctgggcaccctggcctgtttttgcgtgtggcgccgcaagcggaaagagaagcagtctgagacaagccccaaagagttcctgaccatctacgaggacgtgaaggacctgaaaacccggcggaaccacgagcaagagcagacctttcctggcggcggaagcaccatctacagcatgatccagagccagtctagcgcccctaccagccaagagcctgcctacacactgtactccctgatccagcctagcagaaagagcggcagccggaagagaaatcacagccccagcttcaacagcacgatctacgaagtgatcggcaagagccagccaaaggctcagaaccctgccaggctgagccggaaagagctggaaaacttcgacgtgtacagc

SEQ ID NO: 119 - DNA, 984 bp, synthetic construct
  source 1..984: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtcgctctgtacctgctgagaagggaccagagactgcctcctgacgctcacaaacctccaggcggaggcagcttcagaacccctatccaagaggaacaggctgacgcccacagcaccctggccaagatc

SEQ ID NO: 120 - DNA, 984 bp, synthetic construct
  source 1..984: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 121 - DNA, 933 bp, synthetic construct
  source 1..933: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtccggaagcggacaagagagagagccagcagagcctctacctgggagggaagaagaaggctgaacacccagacactc

SEQ ID NO: 122 - DNA, 1218 bp, synthetic construct
  source 1..1218: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtctggcgccgcaagcggaaagagaagcagtctgagacaagccccaaagagttcctgaccatctacgaggacgtgaaggacctgaaaacccggcggaaccacgagcaagagcagacctttcctggcggcggaagcaccatctacagcatgatccagagccagtctagcgcccctaccagccaagagcctgcctacacactgtactccctgatccagcctagcagaaagagcggcagccggaagagaaatcacagccccagcttcaacagcacgatctacgaagtgatcggcaagagccagccaaaggctcagaaccctgccaggctgagccggaaagagctggaaaacttcgacgtgtacagc

SEQ ID NO: 123 - protein, 21 aa, Homo sapiens
MALPVTALLLPLALLLHAARP

SEQ ID NO: 124 - protein, 5 aa, synthetic construct
  REGION 1..5: note = The entire sequence of amino acids 1-5 can be repeated one or more times.
GGGGS

SEQ ID NO: 125 - protein, 4 aa, synthetic construct
  REGION 1..4: note = The entire sequence of amino acids 1-4 can be repeated one or more times.
GGGS

SEQ ID NO: 126 - protein, 15 aa, synthetic construct
EAAAKEAAAKEAAAK

SEQ ID NO: 127 - protein, 20 aa, synthetic construct
SGGSGGGGSGGGSGGGGSLQ

SEQ ID NO: 128 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctg